SEQUENCE LISTING (68 sequences)

SEQ ID NO: 1 - DNA, 20 bp, synthetic construct
  misc_feature 1..20: note = Single strand DNA oligonucleotide
  source 1..20: mol_type = other DNA
agacaccgctacacatctgc

SEQ ID NO: 2 - DNA, 20 bp, synthetic construct
  misc_feature 1..20: note = Single strand DNA oligonucleotide
  source 1..20: mol_type = other DNA
gggaagtttgtgccccagta

SEQ ID NO: 3 - DNA, 20 bp, synthetic construct
  misc_feature 1..20: note = Single strand DNA oligonucleotide
  source 1..20: mol_type = other DNA
gcattctgagaggatgcggt

SEQ ID NO: 4 - DNA, 20 bp, synthetic construct
  misc_feature 1..20: note = Single strand DNA oligonucleotide
  source 1..20: mol_type = other DNA
tggccttggccttcctattc

SEQ ID NO: 5 - protein, 404 aa, synthetic construct
  REGION 1..404: note = Fc-gamma receptor 1 (CD64) AA SEQUENCE
MILTSFGDDMWLLTTLLLWVPVGGEVVNATKAVITLQPPWVSIFQKENVTLWCEGPHLPGDSSTQWFINGTAVQISTPSYSIPEASFQDSGEYRCQIGSSMPSDPVQLQIHNDWLLLQASRRVLTEGEPLALRCHGWKNKLVYNVVFYRNGKSFQFSSDSEVAILKTNLSHSGIYHCSGTGRHRYTSAGVSITVKELFTTPVLRASVSSPFPEGSLVTLNCETNLLLQRPGLQLHFSFYVGSKILEYRNTSSEYHIARAEREDAGFYWCEVATEDSSVLKRSPELELQVLGPQSSAPVWFHILFYLSVGIMFSLNTVLYVKIHRLQREKKYNLEVPLVSEQGKKANSFQQVRSDGVYEEVTATASQTTPKEAPDGPRSSVGDCGPEQPEPLPPSDSTGAQTSQS

SEQ ID NO: 6 - DNA, 1215 bp, synthetic construct
  misc_feature 1..1215: note = Fc-gamma receptor 1 (CD64) NA SEQUENCE
  source 1..1215: mol_type = other DNA
atgattcttaccagctttggagatgacatgtggcttctaacaactctgctactttgggttccagtcggtggggaagtggttaatgccaccaaggctgtgatcaccttgcagcctccatgggtcagtattttccagaaggaaaatgtcactttatggtgtgaggggcctcacctgcctggagacagttccacacaatggtttatcaacggaacagccgttcagatctccacgcctagttatagcatcccagaggccagttttcaggacagtggcgaatacaggtgtcagataggttcctcaatgccaagtgaccctgtgcagttgcaaatccacaatgattggctgctactccaggcctcccgcagagtcctcacagaaggagaacccctggccttgaggtgtcacggatggaagaataaactggtgtacaatgtggttttctatagaaatggaaaatcctttcagttttcttcagattcggaggtcgccattctgaaaaccaacctgagtcacagcggcatctaccactgctcaggcacgggaagacaccgctacacatctgcaggagtgtccatcacggtgaaagagctgtttaccacgccagtgctgagagcatccgtgtcatctcccttcccggaggggagtctggtcaccctgaactgtgagacgaatttgctcctgcagagacccggcttacagcttcacttctccttctacgtgggcagcaagatcctggagtacaggaacacatcctcagagtaccatatagcaagggcggaaagagaagatgctggattctactggtgtgaggtagccacggaggacagcagtgtccttaagcgcagccctgagttggagctccaagtgcttggtccccagtcatcagctcctgtctggtttcacatcctgttttatctgtcagtgggaataatgttttcgttgaacacggttctctatgtgaaaatacacaggctgcagagagagaagaaatacaacttagaagtccctttggtttctgagcagggaaagaaagcaaattcctttcagcaagttagaagcgatggcgtgtatgaagaagtaacagccactgcgagccagaccacaccaaaagaagcgcccgatggacctcgaagctcagtgggtgactgtggacccgagcagcctgaaccccttcctcccagtgacagtactggggcacaaacttcccaaagttga

SEQ ID NO: 7 - protein, 273 aa, synthetic construct
  REGION 1..273: note = Extracellular domain of CD64 AA sequence
EVVNATKAVITLQPPWVSIFQKENVTLWCEGPHLPGDSSTQWFINGTAVQISTPSYSIPEASFQDSGEYRCQIGSSMPSDPVQLQIHNDWLLLQASRRVLTEGEPLALRCHGWKNKLVYNVVFYRNGKSFQFSSDSEVAILKTNLSHSGIYHCSGTGRHRYTSAGVSITVKELFTTPVLRASVSSPFPEGSLVTLNCETNLLLQRPGLQLHFSFYVGSKILEYRNTSSEYHIARAEREDAGFYWCEVATEDSSVLKRSPELELQVLGPQSSAP

SEQ ID NO: 8 - DNA, 819 bp, synthetic construct
  misc_feature 1..819: note = Extracellular domain of CD64 NA sequence
  source 1..819: mol_type = other DNA
gaagtggttaatgccaccaaggctgtgatcaccttgcagcctccatgggtcagtattttccagaaggaaaatgtcactttatggtgtgaggggcctcacctgcctggagacagttccacacaatggtttatcaacggaacagccgttcagatctccacgcctagttatagcatcccagaggccagttttcaggacagtggcgaatacaggtgtcagataggttcctcaatgccaagtgaccctgtgcagttgcaaatccacaatgattggctgctactccaggcctcccgcagagtcctcacagaaggagaacccctggccttgaggtgtcacggatggaagaataaactggtgtacaatgtggttttctatagaaatggaaaatcctttcagttttcttcagattcggaggtcgccattctgaaaaccaacctgagtcacagcggcatctaccactgctcaggcacgggaagacaccgctacacatctgcaggagtgtccatcacggtgaaagagctgtttaccacgccagtgctgagagcatccgtgtcatctcccttcccggaggggagtctggtcaccctgaactgtgagacgaatttgctcctgcagagacccggcttacagcttcacttctccttctacgtgggcagcaagatcctggagtacaggaacacatcctcagagtaccatatagcaagggcggaaagagaagatgctggattctactggtgtgaggtagccacggaggacagcagtgtccttaagcgcagccctgagttggagctccaagtgcttggtccccagtcatcagctcct

SEQ ID NO: 9 - protein, 23 aa, synthetic construct
  REGION 1..23: note = Transmembrane domain of CD64 AA sequence
VWFHILFYLSVGIMFSLNTVLYV

SEQ ID NO: 10 - DNA, 69 bp, synthetic construct
  misc_feature 1..69: note = Transmembrane domain of CD64 NA sequence
  source 1..69: mol_type = other DNA
gtctggtttcacatcctgttttatctgtcagtgggaataatgttttcgttgaacacggttctctatgtg

SEQ ID NO: 11 - protein, 84 aa, synthetic construct
  REGION 1..84: note = Intracellular domain of CD64 AA sequence
KIHRLQREKKYNLEVPLVSEQGKKANSFQQVRSDGVYEEVTATASQTTPKEAPDGPRSSVGDCGPEQPEPLPPSDSTGAQTSQS

SEQ ID NO: 12 - DNA, 255 bp, synthetic construct
  misc_feature 1..255: note = Intracellular domain of CD64 NA sequence
  source 1..255: mol_type = other DNA
aaaatacacaggctgcagagagagaagaaatacaacttagaagtccctttggtttctgagcagggaaagaaagcaaattcctttcagcaagttagaagcgatggcgtgtatgaagaagtaacagccactgcgagccagaccacaccaaaagaagcgcccgatggacctcgaagctcagtgggtgactgtggacccgagcagcctgaaccccttcctcccagtgacagtactggggcacaaacttcccaaagttga

SEQ ID NO: 13 - protein, 86 aa, synthetic construct
  REGION 1..86: note = Fc receptor common Gamma chainAA SEQUENCE
MISAVILFLLLLVEQAAALGEPQLCYILDAVLFLYGIVLTLLYCRLKIQVRKAAIASREKADAVYTGLNTRSQETYETLKHEKPPQ

SEQ ID NO: 14 - DNA, 261 bp, synthetic construct
  misc_feature 1..261: note = Fc receptor common Gamma chainNA SEQUENCE
  source 1..261: mol_type = other DNA
atgatctcagccgtgatcttgttcttgctccttttggtggaacaagcagccgccctgggagagccgcagctctgctatatcctggatgctgtcctgtttttgtatggtattgtccttaccctactctactgtcgactcaagatccaggtccgaaaggcagctatagccagccgtgagaaagcagatgctgtctacacgggcctgaacacccggagccaggagacatatgagactctgaagcatgagaaaccaccccagtag

SEQ ID NO: 15 - protein, 86 aa, synthetic construct; identical to SEQ ID NO: 13
  REGION 1..86: note = Fc receptor common Gamma chainAA SEQUENCE (in construct)

SEQ ID NO: 16 - DNA, 261 bp, synthetic construct; identical to SEQ ID NO: 14
  misc_feature 1..261: note = Fc receptor common Gamma chainNA SEQUENCE (in construct)
  source 1..261: mol_type = other DNA

SEQ ID NO: 17 - protein, 164 aa, synthetic construct
  REGION 1..164: note = CD3 zeta AA SEQUENCE
MKWKVSVLACILHVRFPGAEAQSFGLLDPKLCYLLDGILFIYGVIITALYLRAKFSRSAETAANLQDPNQLYNELNLGRREEYDVLEKKRARDPEMGGKQQRRRNPQEGVYNALQKDKMAEAYSEIGTKGERRRGKGHDGLYQGLSTATKDTYDALHMQTLAPR

SEQ ID NO: 18 - DNA, 495 bp, synthetic construct
  misc_feature 1..495: note = CD3zeta NA sequence
  source 1..495: mol_type = other DNA
atgaagtggaaagtgtctgttctcgcctgcatcctccacgtgcggttcccaggagcagaggcacagagctttggtctgctggatcccaaactctgctacttgctagatggaatcctcttcatctacggagtcatcatcacagccctgtacctgagagcaaaattcagcaggagtgcagagactgctgccaacctgcaggaccccaaccagctctacaatgagctcaatctagggcgaagagaggaatatgacgtcttggagaagaagcgggctcgggatccagagatgggaggcaaacagcagaggaggaggaacccccaggaaggcgtatacaatgcactgcagaaagacaagatggcagaagcctacagtgagatcggcacaaaaggcgagaggcggagaggcaaggggcacgatggcctttaccagggtctcagcactgccaccaaggacacctatgatgccctgcatatgcagaccctggcccctcgctaa

SEQ ID NO: 19 - protein, 113 aa, synthetic construct
  REGION 1..113: note = CD3 zeta intracellular ITAMS domain AA sequence
RAKFSRSAETAANLQDPNQLYNELNLGRREEYDVLEKKRARDPEMGGKQQRRRNPQEGVYNALQKDKMAEAYSEIGTKGERRRGKGHDGLYQGLSTATKDTYDALHMQTLAPR

SEQ ID NO: 20 - DNA, 339 bp, synthetic construct
  misc_feature 1..339: note = CD3 zeta intracellular ITAMS domain NA sequence
  source 1..339: mol_type = other DNA
agagcaaaattcagcaggagtgcagagactgctgccaacctgcaggaccccaaccagctctacaatgagctcaatctagggcgaagagaggaatatgacgtcttggagaagaagcgggctcgggatccagagatgggaggcaaacagcagaggaggaggaacccccaggaaggcgtatacaatgcactgcagaaagacaagatggcagaagcctacagtgagatcggcacaaaaggcgagaggcggagaggcaaggggcacgatggcctttaccagggtctcagcactgccaccaaggacacctatgatgccctgcatatgcagaccctggcccctcgc

SEQ ID NO: 21 - protein, 508 aa, synthetic construct
  REGION 1..508: note = FcgRI-T2A-gamma CONSTRUCT AA SEQUENCE
MILTSFGDDMWLLTTLLLWVPVGGEVVNATKAVITLQPPWVSIFQKENVTLWCEGPHLPGDSSTQWFINGTAVQISTPSYSIPEASFQDSGEYRCQIGSSMPSDPVQLQIHNDWLLLQASRRVLTEGEPLALRCHGWKNKLVYNVVFYRNGKSFQFSSDSEVAILKTNLSHSGIYHCSGTGRHRYTSAGVSITVKELFTTPVLRASVSSPFPEGSLVTLNCETNLLLQRPGLQLHFSFYVGSKILEYRNTSSEYHIARAEREDAGFYWCEVATEDSSVLKRSPELELQVLGPQSSAPVWFHILFYLSVGIMFSLNTVLYVKIHRLQREKKYNLEVPLVSEQGKKANSFQQVRSDGVYEEVTATASQTTPKEAPDGPRSSVGDCGPEQPEPLPPSDSTGAQTSQSEGRGSLLTCGDVEENPGPMISAVILFLLLLVEQAAALGEPQLCYILDAVLFLYGIVLTLLYCRLKIQVRKAAIASREKADAVYTGLNTRSQETYETLKHEKPPQ

SEQ ID NO: 22 - DNA, 1527 bp, synthetic construct
  misc_feature 1..1527: note = FcgRI-T2A-gamma CONSTRUCT NA SEQUENCE
  source 1..1527: mol_type = other DNA
atgattcttaccagctttggagatgacatgtggcttctaacaactctgctactttgggttccagtcggtggggaagtggttaatgccaccaaggctgtgatcaccttgcagcctccatgggtcagtattttccagaaggaaaatgtcactttatggtgtgaggggcctcacctgcctggagacagttccacacaatggtttatcaacggaacagccgttcagatctccacgcctagttatagcatcccagaggccagttttcaggacagtggcgaatacaggtgtcagataggttcctcaatgccaagtgaccctgtgcagttgcaaatccacaatgattggctgctactccaggcctcccgcagagtcctcacagaaggagaacccctggccttgaggtgtcacggatggaagaataaactggtgtacaatgtggttttctatagaaatggaaaatcctttcagttttcttcagattcggaggtcgccattctgaaaaccaacctgagtcacagcggcatctaccactgctcaggcacgggaagacaccgctacacatctgcaggagtgtccatcacggtgaaagagctgtttaccacgccagtgctgagagcatccgtgtcatctcccttcccggaggggagtctggtcaccctgaactgtgagacgaatttgctcctgcagagacccggcttacagcttcacttctccttctacgtgggcagcaagatcctggagtacaggaacacatcctcagagtaccatatagcaagggcggaaagagaagatgctggattctactggtgtgaggtagccacggaggacagcagtgtccttaagcgcagccctgagttggagctccaagtgcttggtccccagtcatcagctcctgtctggtttcacatcctgttttatctgtcagtgggaataatgttttcgttgaacacggttctctatgtgaaaatacacaggctgcagagagagaagaaatacaacttagaagtccctttggtttctgagcagggaaagaaagcaaattcctttcagcaagttagaagcgatggcgtgtatgaagaagtaacagccactgcgagccagaccacaccaaaagaagcgcccgatggacctcgaagctcagtgggtgactgtggacccgagcagcctgaaccccttcctcccagtgacagtactggggcacaaacttcccaaagtgagggcagaggaagtctgctaacatgcggtgacgtcgaggagaatcctggcccaatgatctcagccgtgatcttgttcttgctccttttggtggaacaagcagccgccctgggagagccgcagctctgctatatcctggatgctgtcctgtttttgtatggtattgtccttaccctactctactgtcgactcaagatccaggtccgaaaggcagctatagccagccgtgagaaagcagatgctgtctacacgggcctgaacacccggagccaggagacatatgagactctgaagcatgagaaaccaccccagtag

SEQ ID NO: 23 - protein, 621 aa, synthetic construct
  REGION 1..621: note = FcgRI-T2A-FcRg-CD3zeta CONSTRUCT AA SEQUENCE
MILTSFGDDMWLLTTLLLWVPVGGEVVNATKAVITLQPPWVSIFQKENVTLWCEGPHLPGDSSTQWFINGTAVQISTPSYSIPEASFQDSGEYRCQIGSSMPSDPVQLQIHNDWLLLQASRRVLTEGEPLALRCHGWKNKLVYNVVFYRNGKSFQFSSDSEVAILKTNLSHSGIYHCSGTGRHRYTSAGVSITVKELFTTPVLRASVSSPFPEGSLVTLNCETNLLLQRPGLQLHFSFYVGSKILEYRNTSSEYHIARAEREDAGFYWCEVATEDSSVLKRSPELELQVLGPQSSAPVWFHILFYLSVGIMFSLNTVLYVKIHRLQREKKYNLEVPLVSEQGKKANSFQQVRSDGVYEEVTATASQTTPKEAPDGPRSSVGDCGPEQPEPLPPSDSTGAQTSQSEGRGSLLTCGDVEENPGPMISAVILFLLLLVEQAAALGEPQLCYILDAVLFLYGIVLTLLYCRLKIQVRKAAIASREKADAVYTGLNTRSQETYETLKHEKPPQRAKFSRSAETAANLQDPNQLYNELNLGRREEYDVLEKKRARDPEMGGKQQRRRNPQEGVYNALQKDKMAEAYSEIGTKGERRRGKGHDGLYQGLSTATKDTYDALHMQTLAPR

SEQ ID NO: 24 - DNA, 1866 bp, synthetic construct
  misc_feature 1..1866: note = FcgRI-T2A-FcRg-CD3zeta CONSTRUCT NA SEQUENCE
  source 1..1866: mol_type = other DNA
atgattcttaccagctttggagatgacatgtggcttctaacaactctgctactttgggttccagtcggtggggaagtggttaatgccaccaaggctgtgatcaccttgcagcctccatgggtcagtattttccagaaggaaaatgtcactttatggtgtgaggggcctcacctgcctggagacagttccacacaatggtttatcaacggaacagccgttcagatctccacgcctagttatagcatcccagaggccagttttcaggacagtggcgaatacaggtgtcagataggttcctcaatgccaagtgaccctgtgcagttgcaaatccacaatgattggctgctactccaggcctcccgcagagtcctcacagaaggagaacccctggccttgaggtgtcacggatggaagaataaactggtgtacaatgtggttttctatagaaatggaaaatcctttcagttttcttcagattcggaggtcgccattctgaaaaccaacctgagtcacagcggcatctaccactgctcaggcacgggaagacaccgctacacatctgcaggagtgtccatcacggtgaaagagctgtttaccacgccagtgctgagagcatccgtgtcatctcccttcccggaggggagtctggtcaccctgaactgtgagacgaatttgctcctgcagagacccggcttacagcttcacttctccttctacgtgggcagcaagatcctggagtacaggaacacatcctcagagtaccatatagcaagggcggaaagagaagatgctggattctactggtgtgaggtagccacggaggacagcagtgtccttaagcgcagccctgagttggagctccaagtgcttggtccccagtcatcagctcctgtctggtttcacatcctgttttatctgtcagtgggaataatgttttcgttgaacacggttctctatgtgaaaatacacaggctgcagagagagaagaaatacaacttagaagtccctttggtttctgagcagggaaagaaagcaaattcctttcagcaagttagaagcgatggcgtgtatgaagaagtaacagccactgcgagccagaccacaccaaaagaagcgcccgatggacctcgaagctcagtgggtgactgtggacccgagcagcctgaaccccttcctcccagtgacagtactggggcacaaacttcccaaagtgagggcagaggaagtctgctaacatgcggtgacgtcgaggagaatcctggcccaatgatctcagccgtgatcttgttcttgctccttttggtggaacaagcagccgccctgggagagccgcagctctgctatatcctggatgctgtcctgtttttgtatggtattgtccttaccctactctactgtcgactcaagatccaggtccgaaaggcagctatagccagccgtgagaaagcagatgctgtctacacgggcctgaacacccggagccaggagacatatgagactctgaagcatgagaaaccaccccagagagcaaaattcagcaggagtgcagagactgctgccaacctgcaggaccccaaccagctctacaatgagctcaatctagggcgaagagaggaatatgacgtcttggagaagaagcgggctcgggatccagagatgggaggcaaacagcagaggaggaggaacccccaggaaggcgtatacaatgcactgcagaaagacaagatggcagaagcctacagtgagatcggcacaaaaggcgagaggcggagaggcaaggggcacgatggcctttaccagggtctcagcactgccaccaaggacacctatgatgccctgcatatgcagaccctggcccctcgctaa

SEQ ID NO: 25 - protein, 517 aa, synthetic construct
  REGION 1..517: note = FcgRI-CD3zeta CONSTRUCT AA SEQUENCE
MILTSFGDDMWLLTTLLLWVPVGGEVVNATKAVITLQPPWVSIFQKENVTLWCEGPHLPGDSSTQWFINGTAVQISTPSYSIPEASFQDSGEYRCQIGSSMPSDPVQLQIHNDWLLLQASRRVLTEGEPLALRCHGWKNKLVYNVVFYRNGKSFQFSSDSEVAILKTNLSHSGIYHCSGTGRHRYTSAGVSITVKELFTTPVLRASVSSPFPEGSLVTLNCETNLLLQRPGLQLHFSFYVGSKILEYRNTSSEYHIARAEREDAGFYWCEVATEDSSVLKRSPELELQVLGPQSSAPVWFHILFYLSVGIMFSLNTVLYVKIHRLQREKKYNLEVPLVSEQGKKANSFQQVRSDGVYEEVTATASQTTPKEAPDGPRSSVGDCGPEQPEPLPPSDSTGAQTSQSRAKFSRSAETAANLQDPNQLYNELNLGRREEYDVLEKKRARDPEMGGKQQRRRNPQEGVYNALQKDKMAEAYSEIGTKGERRRGKGHDGLYQGLSTATKDTYDALHMQTLAPR

SEQ ID NO: 26 - DNA, 1554 bp, synthetic construct
  misc_feature 1..1554: note = FcgRI-CD3zeta CONSTRUCT NA SEQUENCE
  source 1..1554: mol_type = other DNA
atgattcttaccagctttggagatgacatgtggcttctaacaactctgctactttgggttccagtcggtggggaagtggttaatgccaccaaggctgtgatcaccttgcagcctccatgggtcagtattttccagaaggaaaatgtcactttatggtgtgaggggcctcacctgcctggagacagttccacacaatggtttatcaacggaacagccgttcagatctccacgcctagttatagcatcccagaggccagttttcaggacagtggcgaatacaggtgtcagataggttcctcaatgccaagtgaccctgtgcagttgcaaatccacaatgattggctgctactccaggcctcccgcagagtcctcacagaaggagaacccctggccttgaggtgtcacggatggaagaataaactggtgtacaatgtggttttctatagaaatggaaaatcctttcagttttcttcagattcggaggtcgccattctgaaaaccaacctgagtcacagcggcatctaccactgctcaggcacgggaagacaccgctacacatctgcaggagtgtccatcacggtgaaagagctgtttaccacgccagtgctgagagcatccgtgtcatctcccttcccggaggggagtctggtcaccctgaactgtgagacgaatttgctcctgcagagacccggcttacagcttcacttctccttctacgtgggcagcaagatcctggagtacaggaacacatcctcagagtaccatatagcaagggcggaaagagaagatgctggattctactggtgtgaggtagccacggaggacagcagtgtccttaagcgcagccctgagttggagctccaagtgcttggtccccagtcatcagctcctgtctggtttcacatcctgttttatctgtcagtgggaataatgttttcgttgaacacggttctctatgtgaaaatacacaggctgcagagagagaagaaatacaacttagaagtccctttggtttctgagcagggaaagaaagcaaattcctttcagcaagttagaagcgatggcgtgtatgaagaagtaacagccactgcgagccagaccacaccaaaagaagcgcccgatggacctcgaagctcagtgggtgactgtggacccgagcagcctgaaccccttcctcccagtgacagtactggggcacaaacttcccaaagtagagcaaaattcagcaggagtgcagagactgctgccaacctgcaggaccccaaccagctctacaatgagctcaatctagggcgaagagaggaatatgacgtcttggagaagaagcgggctcgggatccagagatgggaggcaaacagcagaggaggaggaacccccaggaaggcgtatacaatgcactgcagaaagacaagatggcagaagcctacagtgagatcggcacaaaaggcgagaggcggagaggcaaggggcacgatggcctttaccagggtctcagcactgccaccaaggacacctatgatgccctgcatatgcagaccctggcccctcgctaa

SEQ ID NO: 27 - protein, 621 aa, synthetic construct
  REGION 1..621: note = FcgRI-CD3zeta-T2A-FcRg CONSTRUCT AA SEQUENCE
MILTSFGDDMWLLTTLLLWVPVGGEVVNATKAVITLQPPWVSIFQKENVTLWCEGPHLPGDSSTQWFINGTAVQISTPSYSIPEASFQDSGEYRCQIGSSMPSDPVQLQIHNDWLLLQASRRVLTEGEPLALRCHGWKNKLVYNVVFYRNGKSFQFSSDSEVAILKTNLSHSGIYHCSGTGRHRYTSAGVSITVKELFTTPVLRASVSSPFPEGSLVTLNCETNLLLQRPGLQLHFSFYVGSKILEYRNTSSEYHIARAEREDAGFYWCEVATEDSSVLKRSPELELQVLGPQSSAPVWFHILFYLSVGIMFSLNTVLYVKIHRLQREKKYNLEVPLVSEQGKKANSFQQVRSDGVYEEVTATASQTTPKEAPDGPRSSVGDCGPEQPEPLPPSDSTGAQTSQSRAKFSRSAETAANLQDPNQLYNELNLGRREEYDVLEKKRARDPEMGGKQQRRRNPQEGVYNALQKDKMAEAYSEIGTKGERRRGKGHDGLYQGLSTATKDTYDALHMQTLAPREGRGSLLTCGDVEENPGPMISAVILFLLLLVEQAAALGEPQLCYILDAVLFLYGIVLTLLYCRLKIQVRKAAIASREKADAVYTGLNTRSQETYETLKHEKPPQ

SEQ ID NO: 28 - DNA, 1866 bp, synthetic construct
  misc_feature 1..1866: note = FcgRI-CD3zeta-T2A-FcRg CONSTRUCT NA SEQUENCE
  source 1..1866: mol_type = other DNA
atgattcttaccagctttggagatgacatgtggcttctaacaactctgctactttgggttccagtcggtggggaagtggttaatgccaccaaggctgtgatcaccttgcagcctccatgggtcagtattttccagaaggaaaatgtcactttatggtgtgaggggcctcacctgcctggagacagttccacacaatggtttatcaacggaacagccgttcagatctccacgcctagttatagcatcccagaggccagttttcaggacagtggcgaatacaggtgtcagataggttcctcaatgccaagtgaccctgtgcagttgcaaatccacaatgattggctgctactccaggcctcccgcagagtcctcacagaaggagaacccctggccttgaggtgtcacggatggaagaataaactggtgtacaatgtggttttctatagaaatggaaaatcctttcagttttcttcagattcggaggtcgccattctgaaaaccaacctgagtcacagcggcatctaccactgctcaggcacgggaagacaccgctacacatctgcaggagtgtccatcacggtgaaagagctgtttaccacgccagtgctgagagcatccgtgtcatctcccttcccggaggggagtctggtcaccctgaactgtgagacgaatttgctcctgcagagacccggcttacagcttcacttctccttctacgtgggcagcaagatcctggagtacaggaacacatcctcagagtaccatatagcaagggcggaaagagaagatgctggattctactggtgtgaggtagccacggaggacagcagtgtccttaagcgcagccctgagttggagctccaagtgcttggtccccagtcatcagctcctgtctggtttcacatcctgttttatctgtcagtgggaataatgttttcgttgaacacggttctctatgtgaaaatacacaggctgcagagagagaagaaatacaacttagaagtccctttggtttctgagcagggaaagaaagcaaattcctttcagcaagttagaagcgatggcgtgtatgaagaagtaacagccactgcgagccagaccacaccaaaagaagcgcccgatggacctcgaagctcagtgggtgactgtggacccgagcagcctgaaccccttcctcccagtgacagtactggggcacaaacttcccaaagtagagcaaaattcagcaggagtgcagagactgctgccaacctgcaggaccccaaccagctctacaatgagctcaatctagggcgaagagaggaatatgacgtcttggagaagaagcgggctcgggatccagagatgggaggcaaacagcagaggaggaggaacccccaggaaggcgtatacaatgcactgcagaaagacaagatggcagaagcctacagtgagatcggcacaaaaggcgagaggcggagaggcaaggggcacgatggcctttaccagggtctcagcactgccaccaaggacacctatgatgccctgcatatgcagaccctggcccctcgcgagggcagaggaagtctgctaacatgcggtgacgtcgaggagaatcctggcccaatgatctcagccgtgatcttgttcttgctccttttggtggaacaagcagccgccctgggagagccgcagctctgctatatcctggatgctgtcctgtttttgtatggtattgtccttaccctactctactgtcgactcaagatccaggtccgaaaggcagctatagccagccgtgagaaagcagatgctgtctacacgggcctgaacacccggagccaggagacatatgagactctgaagcatgagaaaccaccccagtag

SEQ ID NO: 29 - protein, 388 aa, synthetic construct
  REGION 1..388: note = FcgRI-FcRg construct AA sequence
MILTSFGDDMWLLTTLLLWVPVGGEVVNATKAVITLQPPWVSIFQKENVTLWCEGPHLPGDSSTQWFINGTAVQISTPSYSIPEASFQDSGEYRCQIGSSMPSDPVQLQIHNDWLLLQASRRVLTEGEPLALRCHGWKNKLVYNVVFYRNGKSFQFSSDSEVAILKTNLSHSGIYHCSGTGRHRYTSAGVSITVKELFTTPVLRASVSSPFPEGSLVTLNCETNLLLQRPGLQLHFSFYVGSKILEYRNTSSEYHIARAEREDAGFYWCEVATEDSSVLKRSPELELQVLGPQSSAPVWFHILFYLSVGIMFSLNTVLYVLGEPQLCYILDAVLFLYGIVLTLLYCRLKIQVRKAAIASREKADAVYTGLNTRSQETYETLKHEKPPQ

SEQ ID NO: 30 - DNA, 1167 bp, synthetic construct
  misc_feature 1..1167: note = FcgRI-FcRg construct NA sequence
  source 1..1167: mol_type = other DNA
atgattcttaccagctttggagatgacatgtggcttctaacaactctgctactttgggttccagtcggtggggaagtggttaatgccaccaaggctgtgatcaccttgcagcctccatgggtcagtattttccagaaggaaaatgtcactttatggtgtgaggggcctcacctgcctggagacagttccacacaatggtttatcaacggaacagccgttcagatctccacgcctagttatagcatcccagaggccagttttcaggacagtggcgaatacaggtgtcagataggttcctcaatgccaagtgaccctgtgcagttgcaaatccacaatgattggctgctactccaggcctcccgcagagtcctcacagaaggagaacccctggccttgaggtgtcacggatggaagaataaactggtgtacaatgtggttttctatagaaatggaaaatcctttcagttttcttcagattcggaggtcgccattctgaaaaccaacctgagtcacagcggcatctaccactgctcaggcacgggaagacaccgctacacatctgcaggagtgtccatcacggtgaaagagctgtttaccacgccagtgctgagagcatccgtgtcatctcccttcccggaggggagtctggtcaccctgaactgtgagacgaatttgctcctgcagagacccggcttacagcttcacttctccttctacgtgggcagcaagatcctggagtacaggaacacatcctcagagtaccatatagcaagggcggaaagagaagatgctggattctactggtgtgaggtagccacggaggacagcagtgtccttaagcgcagccctgagttggagctccaagtgcttggtccccagtcatcagctcctgtctggtttcacatcctgttttatctgtcagtgggaataatgttttcgttgaacacggttctctatgtgctgggagagccgcagctctgctatatcctggatgctgtcctgtttttgtatggtattgtccttaccctactctactgtcgactcaagatccaggtccgaaaggcagctatagccagccgtgagaaagcagatgctgtctacacgggcctgaacacccggagccaggagacatatgagactctgaagcatgagaaaccaccccagtaa

SEQ ID NO: 31 - protein, 519 aa, synthetic construct
  REGION 1..519: note = FcgRI-FcRg-CD3zeta construct AA sequence
MILTSFGDDMWLLTTLLLWVPVGGEVVNATKAVITLQPPWVSIFQKENVTLWCEGPHLPGDSSTQWFINGTAVQISTPSYSIPEASFQDSGEYRCQIGSSMPSDPVQLQIHNDWLLLQASRRVLTEGEPLALRCHGWKNKLVYNVVFYRNGKSFQFSSDSEVAILKTNLSHSGIYHCSGTGRHRYTSAGVSITVKELFTTPVLRASVSSPFPEGSLVTLNCETNLLLQRPGLQLHFSFYVGSKILEYRNTSSEYHIARAEREDAGFYWCEVATEDSSVLKRSPELELQVLGPQSSAPVWFHILFYLSVGIMFSLNTVLYVMISAVILFLLLLVEQAAALGEPQLCYILDAVLFLYGIVLTLLYCRLKIQVRKAAIASREKADAVYTGLNTRSQETYETLKHEKPPQRAKFSRSAETAANLQDPNQLYNELNLGRREEYDVLEKKRARDPEMGGKQQRRRNPQEGVYNALQKDKMAEAYSEIGTKGERRRGKGHDGLYQGLSTATKDTYDALHMQTLAPR

SEQ ID NO: 32 - DNA, 1560 bp, synthetic construct
  misc_feature 1..1560: note = FcgRI-FcRg-CD3zeta construct NA sequence
  source 1..1560: mol_type = other DNA
atgattcttaccagctttggagatgacatgtggcttctaacaactctgctactttgggttccagtcggtggggaagtggttaatgccaccaaggctgtgatcaccttgcagcctccatgggtcagtattttccagaaggaaaatgtcactttatggtgtgaggggcctcacctgcctggagacagttccacacaatggtttatcaacggaacagccgttcagatctccacgcctagttatagcatcccagaggccagttttcaggacagtggcgaatacaggtgtcagataggttcctcaatgccaagtgaccctgtgcagttgcaaatccacaatgattggctgctactccaggcctcccgcagagtcctcacagaaggagaacccctggccttgaggtgtcacggatggaagaataaactggtgtacaatgtggttttctatagaaatggaaaatcctttcagttttcttcagattcggaggtcgccattctgaaaaccaacctgagtcacagcggcatctaccactgctcaggcacgggaagacaccgctacacatctgcaggagtgtccatcacggtgaaagagctgtttaccacgccagtgctgagagcatccgtgtcatctcccttcccggaggggagtctggtcaccctgaactgtgagacgaatttgctcctgcagagacccggcttacagcttcacttctccttctacgtgggcagcaagatcctggagtacaggaacacatcctcagagtaccatatagcaagggcggaaagagaagatgctggattctactggtgtgaggtagccacggaggacagcagtgtccttaagcgcagccctgagttggagctccaagtgcttggtccccagtcatcagctcctgtctggtttcacatcctgttttatctgtcagtgggaataatgttttcgttgaacacggttctctatgtgatgatctcagccgtgatcttgttcttgctccttttggtggaacaagcagccgccctgggagagccgcagctctgctatatcctggatgctgtcctgtttttgtatggtattgtccttaccctactctactgtcgactcaagatccaggtccgaaaggcagctatagccagccgtgagaaagcagatgctgtctacacgggcctgaacacccggagccaggagacatatgagactctgaagcatgagaaaccaccccagagagcaaaattcagcaggagtgcagagactgctgccaacctgcaggaccccaaccagctctacaatgagctcaatctagggcgaagagaggaatatgacgtcttggagaagaagcgggctcgggatccagagatgggaggcaaacagcagaggaggaggaacccccaggaaggcgtatacaatgcactgcagaaagacaagatggcagaagcctacagtgagatcggcacaaaaggcgagaggcggagaggcaaggggcacgatggcctttaccagggtctcagcactgccaccaaggacacctatgatgccctgcatatgcagaccctggcccctcgctaa

SEQ ID NO: 33 - protein, 23 aa, synthetic construct
  REGION 1..23: note = Transmembrane+intracellular domain of CD64 AA sequence
VWFHILFYLSVGIMFSLNTVLYV

SEQ ID NO: 34 - protein, 296 aa, synthetic construct
  REGION 1..296: note = Extracellular +transmembrane domain of CD64 AA sequence
EVVNATKAVITLQPPWVSIFQKENVTLWCEGPHLPGDSSTQWFINGTAVQISTPSYSIPEASFQDSGEYRCQIGSSMPSDPVQLQIHNDWLLLQASRRVLTEGEPLALRCHGWKNKLVYNVVFYRNGKSFQFSSDSEVAILKTNLSHSGIYHCSGTGRHRYTSAGVSITVKELFTTPVLRASVSSPFPEGSLVTLNCETNLLLQRPGLQLHFSFYVGSKILEYRNTSSEYHIARAEREDAGFYWCEVATEDSSVLKRSPELELQVLGPQSSAPVWFHILFYLSVGIMFSLNTVLYV

SEQ ID NO: 35 - protein, 591 aa, synthetic construct
  REGION 1..591: note = TRP-1 CD4 TCR (alpha chain, P2A, beta chain)Construct, AA sequence
MVLALLPVLGIHFLLRDAQAQSVTQPDARVTVSEGASLQLRCKYSSSVTPYLFWYVQYPRQGLQLLLKYYSGDPVVQGVNGFEAEFSKSNSSFHLRKASVHWSDSAVYFCAVSSNNNRIFFGDGTQLVVKPNIQNPEPAVYQLKDPRSQDSTLCLFTDFDSQINVPKTMESGTFITDKTVLDMKAMDSKSNGAIAWSNQTSFTCQDIFKETNATYPSSDVPCDATLTEKSFETDMNLNFQNLSVMGLRILLLKVAGFNLLMTLRLWSSGSGATNFSLLKQAGDVQENPGPLYSLLAFLLGMFLGVSAQTIHQWPVAEIKAVGSPLSLGCTIKGKSSPNLYWYWQATGGTLQQLFYSITVGQVESVVQLNLSASRPKDDQFILSTEKLLLSHSGFYLCAWSPGHQDTQYFGPGTRLLVLEDLRNVTPPKVSLFEPSKAEIANKQKATLVCLARGFFPDHVELSWWVNGKEVHSGVSTDPQAYKESNYSYCLSSRLRVSATFWHNPRNHFRCQVQFHGLSEEDKWPEGSPKPVTQNISAEAWGRADCGITSASYQQGVLSATILYEILLGKATLYAVLVSTLVVMAMVKRKNS

SEQ ID NO: 36 - DNA, 1776 bp, synthetic construct
  misc_feature 1..1776: note = TRP-1 CD4 TCR (alpha chain, P2A, beta chain)Construct, NA sequence
  source 1..1776: mol_type = other DNA
atggtcctggcgctcctcccagtgctggggatacactttctcctgagagatgcccaagctcagtcagtgacgcagcccgatgctcgtgtcactgtctctgaaggagcctctctgcagctgagatgcaagtattcctcctctgtgacaccttatctgttctggtatgtccagtacccgcggcaggggctgcagctgctcctcaagtactattccggagacccagtggttcaaggagtgaatggctttgaggctgagttcagcaagagtaactcttccttccacctgcggaaagcctccgtgcactggagcgactcggctgtgtacttctgtgctgtgagctcgaacaataacagaatcttctttggtgatgggacgcagctggtggtgaagcccaacatccagaacccagaacctgctgtgtaccagttaaaagatcctcggtctcaggacagcaccctctgcctgttcaccgactttgactcccaaatcaatgtgccgaaaactatggaatctggaacgttcatcactgacaaaactgtgctggacatgaaagctatggattccaagagcaatggggccattgcctggagcaaccagacaagcttcacctgccaagatatcttcaaagagaccaacgccacctaccccagttcagacgttccctgtgatgccacgttgactgagaaaagctttgaaacagatatgaacctaaactttcaaaacctgtcagttatgggactccgaatcctcctgctgaaagtagccggatttaacctgctcatgacgctgaggctgtggtccagtggctctggcgccacgaacttctctctgttaaagcaagcaggagacgtgcaagaaaaccccggtcccctgtactctctccttgcctttctcctgggcatgttcttgggtgttagtgctcagactatccatcaatggccagttgccgagatcaaggctgtgggcagcccactgtctctggggtgtaccataaaggggaaatcaagccctaacctctactggtactggcaggccacaggaggcaccctccagcaactcttctactctattactgttggccaggtagagtcggtggtgcaactgaacctctcagcttccaggccgaaggacgaccaattcatcctaagcacggagaagctgcttctcagccactctggcttctacctctgtgcctggagtccgggacatcaagacacccagtactttgggccaggcactcggctcctcgtgttagaggatctgagaaatgtgactccacccaaggtctccttgtttgagccatcaaaagcagagattgcaaacaaacaaaaggctaccctcgtgtgcttggccaggggcttcttccctgaccacgtggagctgagctggtgggtgaatggcaaggaggtccacagtggggtcagcacggaccctcaggcctacaaggagagcaattatagctactgcctgagcagccgcctgagggtctctgctaccttctggcacaatcctcgcaaccacttccgctgccaagtgcagttccacgggctttcagaggaggacaagtggccagagggctcacccaaacctgtcacacagaacatcagtgcagaggcctggggccgagcagactgtgggattacctcagcatcctatcaacaaggggtcttgtctgccaccatcctctatgagatcctgctagggaaagccaccctgtatgctgtgcttgtcagtacactggtggtgatggctatggtcaaaagaaagaattcatga

SEQ ID NO: 37 - protein, 172 aa, synthetic construct
  REGION 1..172: note = TCR-betta constant region aa sequence
DLRNVTPPKVSLFEPSKAEIANKQKATLVCLARGFFPDHVELSWWVNGKEVHSGVSTDPQAYKESNYSYCLSSRLRVSATFWHNPRNHFRCQVQFHGLSEEDKWPEGSPKPVTQNISAEAWGRADCGITSASYHQGVLSATILYEILLGKATLYAVLVSGLVLMAMVKRKNS

SEQ ID NO: 38 - DNA, 516 bp, synthetic construct
  misc_feature 1..516: note = TCR-betta constant region NA sequence
  source 1..516: mol_type = other DNA
gatctgagaaatgtgactccacccaaggtctccttgtttgagccatcaaaagcagagattgcaaacaaacaaaaggctaccctcgtgtgcttggccaggggcttcttccctgaccacgtggagctgagctggtgggtgaatggcaaggaggtccacagtggggtcagcacggaccctcaggcctacaaggagagcaattatagctactgcctgagcagccgcctgagggtctctgctaccttctggcacaatcctcgaaaccacttccgctgccaagtgcagttccatgggctttcggaggaggacaagtggccagagggctcacccaaacctgtcacacagaacatcagtgcagaggcctggggccgagcagactgtggaatcacttcagcatcctatcatcagggggttctgtctgcaaccatcctctatgagatcctactggggaaggccaccctatatgctgtgctggtcagtggcctggtgctgatggccatggtcaagagaaaaaattcc

SEQ ID NO: 39 - protein, 66 aa, synthetic construct
  REGION 1..66: note = CD8 Hinge aa sequence
TTTKPVLRTPSPVHPTGTSQPQRPEDCRPRGSVKGTGLDFACDIYIWAPLAGICVALLLSLIITLI

SEQ ID NO: 40 - DNA, 198 bp, synthetic construct
  misc_feature 1..198: note = CD8 Hinge NA sequence
  source 1..198: mol_type = other DNA
actactaccaagccagtgctgcgaactccctcacctgtgcaccctaccgggacatctcagccccagagaccagaagattgtcggccccgtggctcagtgaaggggaccggattggacttcgcctgtgatatttacatctgggcacccttggccggaatctgcgtggcccttctgctgtccttgatcatcactctcatc

SEQ ID NO: 41 - protein, 382 aa, synthetic construct
  REGION 1..382: note = FcgRI extracellular-TCRbeta constant region construct aa sequence
MILTSFGDDMWLLTTLLLWVPVGGEVVNATKAVITLQPPWVSIFQKENVTLWCEGPHLPGDSSTQWFINGTAVQISTPSYSIPEASFQDSGEYRCQIGSSMPSDPVQLQIHNDWLLLQASRRVLTEGEPLALRCHGWKNKLVYNVVFYRNGKSFQFSSDSEVAILKTNLSHSGIYHCSGTGRHRYTSAGVSITVKELFTTPVLRGSTRPRDLRNVTPPKVSLFEPSKAEIANKQKATLVCLARGFFPDHVELSWWVNGKEVHSGVSTDPQAYKESNYSYCLSSRLRVSATFWHNPRNHFRCQVQFHGLSEEDKWPEGSPKPVTQNISAEAWGRADCGITSASYHQGVLSATILYEILLGKATLYAVLVSGLVLMAMVKRKNS

SEQ ID NO: 42 - DNA, 1146 bp, synthetic construct
  misc_feature 1..1146: note = FcgRI extracellular-TCRbeta constant region construct NA sequence
  source 1..1146: mol_type = other DNA
atgattcttaccagctttggagatgacatgtggcttctaacaactctgctactttgggttccagtcggtggggaagtggttaatgccaccaaggctgtgatcaccttgcagcctccatgggtcagtattttccagaaggaaaatgtcactttatggtgtgaggggcctcacctgcctggagacagttccacacaatggtttatcaacggaacagccgttcagatctccacgcctagttatagcatcccagaggccagttttcaggacagtggcgaatacaggtgtcagataggttcctcaatgccaagtgaccctgtgcagttgcaaatccacaatgattggctgctactccaggcctcccgcagagtcctcacagaaggagaacccctggccttgaggtgtcacggatggaagaataaactggtgtacaatgtggttttctatagaaatggaaaatcctttcagttttcttcagattcggaggtcgccattctgaaaaccaacctgagtcacagcggcatctaccactgctcaggcacgggaagacaccgctacacatctgcaggagtgtccatcacggtgaaagagctgtttaccacgccagtgctgagaggatccacgcgtccgcgggatctgagaaatgtgactccacccaaggtctccttgtttgagccatcaaaagcagagattgcaaacaaacaaaaggctaccctcgtgtgcttggccaggggcttcttccctgaccacgtggagctgagctggtgggtgaatggcaaggaggtccacagtggggtcagcacggaccctcaggcctacaaggagagcaattatagctactgcctgagcagccgcctgagggtctctgctaccttctggcacaatcctcgaaaccacttccgctgccaagtgcagttccatgggctttcggaggaggacaagtggccagagggctcacccaaacctgtcacacagaacatcagtgcagaggcctggggccgagcagactgtggaatcacttcagcatcctatcatcagggggttctgtctgcaaccatcctctatgagatcctactggggaaggccaccctatatgctgtgctggtcagtggcctggtgctgatggccatggtcaagagaaaaaattcc

SEQ ID NO: 43 - protein, 405 aa, synthetic construct
  REGION 1..405: note = FcgRI extracellular-CD8-hinge+TM-Fcrg construct aa sequence
MILTSFGDDMWLLTTLLLWVPVGGEVVNATKAVITLQPPWVSIFQKENVTLWCEGPHLPGDSSTQWFINGTAVQISTPSYSIPEASFQDSGEYRCQIGSSMPSDPVQLQIHNDWLLLQASRRVLTEGEPLALRCHGWKNKLVYNVVFYRNGKSFQFSSDSEVAILKTNLSHSGIYHCSGTGRHRYTSAGVSITVKELFTTPVLRASVSSPFPEGSLVTLNCETNLLLQRPGLQLHFSFYVGSKILEYRNTSSEYHIARAEREDAGFYWCEVATEDSSVLKRSPELELQVLGPQSSAPTTTKPVLRTPSPVHPTGTSQPQRPEDCRPRGSVKGTGLDFACDIYIWAPLAGICVALLLSLIITLIRLKIQVRKAAIASREKADAVYTGLNTRSQETYETLKHEKPPQ

SEQ ID NO: 44 - DNA, 1218 bp, synthetic construct
  misc_feature 1..1218: note = FcgRI extracellular-CD8-hinge+TM-Fcrg construct NA sequence
  source 1..1218: mol_type = other DNA
atgattcttaccagctttggagatgacatgtggcttctaacaactctgctactttgggttccagtcggtggggaagtggttaatgccaccaaggctgtgatcaccttgcagcctccatgggtcagtattttccagaaggaaaatgtcactttatggtgtgaggggcctcacctgcctggagacagttccacacaatggtttatcaacggaacagccgttcagatctccacgcctagttatagcatcccagaggccagttttcaggacagtggcgaatacaggtgtcagataggttcctcaatgccaagtgaccctgtgcagttgcaaatccacaatgattggctgctactccaggcctcccgcagagtcctcacagaaggagaacccctggccttgaggtgtcacggatggaagaataaactggtgtacaatgtggttttctatagaaatggaaaatcctttcagttttcttcagattcggaggtcgccattctgaaaaccaacctgagtcacagcggcatctaccactgctcaggcacgggaagacaccgctacacatctgcaggagtgtccatcacggtgaaagagctgtttaccacgccagtgctgagagcatccgtgtcatctcccttcccggaggggagtctggtcaccctgaactgtgagacgaatttgctcctgcagagacccggcttacagcttcacttctccttctacgtgggcagcaagatcctggagtacaggaacacatcctcagagtaccatatagcaagggcggaaagagaagatgctggattctactggtgtgaggtagccacggaggacagcagtgtccttaagcgcagccctgagttggagctccaagtgcttggtccccagtcatcagctcctactactaccaagccagtgctgcgaactccctcacctgtgcaccctaccgggacatctcagccccagagaccagaagattgtcggccccgtggctcagtgaaggggaccggattggacttcgcctgtgatatttacatctgggcacccttggccggaatctgcgtggcccttctgctgtccttgatcatcactctcatccgactcaagatccaggtccgaaaggcagctatagccagccgtgagaaagcagatgctgtctacacgggcctgaacacccggagccaggagacatatgagactctgaagcatgagaaaccaccccagtag

SEQ ID NO: 45 - protein, 36 aa, synthetic construct
  REGION 1..36: note = OX40 intracellular domain AA sequence
RKAWRLPNTPKPCWGNSFRTPIQEEHTDAHFTLAKI

SEQ ID NO: 46 - DNA, 108 bp, synthetic construct
  misc_feature 1..108: note = OX40 intracellular domain NA sequence
  source 1..108: mol_type = other DNA
cggaaggcttggagattgcctaacactcccaaaccttgttggggaaacagcttcaggaccccgatccaggaggaacacacagacgcacactttactctggccaagatc

SEQ ID NO: 47 - protein, 48 aa, synthetic construct
  REGION 1..48: note = 41BB intracellular domain AA sequence
SVLKWIRKKFPHIFKQPFKKTTGAAQEEDACSCRCPQEEEGGGGGYEL

SEQ ID NO: 48 - DNA, 144 bp, synthetic construct
  misc_feature 1..144: note = 41BB intracellular domain NA sequence
  source 1..144: mol_type = other DNA
tctgtgctcaaatggatcaggaaaaaattcccccacatattcaagcaaccatttaagaagaccactggagcagctcaagaggaagatgcttgtagctgccgatgtccacaggaagaagaaggaggaggaggaggctatgagctg

SEQ ID NO: 49 - protein, 186 aa, synthetic construct
  REGION 1..186: note = TNFR2 intracellular signaling domain AA SEQUENCE
KKKPSCLQRDAKVPHVPDEKSQDAVGLEQQHLLTTAPSSSSSSLESSASAGDRRAPPGGHPQARVMAEAQGFQEARASSRISDSSHGSHGTHVNVTCIVNVCSSSDHSSQCSSQASATVGDPDAKPSASPKDEQVPFSQEECPSQSPCETTETLQSHEKPLPLGVPDMGMKPSQAGWFDQIAVKVA

SEQ ID NO: 50 - DNA, 558 bp, synthetic construct
  misc_feature 1..558: note = TNFR2 intracellular signaling domain NA SEQUENCE
  source 1..558: mol_type = other DNA
aaaaagaagccctcctgcctacaaagagatgccaaggtgcctcatgtgcctgatgagaaatcccaggatgcagtaggccttgagcagcagcacctgttgaccacagcacccagttccagcagcagctccctagagagctcagccagcgctggggaccgaagggcgccccctgggggccatccccaagcaagagtcatggcggaggcccaagggtttcaggaggcccgtgccagctccaggatttcagattcttcccacggaagccacgggacccacgtcaacgtcacctgcatcgtgaacgtctgtagcagctctgaccacagttctcagtgctcttcccaagccagcgccacagtgggagacccagatgccaagccctcagcgtccccaaaggatgagcaggtccccttctctcaggaggagtgtccgtctcagtccccgtgtgagactacagagacactgcagagccatgagaagcccttgccccttggtgtgccggatatgggcatgaagcccagccaagctggctggtttgatcagattgcagtcaaagtggcc

SEQ ID NO: 51 - protein, 147 aa, synthetic construct
  REGION 1..147: note = IL-12Rb1 intracellular AA SEQUENCE
NRAAWHLCPPLPTPCGSTAVEFPGSQGKQAWQWCNPEDFPEVLYPRDALVVEMPGDRGDGTESPQAAPECALDTRRPLETQRQRQVQALSEARRLGLAREDCPRGDLAHVTLPLLLGGVTQGASVLDDLWRTHKTAEPGPPTLGQEA

SEQ ID NO: 52 - DNA, 441 bp, synthetic construct
  misc_feature 1..441: note = IL-12Rb1 intracellular NA SEQUENCE
  source 1..441: mol_type = other DNA
aacagggccgcctggcacttgtgcccacccctgcctacaccctgtggcagcactgccgtggagttccctggcagccagggcaagcaggcttggcagtggtgcaaccctgaggacttcccggaggtgttgtacccgcgagatgcgctggtggtcgagatgcccggagacagaggcgacgggacagagtcgccccaggccgcccctgagtgcgccctggacacaaggcggcccttggagactcagaggcagaggcaggtgcaggcactgtcagaggccaggcgcctgggcctggctagggaggactgtccccgtggtgacctggcccacgtgacactcccgctgctcctgggaggtgtgacccagggagcctctgtacttgacgatctttggaggacccataagactgcggagccgggaccgcccactttggggcaagaggcc

SEQ ID NO: 53 - protein, 216 aa, synthetic construct
  REGION 1..216: note = IL-12Rb2 intracellular AA SEQUENCE
RYFRQKAFTLLSTLKPQWYSRTIPDPANSTWVKKYPILEEKIQLPTDNLLMAWPTPEEPEPLIIHEVLYHMIPVVRQPYYFKRGQGFQGYSTSKQDAMYIANPQATGTLTAETRQLVNLYKVLESRDPDSKLANLTSPLTVTPVNYLPSHEGYLPSNIEDLSPHEADPTDSFDLEHQHISLSIFASSSLRPLIFGGERLTLDRLKMGYDSLMSNEA

SEQ ID NO: 54 - DNA, 648 bp, synthetic construct
  misc_feature 1..648: note = IL-12Rb2 intracellular NA SEQUENCE
  source 1..648: mol_type = other DNA
cgttacttccggcaaaaggcatttactctcctgtctactctcaaacctcaatggtatagcagaaccattccagatccagcaaacagcacttgggtaaagaagtatcccattctggaggagaagatccagctacctacggataatctcctgatggcatggcccactcctgaagagcctgagcccctgatcatccatgaagtcctctaccacatgatcccagttgtcagacaaccatattacttcaaaagaggccaaggattccaaggctactctacctccaagcaagatgcaatgtatattgccaatccacaagctacaggaactctcacagctgagaccagacagctagtgaacctatacaaggtgctagaaagcagagaccctgactcaaaactggccaacctgaccagccccttgacagtcaccccagtgaactaccttcctagccatgaaggctatttaccctccaacatagaagatctgtcaccacatgaggctgacccaactgattcttttgacctggagcatcaacatatttctctttccatttttgcatcaagttctctccgcccactcatcttcggtggtgagcggctgactctagatcggttaaagatgggctatgactccctcatgagtaatgaggct

SEQ ID NO: 55 - protein, 249 aa, synthetic construct
  REGION 1..249: note = IL-23R intracellular AA SEQUENCE
NRSLRIGIKRKVLLMIPKWLYEDIPNMENSNVAKLLQEKSVFENDNASEQALYVDPVLTEISEISPLEHKPTDYKEERLTGLLETRDCPLGMLSTSSSVVYIPDLNTGYKPQVSNVPPGGNLFINRDERDPTSLETTDDHFARLKTYPNFQFSASSMALLNKTLILDELCLVLNQGEFNSLDIKNSRQEETSIVLQSDSPSETIPAQTLLSDEFVSCLAIGNEDLPSINSYFPQNVLESHFSRISLFQK

SEQ ID NO: 56 - DNA, 747 bp, synthetic construct
  misc_feature 1..747: note = IL-23R intracellular NA SEQUENCE
  source 1..747: mol_type = other DNA
aacagatcacttcgaataggaattaaaagaaaagttttactgatgatcccaaagtggctttatgaagatattcctaatatggaaaatagcaatgttgcaaaattattacaggaaaaaagtgtatttgagaatgataatgccagtgagcaggccctgtatgtggatcctgtccttacagagataagtgaaatctctcccctggaacacaaacccacagattacaaagaagaaaggctcacaggactccttgagacaagagactgtcctctaggaatgttgtctaccagttcttctgttgtgtatattcctgacctcaacactggatacaaaccccaggtttcaaatgttcctcctggaggaaaccttttcattaacagagatgaaagagaccctacatcccttgagaccacagatgaccactttgccagattgaaaacatatcccaacttccaattttctgcttcaagtatggctttactaaacaaaacactaattcttgatgaattgtgcctcgttttaaatcaaggagagttcaattctcttgacataaaaaactcaagacaggaggaaaccagcatcgttttgcaaagtgactcacccagtgaaactatcccagcgcagactctgttgtctgatgaatttgtctcctgtttggcaattgggaatgaagacttgccatctattaattcttactttccacagaacgttttggaaagccatttcagtagaatttcactcttccaaaag

SEQ ID NO: 57 - protein, 202 aa, synthetic construct
  REGION 1..202: note = IFNgR1 intracellular AA SEQUENCE
WYTKNNSFKRKSIMLPKSLLSVVKSATLETKPESKYSLVTPHQPAVLESETVICEEPLSTVTAPDSPEAAEQEELSKETKALEAGGSTSAMTPDSPPTPTQRRSFSLLSSNQSGPCSLTAYHSRNGSDSGLVGSGSSISDLESLPNNNSETKMAEHDPPPVRKAPMASGYDKPHMLVDVLVDVGGKESLMGYRLTGEAQELS

SEQ ID NO: 58 - DNA, 606 bp, synthetic construct
  misc_feature 1..606: note = IFNgR1 intracellular NA SEQUENCE
  source 1..606: mol_type = other DNA
tggtatactaagaacaattcattcaagagaaaaagcataatgttacctaagtccttgctctctgtggtaaaaagtgccacgttagagacaaaacctgaatcgaagtattcacttgtcacaccgcaccagccagctgtcctagagagtgagacggtgatctgtgaagagcccctgtccacagtgacagctccagacagccccgaagcagcagaacaggaagaactttcaaaagaaacaaaggctctggaggctggaggaagcacgtctgccatgaccccagacagccctccaactccgacacaaagacgcagcttttccctgttaagtagtaaccagtcaggcccttgtagcctcaccgcctatcactcccgaaacggctctgacagtggcctcgtgggatcgggcagctccatatcggacttggaatctctcccaaacaacaactcagaaacaaagatggcagagcacgaccctccacccgtgagaaaggcccccatggcctccggttatgacaaaccgcacatgttggtggacgtgcttgtggatgttggggggaaggagtctctcatggggtatagactcacaggagaggcccaggagctgtcc

SEQ ID NO: 59 - protein, 67 aa, synthetic construct
  REGION 1..67: note = IFNgR2 intracellular AA SEQUENCE
LKYQSRVKYWFQAPPNIPEQIEEYLKDPDQFILEVLDKDGSPKEDSWDSVSIISSPEKERDDVLQTP

SEQ ID NO: 60 - DNA, 201 bp, synthetic construct
  misc_feature 1..201: note = IFNgR2 intracellular NA SEQUENCE
  source 1..201: mol_type = other DNA
ctcaaataccaaagccgagtgaagtactggtttcaggctccgccaaacatcccggaacaaatcgaagagtatctaaaggacccagaccaattcatcttagaggtcttggacaaggacggttcaccgaaggaggactcctgggactccgtgtcaattatttcttctccagaaaaggagcgagatgatgtgctccaaacaccg

SEQ ID NO: 61 - protein, 271 aa, synthetic construct
  REGION 1..271: note = IL-2Rb intracellular AA SEQUENCE
KCRYLGPWLKTVLKCHIPDPSEFFSQLSSQHGGDLQKWLSSPVPLSFFSPSGPAPEISPLEVLDGDSKAVQLLLLQKDSAPLPSPSGHSQASCFTNQGYFFFHLPNALEIESCQVYFTYDPCVEEEVEEDGSRLPEGSPHPPLLPLAGEQDDYCAFPPRDDLLLFSPSLSTPNTAYGGSRAPEERSPLSLHEGLPSLASRDLMGLQRPLERMPEGDGEGLSANSSGEQASVPEGNLHGQDQDRGQGPILTLNTDAYLSLQELQAQDSVHLI

SEQ ID NO: 62 - DNA, 813 bp, synthetic construct
  misc_feature 1..813: note = IL-2Rb intracellular NA SEQUENCE
  source 1..813: mol_type = other DNA
aagtgccggtaccttgggccatggctgaagacagttctcaagtgccacatcccagatccttctgagttcttctcccagctgagctcccagcatgggggagaccttcagaaatggctctcctcgcctgtccccttgtccttcttcagccccagtggccctgcccctgagatctctccgctggaagtgctcgacggagattccaaggccgtgcagctgctcctgttacagaaggactctgcccctttaccctcgcccagcggccactcacaggccagctgcttcaccaaccagggctacttcttcttccatctgcccaatgccttggagatcgaatcctgccaggtgtacttcacctatgacccctgtgtggaagaggaggtggaggaggatgggtcaaggctgcccgagggatctccccacccacctctgctgcctctggctggagaacaggatgactactgtgccttcccgcccagggatgacctgctgctcttctccccgagcctcagcacccccaacactgcctatgggggcagcagagcccctgaagaaagatctccactctccctgcatgagggacttccctccctagcatcccgtgacctgatgggcttacagcgccctctggagcggatgccggaaggtgatggagaggggctgtctgccaatagctctggggagcaggccagtgtcccagaaggcaaccttcatgggcaagatcaggacagaggccagggccccatcctgaccctgaacaccgatgcctatctgtctcttcaagaactacaggcccaagattcagtccacctaata

SEQ ID NO: 63 - protein, 85 aa, synthetic construct
  REGION 1..85: note = IL-2R common gamma intracellular AA sequence
ERMPPIPPIKNLEDLVTEYQGNFSAWSGVSKGLTESLQPDYSERFCHVSEIPPKGGALGEGPGGSPCSLHSPYWPPPCYSLKPEA

SEQ ID NO: 64 - DNA, 255 bp, synthetic construct
  misc_feature 1..255: note = IL-2R common gamma intracellular NA sequence
  source 1..255: mol_type = other DNA
gaacgaatgcctccaattccccccatcaagaatctagaggatctggttactgaataccaagggaacttttcggcctggagtggtgtgtctaaagggctgactgagagtctgcagccagactacagtgaacggttctgccacgtcagcgagattccccccaaaggaggggccctaggagaggggcctggaggttctccttgcagcctgcatagcccttactggcctcccccatgttattctctgaagccggaagcc

SEQ ID NO: 65 - protein, 217 aa, synthetic construct
  REGION 1..217: note = IL-1R1 intracellular AA sequence
KVFKVDIVLWYRDSCSGFLPSKASDGKTYDAYILYPKTLGEGSFSDLDTFVFKLLPEVLEGQFGYKLFIYGRDDYVGEDTIEVTNENVKKSRRLIIILVRDMGGFSWLGQSSEEQIAIYNALIQEGIKIVLLELEKIQDYEKMPDSIQFIKQKHGVICWSGDFQERPQSAKTRFWKNLRYQMPAQRRSPLSKHRLLTLDPVRDTKEKLPAATHLPLG

SEQ ID NO: 66 - DNA, 651 bp, synthetic construct
  misc_feature 1..651: note = IL-1R1 intracellular NA sequence
  source 1..651: mol_type = other DNA
aaagtcttcaaggttgacatagtgctttggtacagggactcctgctctggttttcttccttcaaaagcttcagatggaaagacatacgatgcctatattctttatcccaagaccctgggagaggggtccttctcagacttagatacttttgtttttaaactgttgcctgaggtcttggagggacagtttggatacaagctgttcatttatggaagggatgactatgttggagaagataccatcgaggttactaatgaaaatgtaaagaaaagcaggaggctgattatcattctagtgagagatatgggaggcttcagctggctgggccagtcatctgaagagcaaatagccatatacaatgctctcatccaggaaggaattaaaatcgtcctgcttgagttggagaaaatccaagactatgagaaaatgccagattctattcagttcattaagcagaaacacggagtcatttgctggtcaggagactttcaagaaagaccacagtctgcaaagaccaggttctggaaaaacttaagataccagatgccagcccaacggagatcaccattgtctaaacaccgcttactaaccctggatcctgtgcgggacactaaggagaaactgccggcagcaacacacttaccactcggc

SEQ ID NO: 67 - protein, 182 aa, synthetic construct
  REGION 1..182: note = IL-1AcP intracellular AA sequence
YRAHFGTDETILDGKEYDIYVSYARNVEEEEFVLLTLRGVLENEFGYKLCIFDRDSLPGGIVTDETLSFIQKSRRLLVVLSPNYVLQGTQALLELKAGLENMASRGNINVILVQYKAVKDMKVKELKRAKTVLTVIKWKGEKSKYPQGRFWKQLQVAMPVKKSPRWSSNDKQGLSYSSLKNV

SEQ ID NO: 68 - DNA, 546 bp, synthetic construct
  misc_feature 1..546: note = IL-1AcP intracellular NA sequence
  source 1..546: mol_type = other DNA
taccgagctcactttggaacagatgaaacaattcttgatggaaaggagtatgatatttatgtttcctatgcaagaaatgtggaagaagaggaatttgtgctgctgacgctgcgtggagttttggagaatgagtttggatacaagctgtgcatcttcgacagagacagcctgcctgggggaattgtcacagatgagaccctgagcttcattcagaaaagcagacgactcctggttgtcctaagtcccaactacgtgctccagggaacacaagccctcctggagctcaaggctggcctagaaaatatggcctcacggggcaacatcaacgtcattttagtgcagtacaaagctgtgaaggacatgaaggtgaaagagctgaagcgggctaagacggtgctcacggtcattaaatggaaaggagagaaatccaagtatcctcagggcaggttctggaagcagttgcaggtggccatgccagtgaagaagagtcccaggtggtctagcaatgacaagcagggtctctcctactcatccctgaaaaacgta

(19) United States
(12) Patent Application Publication
(10) Pub. No.: US 2025/0163128 A1
(43) Pub. Date: May 22, 2025

(54) T CELL EXPRESSING AN FC GAMMA RECEPTOR AND METHODS OF USE THEREOF

(71) Applicant: Ramot at Tel-Aviv University Ltd., Tel-Aviv (IL)

(72) Inventors: Yaron CARMI, Tel Aviv (IL); Peleg RIDER, Omer (IL); Diana RASOULOUNIRIANA, Bat Yam (IL)

(73) Assignee: Ramot at Tel-Aviv University Ltd., Tel-Aviv (IL)

(21) Appl. No.: 19/008,721

(22) Filed: Jan. 3, 2025

Related U.S. Application Data
(63) Continuation of application No. 17/478,930, filed on Sep. 19, 2021, now abandoned.
Application No. 17/478,930 is a continuation of application No. PCT/IL2020/050327, filed on Mar. 19, 2020.
(60) Provisional application No. 62/820,357, filed on Mar. 19, 2019.

(51) Int. Cl. C07K 14/735; A61K 39/395; A61K 40/11; A61K 40/32; A61K 40/42; A61P 35/00; C07K 14/725; C12N 5/0783

(52) U.S. Cl. CPC C07K 14/70535; A61K 39/3955; A61K 40/11; A61K 40/32; A61K 40/42; A61K 40/4245; A61P 35/00; C07K 14/7051; C12N 5/0636; A61K 2239/31; A61K 2239/38; A61K 2239/57; C07K 2319/03

(57) ABSTRACT

A T cell expressing an FC gamma receptor is provided. Accordingly there is provided a T cell genetically engineered to express a first polypeptide comprising an amino acid sequence of an Fc receptor common γ chain (FcRγ), said amino acid sequence is capable of transmitting an activating signal; and a second polypeptide comprising an extracellular ligand-binding domain of an Fcγ receptor capable of binding an Fc ligand and an amino acid sequence capable of recruiting said first polypeptide such that upon binding of said Fc ligand to said extracellular ligand-binding domain of said Fcγ receptor said activating signal is transmitted.

### RELATED APPLICATIONS

This application is a Continuation of U.S. patent application Ser. No. 17/478,930, filed on Sep. 19, 2021, which is a Continuation of PCT Patent Application No. PCT/IL2020/050327 having International Filing Date of Mar. 19, 2020, which claims the benefit of priority under 35 USC § 119(e) of U.S. Provisional Patent Application No. 62/820,357 filed on Mar. 19, 2019. The contents of the above applications are all incorporated by reference as if fully set forth herein in their entirety.

### SEQUENCE LISTING STATEMENT

The XML file, entitled 102550SequenceListing.txt, created on Jan. 3, 2025, comprising 116,629 bytes, submitted concurrently with the filing of this application is incorporated herein by reference.

### FIELD AND BACKGROUND OF THE INVENTION

The present invention, in some embodiments thereof, relates to a T cell expressing an Fcγ receptor and methods of use thereof.

Cancer immunotherapy, including cell-based therapy, antibody therapy and cytokine therapy, has emerged in the last couple of years as a promising strategy for treating various types of cancer owing to its potential to evade genetic and cellular mechanisms of drug resistance and to target tumor cells while sparing healthy tissues.

Antibody-based cancer immunotherapies, such as monoclonal antibodies, antibody-fusion proteins, and antibody drug conjugates (ADCs) depend on recognition of cell surface molecules that are differentially expressed on cancer cells relative to non-cancerous cells and/or immune-checkpoint blockade. Binding of an antibody-based immunotherapy to a cancer cell can lead to cancer cell death via various mechanisms, e.g., antibody-dependent cell-mediated cytotoxicity (ADCC), complement-dependent cytotoxicity (CDC), direct cytotoxic activity of the payload from an antibody-drug conjugate (ADC) or suppressive checkpoint blockade. Many of these mechanisms initiate through the binding of the Fc domain of cell-bound antibodies to specialized cell surface receptors (Fc receptors) on hematopoietic cells.

Cell-based therapy using e.g. T cells having a T cell receptor (TCR) specific for an antigen differentially expressed in association with an MHC class I molecule on cancer cells relative to non-cancerous cells were shown to exert anti-tumor effects in several types of cancers, e.g. hematologic malignancies. However, antigen-specific effector lymphocytes, are very rare, individual-specific, limited in their recognition spectrum and difficult to obtain against most malignancies.

Strategies combining principles of antibody-based cancer immunotherapy and cell based therapy, such as CAR T cells and combined treatment with antibodies and T cells expressing Fc receptors have been disclosed (see e.g. EP Patent No: EP0340793; International Patent Application Publication No: WO2017205254; US Patent Application Publication Nos: US20150139943, US20180008638 and US20160355566; and Clemenceau et al. Blood. 2006; 107:4669-4677). However, attempts made to date to harness these cells against solid tumors were disappointing. Thus, an urgent need to develop treatments capable of eradicating solid tumors, which feature a higher safety profile and do not depend exclusively on the host T-cell repertoire, still remains.

Additional background art includes U.S. Pat. Nos. 8,313,943 and 6,111,166; and International Patent Application Publication No: WO2015121454.

### SUMMARY OF THE INVENTION

According to an aspect of some embodiments of the present invention there is provided a T cell genetically engineered to express a first polypeptide comprising an amino acid sequence of an Fc receptor common γ chain (FcRγ), the amino acid sequence is capable of transmitting an activating signal; and a second polypeptide comprising an extracellular ligand-binding domain of an Fcγ receptor capable of binding an Fc ligand and an amino acid sequence capable of recruiting the first polypeptide such that upon binding of the Fc ligand to the extracellular ligand-binding domain of the Fcγ receptor the activating signal is transmitted.

According to some embodiments of the invention, the first polypeptide comprises an amino acid sequence of a CD3ζ chain capable of transmitting an activating signal.

According to some embodiments of the invention, the FcRγ is located N-terminally to the CD3ζ chain.

According to some embodiments of the invention, the second polypeptide comprises an amino acid sequence of a CD3ζ chain capable of transmitting an activating signal.

According to some embodiments of the invention, the amino acid sequence capable of recruiting the first polypeptide comprises the transmembrane domain of an Fc receptor.

According to some embodiments of the invention, the amino acid sequence capable of recruiting the first polypeptide comprises the cytoplasmic domain of an Fc receptor.

According to some embodiments of the invention, the Fc receptor is Fcγ receptor.

According to some embodiments of the invention, the Fcγ receptor is CD64.

According to some embodiments of the invention, the first polypeptide is less than 25 kDa in molecular weight.

According to some embodiments of the invention, the first polypeptide does not comprise a target-binding moiety.

According to some embodiments of the invention, the first polypeptide does not comprise a scFv.

According to some embodiments of the invention, the second polypeptide does not comprise a scFv.

According to some embodiments of the invention, the T cell does not express a chimeric antigen receptor (CAR).

According to an aspect of some embodiments of the present invention there is provided a T cell clone expressing CD64, the CD64 comprises an extracellular domain, a transmembrane domain and a cytoplasmic domain.

According to an aspect of some embodiments of the present invention there is provided an isolated population of T cells comprising at least 80% T cells expressing endogenous CD64, the CD64 comprising an extracellular domain, a transmembrane domain and a cytoplasmic domain.

According to an aspect of some embodiments of the present invention there is provided a T cell genetically engineered to express CD64, the CD64 comprising an extracellular domain, a transmembrane domain and a cytoplasmic domain.

According to some embodiments of the invention, the T cell or the population of T cells, being genetically engineered to express a polypeptide comprising an amino acid sequence of an Fc receptor common γ chain (FcRγ), the amino acid sequence is capable of transmitting an activating signal.

According to some embodiments of the invention, the polypeptide further comprises an amino acid sequence of a CD3ζ chain, the amino acid sequence is capable of transmitting an activating signal.

According to some embodiments of the invention, the T cell or the population of T cells, being endogenously expressing a T cell receptor specific for a pathologic cell.

According to some embodiments of the invention, the T cell or the population of T cells, being genetically engineered to express a T cell receptor (TCR).

According to some embodiments of the invention, the T cell or the population of T cells, being genetically engineered to express a chimeric antigen receptor (CAR).

According to an aspect of some embodiments of the present invention there is provided a method of treating a disease associated with a pathologic cell in a subject treated with a therapeutic composition comprising an Fc domain, the therapeutic composition being specific for the pathologic cell, the method comprising administering to the subject a therapeutically effective amount of the T cells or the population of T cells, thereby treating the disease in the subject.

According to an aspect of some embodiments of the present invention there is provided the T cells or the population of T cells, for use in treating a disease associated with a pathologic cell in a subject treated with a therapeutic composition comprising an Fc domain, the therapeutic composition being specific for the pathologic cell.

According to an aspect of some embodiments of the present invention there is provided a method of treating a disease associated with a pathologic cell in a subject in need thereof, the method comprising administering to the subject a therapeutically effective amount of the T cells or the population of T cells; and a therapeutic composition comprising an Fc domain, the therapeutic composition being specific for the pathologic cell, thereby treating the disease in the subject.

According to an aspect of some embodiments of the present invention there is provided the T cells or the population of T cells; and a therapeutic composition comprising an Fc domain, for use in treating a disease associated with a pathologic cell in a subject in need thereof, wherein the therapeutic composition is specific for the pathologic cell.

According to an aspect of some embodiments of the present invention there is provided an article of manufacture comprising a packaging material packaging the T cells or the population of T cells and a therapeutic composition comprising an Fc domain.

According to some embodiments of the invention, the therapeutic composition is specific for a pathologic cell.

According to some embodiments of the invention, the therapeutic composition is an Fc-fusion protein.

According to some embodiments of the invention, the therapeutic composition is an antibody.

According to some embodiments of the invention, the antibody is an IgG.

According to some embodiments of the invention, the disease is cancer and wherein the pathologic cell is a cancerous cell.

According to some embodiments of the invention, the cancer is selected from the group consisting of melanoma, adenocarcinoma, mammary carcinoma, colon cancer, ovarian cancer, lung cancer and B-cell lymphoma.

According to some embodiments of the invention, the cancer is selected from the group consisting of melanoma, adenocarcinoma and mammary carcinoma.

According to some embodiments of the invention, the antibody is selected from the group consisting of Atezolizumab, Cetuximab, Retuximab, Gatipotuzumab and IVIG.

According to some embodiments of the invention, the cancerous cell expresses a marker selected from the group consisting of PDL-1, CD19, E-cadherin, MUC1, TRP-1 and TRP-2.

According to some embodiments of the invention, the cancerous cell expresses PDL-1.

According to some embodiments of the invention, the antibody is an anti-PDL-1.

According to some embodiments of the invention, the antibody is Atezolizumab.

According to an aspect of some embodiments of the present invention there is provided a method of isolating a T cell, the method comprising isolating a CD64+ T cell from a biological sample of a subject using an agent that binds CD64 polypeptide or a polynucleotide encoding the CD64 polypeptide.

According to some embodiments of the invention, the method comprising at least one of culturing, cloning, activating and genetically engineering the CD64+ T cell following the isolating.

According to some embodiments of the invention, the method comprising administering a plurality of the CD64+ T cell to a subject in need thereof.

According to some embodiments of the invention, the T cell is a CD4+ T cell.

According to some embodiments of the invention, the T cell is a CD8+ T cell.

According to some embodiments of the invention, the T cell is a proliferating cell.

According to some embodiments of the invention, the T cells are autologous to the subject.

Unless otherwise defined, all technical and/or scientific terms used herein have the same meaning as commonly understood by one of ordinary skill in the art to which the invention pertains. Although methods and materials similar or equivalent to those described herein can be used in the practice or testing of embodiments of the invention, exemplary methods and/or materials are described below. In case of conflict, the patent specification, including definitions, will control. In addition, the materials, methods, and examples are illustrative only and are not intended to be necessarily limiting.

### BRIEF DESCRIPTION OF THE SEVERAL VIEWS OF THE DRAWINGS

The patent or application file contains at least one drawing executed in color. Copies of this patent or patent application publication with color drawing(s) will be provided by the Office upon request and payment of the necessary fee.

Some embodiments of the invention are herein described, by way of example only, with reference to the accompanying drawings. With specific reference now to the drawings in detail, it is stressed that the particulars shown are by way of example and for purposes of illustrative discussion of embodiments of the invention. In this regard, the description taken with the drawings makes apparent to those skilled in the art how embodiments of the invention may be practiced.

In the drawings:

FIGS. 1A-1E demonstrate that adoptive transfer of CD4⁺ T cells along with tumor-binding antibodies induces direct killing of tumor cells. FIG. 1A is a schematic illustration of the experimental outline. FIG. 1B is a graph demonstrating B16F10 tumor size (mm²) in wild type (WT) mice following injection of CD4⁺ T cells obtained from the peripheral blood (PB), tumor draining lymph node (DLN) or tumor in combination with antibodies against the melanoma antigen TRP1 (n=4), as compared to untreated control WT mice. FIG. 1C shows photomicrographs of B16F10 tumor-bearing WT mice, 14 days following injection of CD4⁺ T cells obtained from peripheral blood (PB), tumor draining lymph node (DLN) or tumor in combination with anti-TRP1 antibodies, as compared to untreated control mice (PBS). FIG. 1D is a graph demonstrating B16F10 tumor size (mm²) in RAG deficient mice following adoptive transfer of CD4⁺ T cells with or without antibodies against TRP (n=4). FIG. 1E is a graph demonstrating B16F10 tumor size (mm²) following adoptive transfer of the indicated CD4+T clones with or without antibodies against TRP1 and Ovalbumin (n=4). Each graph summarizes the results of a representative experiment out of at least 3 performed.

FIGS. 2A-2C demonstrate that a subset of CD4⁺ T cells in tumor-bearing mice expresses Fcγ receptors. FIG. 2A shows representative flow cytometry analysis of the indicated Fcγ receptors expression on CD4⁺ T cells from tumor-bearing mice (n=4). FIG. 2B is a graph demonstrating the percentages of CD4⁺ T cells from B16F10 tumor-bearings mice that express the indicated Fcγ receptors (n=5). FIG. 2C is a graph demonstrating the percentages of CD4⁺ T cells from 4T1 tumor-bearing mice that express the indicated Fcγ receptors (n=4). Each graph summarizes the results of a representative experiment out of 3 performed.

FIGS. 3A-3E demonstrate that a subset of CD4⁺ T cells in lymphoid organs of naïve mice expresses Fcγ receptors. FIG. 3A is a graph demonstrating the percentages CD4⁺ T cells from naïve mice that express the indicated Fcγ receptors in various organs. FIG. 3B shows representative FACS sort of splenic CD4⁺ T cells that express FcγRI (CD64) (n=5). FIG. 3C shows confocal microscopy images of splenic CD4⁺ T cells sorted by expression of FcγRI and stained with the indicated markers (n=3, magnification=×600). FIG. 3D shows mRNA transcription levels of CD3 (left panel) and FcγRI (right panel) in splenic CD4⁺ T cells sorted by expression of FcγRI. Expression patterns are compared to sort splenic CD11b⁺ cells. NGC=negative control. FIG. 3E shows confocal microscopy images of histological sections of naïve mouse spleen stained with the indicated markers.

FIGS. 4A-4D demonstrate that expression of FcγRI and its signaling chain in tumor specific CD4⁺ T cells induces effective lysis of tumor cells coated with antibodies. FIG. 4A shows representative confocal microscopy images of splenic FcγRI⁺/CD4⁺ T cells isolated from wild type mice (WT CD4) or OT-II mice (OT-II CD4) and incubated overnight with GFP-labeled B16 cells with or without the indicated antibodies (magnification=×800, n=5). FIG. 4B is a graph demonstrating Biotek H1M fluorescence reads of GFP-labeled B16 cells cultured overnight with splenic FcγRI⁺/CD4⁺ (FcRI) or FcγRI^{neg}/CD4⁺[FcRI(neg)]T cells isolated from WT mice, with or without the indicated antibodies. The graph shows results pooled from 3 experiments. FIG. 4C shows representative fluorescence microscopy images of splenic CD4⁺ T cells infected with anti-tumor TCR, FcγRI and FcRγ and incubated overnight with GFP-labeled B16 cells with or without the indicated antibodies (magnification=×400, n=2). FIG. 4D shows the in-vivo anti-tumor effect of splenic CD4⁺ T cells infected with anti-tumor TCR, FcγRI and FcRγ in an adoptive transfer model, with or without an anti-TRP1 antibody.

FIGS. 5A-5C demonstrate that expression of FcγRI and its signaling chain in naïve C57BL WT CD4⁺ or CD8⁺ T cells induces effective lysis of tumor cells coated with antibodies. FIG. 5A shows schematic illustrations of the constructs used: a construct encoding FcγRI T2A FcRγ (SEQ ID NOs: 21-22), a construct encoding FcγRI T2A FcRγ-CD3zeta (SEQ ID NOs: 23-24), a construct encoding FcγRI-CD3zeta T2A FcRγ (SEQ ID NOs: 27-28, see FIG. 6A), a construct encoding FcγRI-CD3zeta (SEQ ID NOs: 25-26) and a construct encoding and FcγRIα extracellular domain-TCRβconstant region (SEQ ID NO: 41-42). FIG. 5B shows images of B16 target cells co-cultured with CD4⁺ and CD8⁺ T cells infected with the constructs shown in FIG. 5A. The cells where co-cultured in 96 wells plate, with or without an anti-TRP1 antibody for 48 hours; and images where taken under ×100 magnitude in inverted light microscope. FIG. 5C is a graph demonstrating flow cytometry analysis of annexin-V/PI staining for apoptotic B16 cells co-cultured with anti-TRP1 antibody and CD8⁺ T cells transduced with the different constructs described above.

FIGS. 6A-6C are schematic illustrations of the designed constructs and the resultant receptors expressed. FIG. 6A shows schemes of two optional constructs: in the first (left panel) the CD3ζ (zeta) chain is connected to the FcγRIα chain and the Fc receptor γ chain (FcRγ) is separated by T2A; in the second (right panel), the CD3ζ (zeta) chain is connected to the FcRγ signaling chain and both are separated by T2A from FcγRIα chain, FIG. 6B shows illustrations of the transduced cells and the receptors which are expressed: in the left panel, the CD3ζ chain is fused to FcγRIα; and in the right panel, the FcγRIα is expressed in parallel to the FcRγ-CD3ζ fusion protein. FIG. 6C shows a scheme of the therapeutic procedure. Namely, T cells are isolated from peripheral blood of a tumor-bearing patient and infected with e.g. one of the constructs shown in FIGS. 6A-6B. Following, several millions of transduced T cells are infused back to the patient along with clinically-approved tumor-binding antibodies.

FIG. 7 shows schematic illustrations of a construct encoding FcγRIα and FcRγ as a single polypeptide (SEQ ID NOs: 29-30) and a construct encoding FcγRIα, FcRγ and CD3zeta as a single polypeptide (SEQ ID NOs: 31-32).

FIG. 8 shows confocal microscopy images of cells expressing the FcγRIα-2A-FcRγ construct and stained for TCRβ, FcγRI and GFP. ×200 magnitude.

FIG. 9 is a graph demonstrating the correlation between the number of cells counted by incuCyte imager in a field and the number of B16-H2B-tdTomato cells cultured in a well of 96 wells plate.

FIGS. 10A-10B demonstrate killing of B16 target cells by FcγRIα-2A-FcRγ infected cells in different ratios. FIG. 10A shows representative images taken by incuCyte imager following 2 days of co-culturing CD8+ T cells infected with FcγRIα-2A-FcRγ and B16-H2B-tdTomato in different effector:target ratios ranging from 0.5:1 to 16:1, in the presence of an anti-TRP-1 antibody. ×100 magnitude. FIG. 10B is a graph demonstrating the number of target cells counted by the incuCyte imager, following 2 days of co-culturing CD4+ or CD8+ T cells infected with FcγRIα-2A-FcRγ and B16-H2B-tdTomato in different effector:target ratios with or without an anti-TRP-1 antibody.

FIGS. 11A-11C demonstrate the superiority of expressing two distinct polypeptides, one comprising the ligand binding domain of FcγRIα and the other comprising FcRγ, as compared to a single polypeptide expressing both. FIG. 11A shows schematic illustrations of a construct encoding a single polypeptide comprising FcγRIα extracellular domain-CD8a hinge and transmembrane domain-FcRγ intracellular domain (SEQ ID NOs: 43-44). FIG. 11B shows representative images of B16-H2B-tdTomato target cells treated with anti-TRP-1 antibody either alone or in combination with co-culturing with uninfected CD8+ T cells (Sham) or CD8+ T cells infected with the FcγRIα extracellular domain-CD8a hinge and transmembrane domain-FcRγ intracellular domain construct. Images were taken with bright light, red and green filters, ×100 magnitude, following 48 hours of co-culture. FIG. 11C is a graph demonstrating the number of target cells counted by the incuCyte imager, following 48 hours of co-culturing as described in FIG. 11B hereinabove.

### DESCRIPTION OF SPECIFIC EMBODIMENTS OF THE INVENTION

The present invention, in some embodiments thereof, relates to a T cell expressing an Fcγ receptor and methods of use thereof.

Before explaining at least one embodiment of the invention in detail, it is to be understood that the invention is not necessarily limited in its application to the details set forth in the following description or exemplified by the Examples. The invention is capable of other embodiments or of being practiced or carried out in various ways.

Antibody-based cancer immunotherapies depend on recognition of cell surface molecules that are differentially expressed on cancer cells relative to non-cancerous cells and/or immune-checkpoint blockade. Binding of an antibody-based immunotherapy to a cancer cell can lead to cancer cell death via various mechanisms, many of them initiate through the binding of the Fc domain of cell-bound antibodies to specialized cell surface receptors (Fc receptors) on hematopoietic cells.

On the other hand, cell-based therapy using e.g. T cells having a T cell receptor (TCR) specific for an antigen differentially expressed in association with an MHC class I molecule on cancer cells relative to non-cancerous cells were shown to exert anti-tumor effects in several types of cancers, e.g. hematologic malignancies.

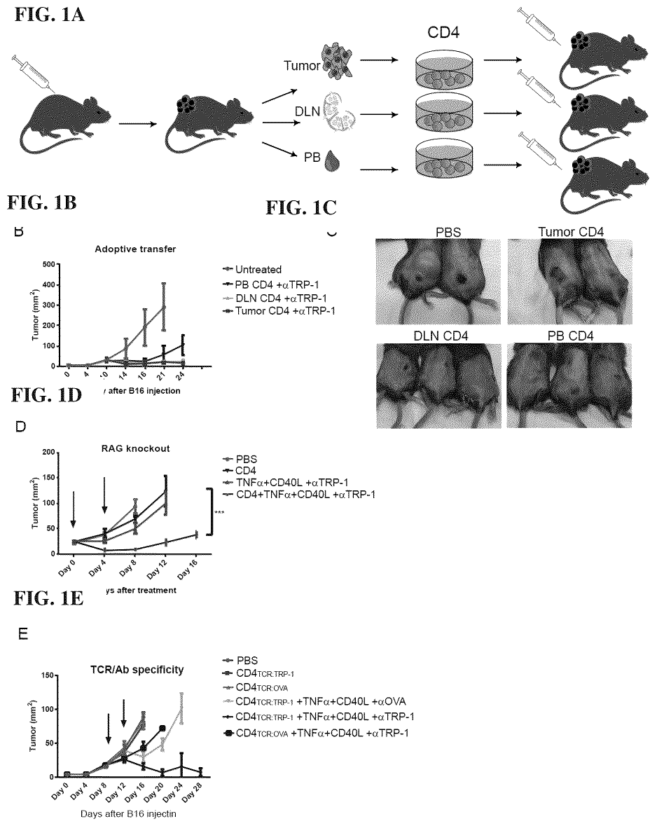

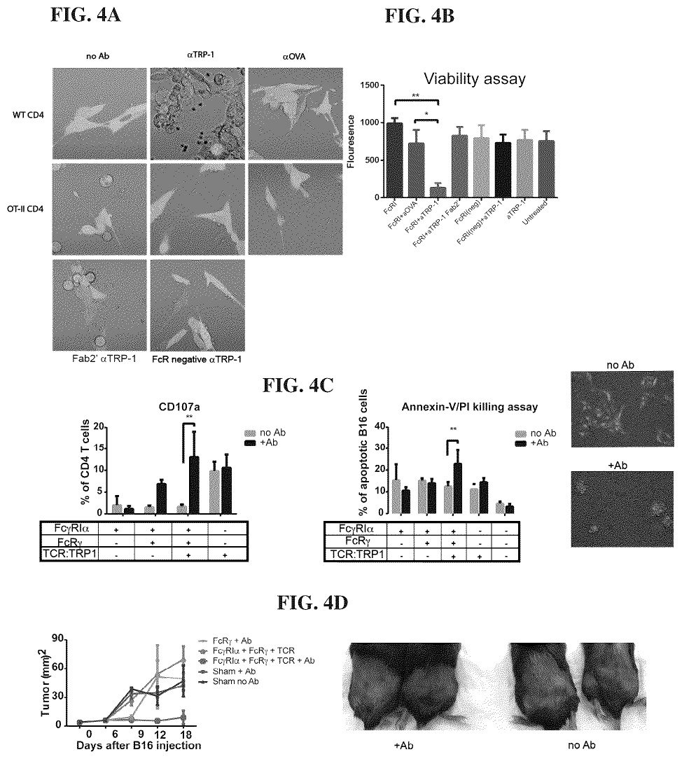

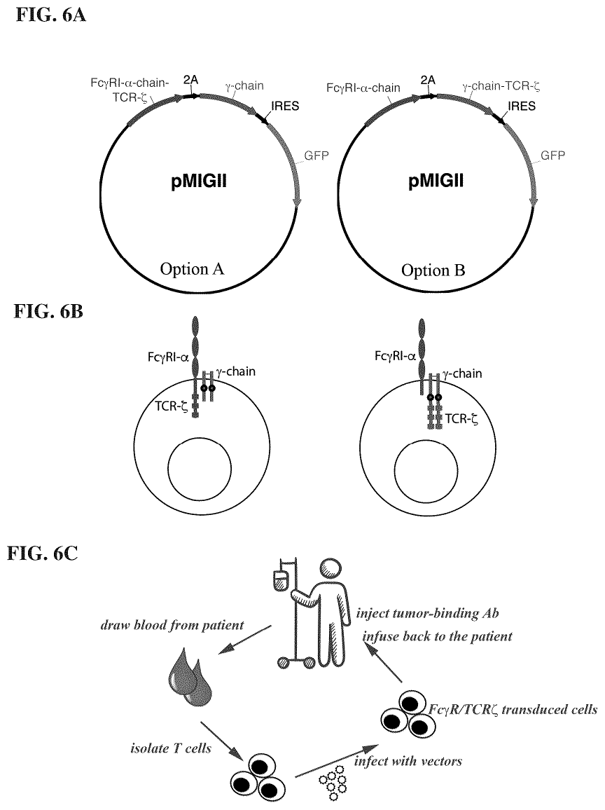

Whilst reducing the present invention to practice, the present inventors have now discovered a novel subset of CD4⁺ T which express the high affinity Fcγ receptor FcγRI (CD64). Such tumor specific FcγRI⁺CD4⁺ T cells were able to bind tumor cells coated with anti-tumor antibodies and secrete lytic granules resulting in a remarkable tumor lysis (Example 1, FIGS. 1A-4B). Following, the present inventors were able to recapitulate the cytotoxic capacities of this unique CD4⁺ T cell population in conventional CD4⁺ and CD8⁺ T cells by exogenously expressing an FcγRI polypeptide and an Fcγ chain polypeptide (Examples 2-3, FIGS. 4C-11C). Indeed, these engineered T cells exerted remarkable killing capabilities of tumors in combination with anti-tumor antibodies.

Consequently, specific embodiments of the present teachings suggest T cells genetically engineered to express two distinct polypeptides, one comprising a ligand binding domain of an Fcγ receptor and the other comprising an Fc receptor common 7 chain; and methods of using these T cells to treat diseases associated with pathologic cells (e.g. cancer).

Thus, according to a first aspect of the present invention, there is provided a T cell genetically engineered to express a first polypeptide comprising an amino acid sequence of an Fc receptor common 7 chain (FcRγ), said amino acid sequence is capable of transmitting an activating signal; and a second polypeptide comprising an extracellular ligand-binding domain of an Fcγ receptor capable of binding an Fc ligand and an amino acid sequence capable of recruiting said first polypeptide such that upon binding of said Fc ligand to said extracellular ligand-binding domain of said Fcγ receptor said activating signal is transmitted.

As used herein, the term “T cell” refers to a differentiated lymphocyte with a CD3+, T cell receptor (TCR)+ having either CD4+ or CD8+ phenotype.

According to specific embodiments, the T cell is an effector cell.

As used herein, the term “effector T cell” refers to a T cell that activates or directs other immune cells e.g. by producing cytokines or has a cytotoxic activity e.g., CD4+, Th1/Th2, CD8+ cytotoxic T lymphocyte.

According to specific embodiments, the T cell is a CD4+ T cell.

According to other specific embodiments, the T cell is a CD8+ T cell.

According to specific embodiments, the T cell is a naïve T cell.

According to specific embodiments, the T cell is a memory T cell. Non-limiting examples of memory T cells include effector memory CD4+ T cells with a CD3+/CD4+/CD45RA−/CCR7− phenotype, central memory CD4+ T cells with a CD3+/CD4+/CD45RA−/CCR7+ phenotype, effector memory CD8+ T cells with a CD3+/CD8+CD45RA−/CCR7− phenotype and central memory CD8+ T cells with a CD3+/CD8+CD45RA−/CCR7+ phenotype.

According to specific embodiments, the T cell is a proliferating cell.

As used herein, the phrase“proliferating cell” refers to a T cell that proliferated upon stimulation as defined by a cell proliferation assay, such as, but not limited to, CFSE staining, MTS, Alamar blue, BRDU, thymidine incorporation, and the like.

According to specific embodiments, the T cell is a proliferating CD4+ T cell.

According to specific embodiments, the T cell is a proliferating CD8+ T cell.

According to specific embodiments, the T cell is a human T cell.

Methods of obtaining T cells from a subject are well known in the art, such as drawing whole blood from a subject and collection in a container containing an anti-coagulant (e.g. heparin or citrate); and apheresis followed by a purification process. There are several methods and reagents known to those skilled in the art for purifying T cells from whole blood such as leukapheresis, sedimentation, density gradient centrifugation (e.g. ficoll), centrifugal elutriation, fractionation, chemical lysis of e.g. red blood cells (e.g. by ACK), selection using cell surface markers (using e.g. FACS sorter or magnetic cell separation techniques such as are commercially available e.g. from Invitrogen, Stemcell Technologies, Cellpro, Advanced Magnetics, or Miltenyi Biotec.), and depletion of specific non-T cells cell types by methods such as eradication (e.g. killing) with specific antibodies or by affinity based purification based on negative selection (using e.g. magnetic cell separation techniques, FACS sorter and/or capture ELISA labeling). Such methods are described for example in THE HANDBOOK OF EXPERIMENTAL IMMUNOLOGY, Volumes 1 to 4, (D. N. Weir, editor) and FLOW CYTOMETRY AND CELL SORTING (A. Radbruch, editor, Springer Verlag, 2000).

According to specific embodiments, the T cell is obtained from a healthy subject.

According to specific embodiments, the T cell is obtained from a subject suffering from a pathology (e.g. cancer).

According to specific embodiments, the T cell is expressing a T cell receptor specific for a pathologic (diseased, e.g. cancerous) cell, i.e. recognizes an antigen presented in the context of MHC which is overexpressed or solely expressed by a pathologic cell as compared to a non-pathologic cell.

According to specific embodiments, the antigen is a cancer antigen, i.e. an antigen overexpressed or solely expressed by a cancerous cell as compared to a non-cancerous cell. A cancer antigen may be a known cancer antigen or a new specific antigen that develops in a cancer cell (i.e. neoantigens).

Non-limiting examples for known cancer antigens include MAGE-AI, MAGE-A2, MAGE-A3, MAGE-A4, MAGE-AS, MAGE-A6, MAGE-A7, MAGE-AS, MAGE-A9, MAGE-AIO, MAGE-All, MAGE-A12, GAGE-I, GAGE-2, GAGE-3, GAGE-4, GAGE-5, GAGE-6, GAGE-7, GAGE-8, BAGE-1, RAGE-1, LB33/MUM-1, PRAME, NAG, MAGE-Xp2 (MAGE-B2), MAGE-Xp3 (MAGE-B3), MAGE-Xp4 (MAGE-B4), MAGE-Cl/CT7, MAGE-C2, NY-ESO-1, LAGE-1, SSX-1, SSX-2(HOM-MEL-40), SSX-3, SSX-4, SSX-5, SCP-1 and XAGE, melanocyte differentiation antigens, p53, ras, CEA, MUCI, PMSA, PSA, tyrosinase, Melan-A, MART-I, gplOO, gp75, alphaactinin-4, Bcr-Abl fusion protein, Casp-8, beta-catenin, cdc27, cdk4, cdkn2a, coa-1, dek-can fusion protein, EF2, ETV6-AML1 fusion protein, LDLR-fucosyltransferaseAS fusion protein, HLA-A2, HLA-All, hsp70-2, KIAA0205, Mart2, Mum-2, and 3, neo-PAP, myosin class I, OS-9, pml-RAR alpha fusion protein, PTPRK, K-ras, N-ras, Triosephosphate isomerase, GnTV, Herv-K-mel, NA-88, SP17, and TRP2-Int2, (MART-I), E2A-PRL, H4-RET, IGH-IGK, MYL-RAR, Epstein Barr virus antigens, EBNA, human papillomavirus (HPV) antigens E6 and E7, TSP-180, MAGE-4, MAGE-5, MAGE-6, p185erbB2, plSOerbB-3, c-met, nm-23H1, PSA, TAG-72-4, CA 19-9, CA 72-4, CAM 17.1, NuMa, K-ras, alpha.-fetoprotein, 13HCG, BCA225, BTAA, CA 125, CA 15-3 (CA 27.29\BCAA), CA 195, CA 242, CA-50, CAM43, CD68\KP1, CO-029, FGF-5, 0250, Ga733 (EpCAM), HTgp-175, M344, MA-50, MG7-Ag, MOV18, NB\70K, NYCO-I, RCASI, SDCCAG16, TA-90 (Mac-2 binding protein\cyclophilin C-associated protein), TAAL6, TAG72, TLP, TPS, tyrosinase related proteins, TRP-1, or TRP-2.

According to specific embodiments, the T cell is endogenously expressing a T cell receptor specific for a pathologic cell (e.g. cancerous cell).

According to specific embodiments, the T cell is an engineered T cells transduced with a T cell receptor (TCR).

As used herein the phrase “transduced with a TCR” or “genetically engineered to express a TCR” refers to cloning of variable α- and β-chains from T cells with specificity against a desired antigen presented in the context of MHC. Methods of transducing with a TCR are known in the art and are disclosed e.g. in Nicholson et al. Adv Hematol. 2012; 2012:404081; Wang and Riviere Cancer Gene Ther. 2015 March; 22(2):85-94); and Lamers et al, Cancer Gene Therapy (2002) 9, 613-623. According to specific embodiments, the TCR is specific for a pathologic cell.

According to specific embodiments, the T cell is an engineered T cells transduced with a chimeric antigen receptor (CAR).

As used herein, the phrase “transduced with a CAR” or “genetically engineered to express a CAR” refers to cloning of a nucleic acid sequence encoding a chimeric antigen receptor (CAR), wherein the CAR comprises an antigen recognition moiety and a T-cell activation moiety. A chimeric antigen receptor (CAR) is an artificially constructed hybrid protein or polypeptide containing an antigen binding domain of an antibody (e.g., a single chain variable fragment (scFv)) linked to T-cell signaling or T-cell activation domains. Method of transducing with a CAR are known in the art and are disclosed e.g. in Davila et al. Oncoimmunology. 2012 Dec. 1; 1(9):1577-1583; Wang and Riviere Cancer Gene Ther. 2015 March; 22(2):85-94); Maus et al. Blood. 2014 Apr. 24; 123(17):2625-35; Porter D L The New England journal of medicine. 2011, 365(8):725-733; Jackson H J, Nat Rev Clin Oncol. 2016; 13(6):370-383; and Globerson-Levin et al. Mol Ther. 2014; 22(5):1029-1038. According to specific embodiments, the antigen recognition moiety is specific for a pathologic cell.

According to other specific embodiments, the T cell is not transduced (i.e. does not express) a CAR.

The T cell of some embodiments of the invention is genetically engineered to express a first polypeptide comprising an amino acid sequence of an Fc receptor common 7 chain (FcRγ) which is capable of transmitting an activating signal.

As used herein the phrase “Fc receptor common 7 chain” abbreviated as “FcRγ” refers to the polypeptide expression product of the FCER1G gene (Gene ID 2207). According to specific embodiments, FcRγ is human FcRγ. According to a specific embodiment, the FcRγ protein refers to the human protein, such as provided in the following GenBank Number NP_004097.

According to specific embodiments, the polypeptide of some embodiments of the invention comprises a full length FcRγ polypeptide.

According to specific embodiments, the polypeptide of some embodiments of the invention comprises a functional fragment of FcRγ polypeptide.

As used herein, the phrase “functional fragment of FcRγ polypeptide”, refers to a portion of the polypeptide which comprises a transmembrane domain and an intracellular domain and maintains at least the capability of transmitting an activating signal in a cell expressing an Fcγ receptor upon binding of the Fcγ receptor to a Fc ligand.

According to specific embodiments, the functional fragment of FcRγ polypeptide is capable of forming a homodimer.

According to specific embodiments, the functional fragment of the FcRγ polypeptide comprises an ITAM motif.

As used herein the terms “activating” or “activation” refer to the process of stimulating a T cell that results in cellular proliferation, maturation, cytokine production and/or induction of effector functions.

Methods of determining signaling of an activating signal are well known in the art, and include, but are not limited to, enzymatic activity assays such as kinase activity assays, and expression of molecules involved in the signaling cascade using e.g. PCR, Western blot, immunoprecipitation and immunohistochemistry. Additionally or alternatively, determining transmission of an activating signal can be effected by evaluating T cell activation or function. Methods of evaluating T cell activation or function are well known in the art and include, but are not limited to, proliferation assays such as CFSE staining, MTT, Alamar blue, BRDU and thymidine incorporation, cytotoxicity assays such as CFSE staining, chromium release, Calcin AM, cytokine secretion assays such as intracellular cytokine staining, ELISPOT and ELISA, expression of activation markers such as CD25, CD69, CD137, CD107a, PD1, and CD62L using flow cytometry.

According to specific embodiments, the polypeptide of some embodiments of the invention comprises an FcRγ polypeptide amino acid sequence comprising SEQ ID NO: 13.

According to specific embodiments, the polypeptide of some embodiments of the invention comprises an FcRγ polypeptide amino acid sequence consisting of SEQ ID NO: 13.

According to specific embodiments, the polypeptide of some embodiments of the invention comprises an amino acid sequence as set forth in SEQ ID NO: 15.

According to specific embodiments, the polypeptide of some embodiments of the invention comprises an FcRγ polypeptide amino acid sequence consisting of SEQ ID NO: 15.

The term “FcRγ” also encompasses functional homologues (naturally occurring or synthetically/recombinantly produced), which exhibit the desired activity as defined hereinabove (i.e., capability of transmitting an activating signal, forming a homodimer). Such homologues can be, for example, at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% or 100% identical or homologous to the polypeptide SEQ ID Nos: 13 or 15; or at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% or 100% identical to the polynucleotide sequence encoding same e.g. SEQ ID Nos: 14 or 16.

Sequence identity or homology can be determined using any protein or nucleic acid sequence alignment algorithm such as Blast, ClustalW, and MUSCLE.

The homolog may also refer to an ortholog, a deletion, insertion, or substitution variant, including an amino acid substitution, as further described hereinbelow.

According to specific embodiments, the FcRγ polypeptide may comprise conservative and non-conservative amino acid substitutions.

According to specific embodiments, the polypeptide comprising an amino acid sequence of an FcRγ, is less than 100 kDa, less than 50 kDa, less than 25 kDa or less than 20 kDa in molecular weight, each possibility represents a separate embodiment of the present invention.

According to specific embodiments, the polypeptide comprising an amino acid sequence of an FcRγ is less than 25 kDa in molecular weight.

According to specific embodiments, the polypeptide comprising an amino acid sequence of an FcRγ does not comprise a target-binding moiety.

As used herein, the “target binding moiety” is an antigen binding moiety such as an antibody e.g. single chain antibody (e.g., scFv).

Thus, according to specific embodiments, the polypeptide comprising an amino acid sequence of an FcRγ does not comprise a scFv.

The T cell of some embodiments of the invention is genetically engineered to express a second polypeptide comprising an extracellular ligand-binding domain of an Fcγ receptor capable of binding an Fc ligand and an amino acid sequence capable of recruiting the first polypeptide (which comprises an amino acid sequence of an FcRγ).

As used herein the phrase “extracellular ligand-binding domain of Fcγ receptor” refers to at least a fragment of an Fcγ receptor which comprises an extracellular domain capable of binding an Fc ligand.

As used herein, the term “Fc ligand” refers to an Fc domain such as of an antibody. According to specific embodiments, the Fc ligand is an IgG Fc domain.

Assays for testing binding are well known in the art and include, but not limited to flow cytometry, BiaCore, bio-layer interferometry Blitz® assay, HPLC, surface plasmon resonance.

According to specific embodiments, the extracellular ligand-binding domain of Fcγ receptor binds the Fc ligand with a Kd>10⁻⁶ M, >10⁻⁷ M, >10⁻⁸ M or >10⁻⁹ M, each possibility represents a separate embodiment of the present invention.

According to specific embodiments, the extracellular ligand-binding domain of Fc receptor binds the Fc ligand with a Kd>10⁻⁹ M.

As used herein, the term “Fcγ receptor” refers to a cell surface receptor which exhibits binding specificity to the Fc domain of an IgG antibody. Examples of Fcγ receptors include, without limitation, CD64A, CD64B, CD64C, CD32A, CD32B, CD16A, and CD16B. The term “Fcγ receptor” also encompasses functional homologues (naturally occurring or synthetically/recombinantly produced) and/or Fc receptors comprising conservative and non-conservative amino acid substitutions, which exhibit the desired activity (i.e., capability of binding an IgG Fc binding domain).

According to specific embodiments, the Fcγ receptor is CD64.

As used herein, the term “CD64”, also known as FcγRI, refers to the polypeptide expression product of the FCGR1A, FCGR1B or FCGR1C gene (Gene ID 2209, 2210, 2211, respectively), and includes CD64A, CD64B and CD64C. Full length CD64 comprises an extracellular, transmembrane and an intracellular domain and is capable of at least binding an IgG (IgG1 and IgG3) Fc domain and recruiting an FcRγ. Methods of determining binding and recruitment of an FcRT are well known in the art and are also described hereinabove and below.

According to specific embodiments, CD64 is human CD64. According to a specific embodiment, the CD64 protein refers to the human CD64A protein, such as provided in the following UniProt Number P12314.

According to a specific embodiment, the CD64 protein refers to the human CD64B protein, such as provided in the following UniProt Number Q92637.

According to a specific embodiment, the CD64 protein refers to the human CD64C protein, such as provided in the following GenBank Number XM_001133198.

According to specific embodiments, CD64 amino acid sequence comprises SEQ ID NO: 5.

According to specific embodiments, CD64 comprises a functional fragment of a CD64 polypeptide.

As use herein, the phrase “functional fragment of a CD64 polypeptide”, refers to a portion of the polypeptide which maintains at least the capability of binding an IgG (IgG1 and IgG3) Fc domain and/or recruiting an FcRγ, as further described hereinbelow.

The term “CD64” also encompasses functional homologues (naturally occurring or synthetically/recombinantly produced), which exhibit the desired activity (i.e., binding an IgG (IgG1 and IgG3) Fc domain and/or recruiting an FcRγ). Such homologues can be, for example, at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% or 100% identical or homologous to the polypeptide SEQ ID No: 5; or at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% or 100% identical to the polynucleotide sequence encoding same e.g. SEQ ID NO: 6.

According to specific embodiments, the CD64 polypeptide may comprise conservative and non-conservative amino acid substitutions.

Thus, the polypeptide of some embodiments of the invention comprises an extracellular ligand-binding domain of CD64.

According to specific embodiments, the polypeptide comprises an extracellular ligand-binding domain of CD64 comprising SEQ ID NO: 7.

According to specific embodiments, the polypeptide comprises an extracellular ligand-binding domain of CD64 consisting of SEQ ID NO: 7.

The second polypeptide of some embodiments of the invention comprises an amino acid sequence capable of recruiting the first polypeptide (i.e. which comprises an amino acid sequence of an FcRγ), such that upon binding of an Fc ligand to the extracellular ligand-binding domain of the Fcγ receptor an activating signal is transmitted by the first polypeptide.

According to specific embodiments, the amino acid sequence capable of recruiting the first polypeptide directly recruits the first polypeptide (i.e. without an intermediate polypeptide).

Such amino acid sequences are well known to the skilled in the art and include for example the transmembrane and/or the cytoplasmic domains of several Fc receptors such as, but not limited to CD64, CD16A, CD16B, FcRIβ, FcαRI (CD89).

According to specific embodiments, the amino acid sequence capable of recruiting the first polypeptide is not of an Fcε receptor (FcεR).

Methods of determining recruitment of the first polypeptide are well known in the art, and include, but are not limited to, enzymatic activity assays such as kinase activity assays, and expression of molecules involved in the signaling cascade using e.g. PCR, Western blot, immunoprecipitation and immunohistochemistry. Additionally or alternatively, determining recruitment of the first polypeptide can be effected by evaluating cell activation or function by methods well known in the art such as, but not limited to proliferation assays such as CFSE staining, MTT, Alamar blue, BRDU and thymidine incorporation, cytotoxicity assays such as CFSE staining, chromium release, Calcin AM, and the like. Exemplary methods for determining recruitment of an FcRγ are disclosed in e.g. in Kim, M. K., et al. (2003) Blood 101(11): 4479-4484; and Harrison, P. T., et al. (1995) Mol Membr Biol 12(4): 309-312, the contents of which are fully incorporated herein by reference.

According to specific embodiments, the amino acid sequence capable of recruiting the first polypeptide comprises the transmembrane domain of an Fc receptor.

According to specific embodiments, the amino acid sequence capable of recruiting the first polypeptide comprises the cytoplasmic domain of an Fc receptor.

According to specific embodiments, the amino acid sequence capable of recruiting the first polypeptide comprises the transmembrane domain of an Fcγ receptor.

According to specific embodiments, the amino acid sequence capable of recruiting the first polypeptide comprises the cytoplasmic domain of an Fcγ receptor.

According to specific embodiments, the amino acid sequence capable of recruiting the first polypeptide comprises an amino acid sequence of CD64 capable of recruiting said first polypeptide.

Thus, according to specific embodiments, the amino acid sequence capable of recruiting the first polypeptide comprises the transmembrane domain of CD64.

According to specific embodiments, the amino acid sequence capable of recruiting the first polypeptide consists of the transmembrane domain of CD64.

According to specific embodiments, the amino acid sequence capable of recruiting the first polypeptide comprises SEQ ID NO: 9.

According to specific embodiments, the amino acid sequence capable of recruiting the first polypeptide consists of SEQ ID NO: 9.

According to specific embodiments, the amino acid sequence capable of recruiting the first polypeptide comprises the intracellular domain of CD64.

According to specific embodiments, the amino acid sequence capable of recruiting the first polypeptide comprises SEQ ID NO: 11.

According to specific embodiments, the amino acid sequence capable of recruiting the first polypeptide comprises the transmembrane domain and the intracellular domain of CD64.

According to specific embodiments, the amino acid sequence capable of recruiting the first polypeptide comprises SEQ ID NO: 33.

According to specific embodiments, the amino acid sequence capable of recruiting the first polypeptide consists of SEQ ID NO: 33.

According to specific embodiments, both the extracellular ligand-binding domain and the amino acid sequence capable of recruiting the first polypeptide, in the second polypeptide are of CD64.

Hence, according to specific embodiments, the second polypeptide comprises the extracellular domain and the transmembrane domain of CD64.

According to specific embodiments, the second polypeptide comprises SEQ ID NO: 34.

According to specific embodiments, the second polypeptide comprises an extracellular domain, a transmembrane domain and a cytoplasmic domain of CD64.

According to specific embodiments, the second polypeptide comprises SEQ ID NO: 5.

According to specific embodiments, the second polypeptide comprises an amino acid sequence of an extracellular ligand-binding domain of CD64 and an amino acid sequence capable of recruiting the first polypeptide consisting of SEQ ID NO: 5.

According to specific embodiments, the second polypeptide consists of SEQ ID NO: 5.

According to specific embodiments, the second polypeptide does not comprise an antibody.

According to a specific embodiment, the second polypeptide does not comprise a scFv.

According to specific embodiments, any of the first and second polypeptides comprises an amino acid sequence of a CD3ζ chain capable of transmitting an activating signal.

According to specific embodiments, the first polypeptide comprises an amino acid sequence of a CD3ζ chain.

According to specific embodiments, the FcRγ is located N-terminally to the CD3ζ chain.

According to specific embodiments, the second polypeptide comprises an amino acid sequence of a CD3ζ chain.

According to specific embodiments, the CD3ζ chain is located C-terminally to the amino acid sequence capable of recruiting the first polypeptide.

As used herein the term “CD3ζ chain” also known as TCRζ or CD247 refers to the polypeptide expression product of the CD247 gene (Gene ID 919). According to specific embodiments, CD3ζ chain is human CD3ζ chain. According to a specific embodiment, the CD3ζ chain protein refers to the human protein, such as provided in the following GenBank Numbers NP_000725 and/or NP_932170.

According to specific embodiments, the polypeptide of some embodiments of the invention comprises a full length CD3ζ chain polypeptide.

According to specific embodiments, the polypeptide of some embodiments of the invention comprises a functional fragment of CD3 chain polypeptide.

As use herein, the phrase “functional fragment of a CD3z”, refers to a portion of the polypeptide which comprises at least an intracellular domain and maintains at least the capability of transmitting an activating signal in a T cell. Typically, such an amino acid sequence comprises an ITAM motif.

According to specific embodiments, the amino acid sequence of a CD3ζ chain comprises SEQ ID NO: 17.

According to specific embodiments, the amino acid sequence of a CD3ζ chain consists of SEQ ID NO: 17.

According to specific embodiments, the amino acid sequence of a CD3ζ chain comprises SEQ ID NO: 19.

According to specific embodiments, the amino acid sequence of a CD3ζ chain consists of SEQ ID NO: 19.

The term “CD3ζ chain” also encompasses functional homologues (naturally occurring or synthetically/recombinantly produced), which exhibit the desired activity (i.e., capability of transmitting an activating signal). Such homologues can be, for example, at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% or 100% identical or homologous to the polypeptide SEQ ID Nos: 17 or 19; or at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% or 100% identical to the polynucleotide sequence encoding same.

According to specific embodiments, the CD3ζ chain polypeptide may comprise conservative and non-conservative amino acid substitutions.

According to specific embodiments, any of the polypeptides disclosed herein can comprise a co-stimulatory signaling domain.

According to other specific embodiments, the polypeptides disclosed herein do not comprise a co-stimulatory signaling domain.

According to specific embodiments, any of the first and second polypeptides comprises a co-stimulatory signaling domain.

According to specific embodiments, the first polypeptide does not comprise a co-stimulatory signaling domain.

According to specific embodiments, the second polypeptide does not comprise a co-stimulatory signaling domain.

As used herein, the phrase “co-stimulatory signaling domain” refers to an amino acid sequence of a co-stimulatory molecule capable of transmitting a secondary stimulatory signal resulting in activation of the T cell. Typically, a co-stimulatory signaling domain does not comprise an ITAM domain.

Any known co-stimulatory signaling domain can be used with specific embodiments of the present invention. Non-limiting examples of co-stimulatory signaling domains include 4-1BB, CD28, OX40, ICOS, CD27, GITR, HVEM, TIM1, LFA1(CD11a), CD2.

According to specific embodiments, the co-stimulatory signaling domain is of 4-1BB and/or OX40.

Non-limiting examples of specific sequences of co-stimulatory signaling domains are provided in SEQ ID NOs: 45-46 (OX40), SEQ ID NO: 47-48 (4-1BB).

According to specific embodiments, any of the polypeptides disclosed herein can comprise a cytokine receptor signaling domain.

According to other specific embodiments, the polypeptides disclosed herein do not comprise a cytokine receptor signaling domain.

According to specific embodiments, any of the first and second polypeptides comprises a cytokine receptor signaling domain.

According to specific embodiments, the first polypeptide does not comprise a cytokine receptor signaling domain.

According to specific embodiments, the second polypeptide does not comprise a cytokine receptor signaling domain.

As used herein, the phrase “cytokine receptor signaling domain” refers to an amino acid sequence of a cytokine receptor capable of transmitting a stimulatory signal resulting in activation of the T cell.

Any known cytokine receptor signaling domain can be used with specific embodiments of the present invention. Non-limiting examples of cytokine receptor signaling domains include IL2rg that is the IL2 receptor common gamma chain (e.g. such as provided e.g. in SEQ ID NOs: 63-64), the Toll/IL1 receptor homology domain (TIR) that is the signaling domain of the myd88 receptor, TNF receptor intracellular domain (e.g. such as provided in SEQ ID NOs: 49-50), IL12-Rb1 intracellular domain (e.g. such as provided in SEQ ID NOs: 51-52), IL12-Rb1 intracellular domain (e.g. such as provided in SEQ ID NOs: 53-54), TL23 receptor intracellular domain (e.g. such as provided in SEQ ID NOs: 55-56), IFNγ receptor 1 intracellular domain (e.g. such as provided in SEQ ID NOs: 57-58), IFNγ receptor 2 intracellular domain (e.g. such as provided in SEQ ID NOs: 59-60), IL2Rb intracellular domain (e.g. such as provided in SEQ ID NOs: 61-62), IL1 receptor intracellular domain (e.g. such as provided in SEQ ID NOs: 65-66), IL1AcP receptor intracellular domain (e.g. such as provided in SEQ ID NOs: 67-68).

Any of the components comprised in a single polypeptide as described herein may be linked to each other directly of via a linker, each possibility represents a separate embodiment of the present invention.

According to specific embodiments, the second polypeptide does not comprise a linker between the extracellular ligand-binding domain of the Fc receptor and the amino acid sequence capable of recruiting the first polypeptide.

According to specific embodiments the second polypeptide comprises a linker between the extracellular ligand-binding domain of an Fc receptor and the amino acid sequence capable of recruiting the first polypeptide.

Any linker known in the art can be used with specific embodiments of the invention.

According to specific embodiments, the linker may be derived from naturally-occurring multi-domain proteins or is an empirical linker as described, for example, in Chichili et al., (2013), Protein Sci. 22(2): 153-167, Chen et al, (2013), Adv Drug Deliv Rev. 65(10): 1357-1369, the entire contents of which are hereby incorporated by reference. In some embodiments, the linker may be designed using linker designing databases and computer programs such as those described in Chen et al., (2013), Adv Drug Deliv Rev. 65(10): 1357-1369 and Crasto et al., (2000), Protein Eng. 13(5):309-312, the entire contents of which are hereby incorporated by reference.

According to specific embodiments, the linker is a synthetic linker.

According to specific embodiments, the linker is a polypeptide.

As the present inventor have discovered a novel subset of CD4⁺ T cells which expresses CD64 having function in tumor cell eradication (Examples 1 of the Examples section which follows), specific embodiments of the present invention contemplates T cells expressing CD64 and methods of obtaining and using such cells.

Thus, according to an aspect of the present invention, there is provided an isolated population of T cells comprising at least 80% T cells expressing endogenous CD64, said CD64 comprising an extracellular domain, a transmembrane domain and a cytoplasmic domain.

According to specific embodiments, the isolated population of T cells comprises at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% T cells expressing the endogenous CD64.

According to an additional or an alternative aspect of the present invention there is provided a method of isolating a T cell, the method comprising isolating a CD64+ T cell from a biological sample of a subject using an agent that binds a CD64 polypeptide or a polynucleotide encoding said CD64 polypeptide.

The CD64+ T cells can be obtained from any biological sample, such as peripheral blood, bone marrow, tissues such as spleen, lymph node, thymus, or tumor tissue. Selection of the biological sample would be evident to one of skill in the art.

According to specific embodiments, the biological sample is a peripheral blood sample.

There are several methods and reagents known to those skilled in the art for purifying T cells from a biological sample. Such methods are described for example in THE HANDBOOK OF EXPERIMENTAL IMMUNOLOGY, Volumes 1 to 4, (D. N. Weir, editor) and FLOW CYTOMETRY AND CELL SORTING (A. Radbruch, editor, Springer Verlag, 2000) and are further described hereinabove.

For isolating a CD64+ T cell, the biological sample is contacted with an agent that binds a CD64 polypeptide (e.g. an antibody) or a polynucleotide encoding said CD64 polypeptide (e.g. e.g. oligonucleotide probe or primer) and the cells are further selected using e.g. FACS sorter or magnetic cell separation techniques.

According to specific embodiments, the agent is an anti-CD64 antibody.

According to specific embodiments, following isolating of the CD64+ T cell, the cells are cultured, cloned, activated and/or genetically engineered.

According to specific embodiments, following isolating of the CD64+ T cell, a plurality of the cells is administered to a subject in need thereof.

Hence, according to an aspect of the present invention, there is provided a T cell clone expressing CD64, said CD64 comprises an extracellular domain, a transmembrane domain and a cytoplasmic domain.

According to another aspect of the present invention, there is provided a T cell genetically engineered to express CD64, said CD64 comprising an extracellular domain, a transmembrane domain and a cytoplasmic domain.

According to specific embodiments, the T cell is genetically engineered to express full length CD64.

According to other specific embodiments, the T cell is genetically engineered to express a functional fragment of CD64.

According to other specific embodiments, the T cell is genetically engineered to express a functional homolog of CD64.

According to specific embodiments, the T cell expressing CD64, either endogenously or exogenously, can be genetically engineered to express a polypeptide of interest.

According to specific embodiments, the T cell expressing CD64 is genetically engineered to express a polypeptide comprising an amino acid sequence of an FcRγ capable of transmitting an activating signal.

According to specific embodiments, the polypeptide comprising an amino acid sequence of FcRγ further comprises an amino acid sequence of a CD3ζ chain capable of transmitting an activating signal.

To express any of the disclosed exogenous polypeptides in T cells, a polynucleotide sequence encoding the polypeptide is preferably ligated into a nucleic acid construct suitable for T cell expression. Such a nucleic acid construct includes a promoter sequence for directing transcription of the polynucleotide sequence in the cell in a constitutive or inducible manner.

The nucleic acid construct (also referred to herein as an “expression vector”) of some embodiments of the invention includes additional sequences which render this vector suitable for replication and integration (e.g., shuttle vectors). In addition, a typical cloning vectors may also contain a transcription and translation initiation sequence, transcription and translation terminator and a polyadenylation signal. By way of example, such constructs will typically include a 5′ LTR, a tRNA binding site, a packaging signal, an origin of second-strand DNA synthesis, and a 3′ LTR or a portion thereof.

The nucleic acid construct of some embodiments of the invention typically includes or encodes a signal sequence for targeting the polypeptide to the cell surface. According to a specific embodiment, the signal sequence for this purpose is a mammalian signal sequence or the signal sequence of the polypeptide variants of some embodiments of the invention.

Eukaryotic promoters typically contain two types of recognition sequences, the TATA box and upstream promoter elements. The TATA box, located 25-30 base pairs upstream of the transcription initiation site, is thought to be involved in directing RNA polymerase to begin RNA synthesis. The other upstream promoter elements determine the rate at which transcription is initiated.

Preferably, the promoter utilized by the nucleic acid construct of some embodiments of the invention is active in the specific cell population transformed, i.e. T cells. Examples of T cell specific promoters include lymphoid specific promoters [Calame et al., (1988) Adv. Immunol. 43:235-275]; in particular promoters of T-cell receptors [Winoto et al., (1989) EMBO J. 8:729-733].

Enhancer elements can stimulate transcription up to 1,000 fold from linked homologous or heterologous promoters. Enhancers are active when placed downstream or upstream from the transcription initiation site. Many enhancer elements derived from viruses have a broad host range and are active in a variety of tissues. For example, the SV40 early gene enhancer is suitable for many cell types. Other enhancer/promoter combinations that are suitable for some embodiments of the invention include those derived from polyoma virus, human or murine cytomegalovirus (CMV), the long term repeat from various retroviruses such as murine leukemia virus, murine or Rous sarcoma virus and HIV. See, Enhancers and Eukaryotic Expression, Cold Spring Harbor Press, Cold Spring Harbor, N.Y. 1983, which is incorporated herein by reference.

In the construction of the expression vector, the promoter is preferably positioned approximately the same distance from the heterologous transcription start site as it is from the transcription start site in its natural setting. As is known in the art, however, some variation in this distance can be accommodated without loss of promoter function.

Polyadenylation sequences can also be added to the expression vector in order to increase the efficiency of mRNA translation. Two distinct sequence elements are required for accurate and efficient polyadenylation: GU or U rich sequences located downstream from the polyadenylation site and a highly conserved sequence of six nucleotides, AAUAAA, located 11-30 nucleotides upstream. Termination and polyadenylation signals that are suitable for some embodiments of the invention include those derived from SV40.

In addition to the elements already described, the expression vector of some embodiments of the invention may typically contain other specialized elements intended to increase the level of expression of cloned nucleic acids or to facilitate the identification of cells that carry the recombinant DNA. For example, a number of animal viruses contain DNA sequences that promote the extra chromosomal replication of the viral genome in permissive cell types. Plasmids bearing these viral replicons are replicated episomally as long as the appropriate factors are provided by genes either carried on the plasmid or with the genome of the host cell.

The vector may or may not include a eukaryotic replicon. If a eukaryotic replicon is present, then the vector is amplifiable in eukaryotic cells using the appropriate selectable marker. If the vector does not comprise a eukaryotic replicon, no episomal amplification is possible. Instead, the recombinant DNA integrates into the genome of the engineered cell, where the promoter directs expression of the desired nucleic acid.

The expression vector of some embodiments of the invention can further include additional polynucleotide sequences that allow, for example, the translation of several proteins from a single mRNA such as an internal ribosome entry site (IRES) or a self-cleavable peptide; and sequences for genomic integration of the promoter-chimeric polypeptide.

According to specific embodiments, the first and second polypeptides described herein are expressed from distinct constructs.

According to other specific embodiments, the first and second polypeptides described herein are expressed from a single construct in a bicistronic manner. Such an expression can be achieved by method well known in the art such as, but not limited to, using internal ribosome entry site (IRES) sequence and/or a nucleic acid sequence encoding a self-cleavable peptide e.g. a 2A peptide (e.g. P2A, T2A, E2A).

It will be appreciated that the individual elements comprised in the expression vector can be arranged in a variety of configurations. For example, enhancer elements, promoters and the like, and even the polynucleotide sequence(s) encoding the polypeptide can be arranged in a “head-to-tail” configuration, may be present as an inverted complement, or in a complementary configuration, as an anti-parallel strand. While such variety of configuration is more likely to occur with non-coding elements of the expression vector, alternative configurations of the coding sequence within the expression vector are also envisioned.

Examples for mammalian expression vectors include, but are not limited to, pcDNA3, pcDNA3.1(+/−), pGL3, pZeoSV2(+/−), pSecTag2, pDisplay, pEF/myc/cyto, pCMV/myc/cyto, pCR3.1, pSinRep5, DH26S, DHBB, pNMT1, pNMT41, pNMT81, which are available from Invitrogen, pCI which is available from Promega, pMbac, pPbac, pBK-RSV and pBK-CMV which are available from Strategene, pTRES which is available from Clontech, and their derivatives.

Expression vectors containing regulatory elements from eukaryotic viruses such as retroviruses can be also used. SV40 vectors include pSVT7 and pMT2. Vectors derived from bovine papilloma virus include pBV-1MTHA, and vectors derived from Epstein Bar virus include pHEBO, and p2O5. Other exemplary vectors include pMSG, pAV009/A⁺, pMTO10/A⁺, pMAMneo-5, baculovirus pDSVE, and any other vector allowing expression of proteins under the direction of the SV-40 early promoter, SV-40 later promoter, metallothionein promoter, murine mammary tumor virus promoter, Rous sarcoma virus promoter, polyhedrin promoter, or other promoters shown effective for expression in eukaryotic cells.

As described above, viruses are very specialized infectious agents that have evolved, in many cases, to elude host defense mechanisms. Typically, viruses infect and propagate in specific cell types. The targeting specificity of viral vectors utilizes its natural specificity to specifically target predetermined cell types and thereby introduce a recombinant gene into the infected cell. The ability to select suitable vectors for transforming T cells is well within the capabilities of the ordinary skilled artisan and as such no general description of selection consideration is provided herein.

Recombinant viral vectors are useful for in vivo expression of the polypeptides since they offer advantages such as lateral infection and targeting specificity. Lateral infection is inherent in the life cycle of, for example, retrovirus and is the process by which a single infected cell produces many progeny virions that bud off and infect neighboring cells. The result is that a large area becomes rapidly infected, most of which was not initially infected by the original viral particles. This is in contrast to vertical-type of infection in which the infectious agent spreads only through daughter progeny. Viral vectors can also be produced that are unable to spread laterally. This characteristic can be useful if the desired purpose is to introduce a specified gene into only a localized number of targeted cells.

Various methods can be used to introduce the expression vector of some embodiments of the invention into T cells. Such methods are generally described in Sambrook et al., Molecular Cloning: A Laboratory Manual, Cold Springs Harbor Laboratory, New York (1989, 1992), in Ausubel et al., Current Protocols in Molecular Biology, John Wiley and Sons, Baltimore, Md. (1989), Chang et al., Somatic Gene Therapy, CRC Press, Ann Arbor, Mich. (1995), Vega et al., Gene Targeting, CRC Press, Ann Arbor Mich. (1995), Vectors: A Survey of Molecular Cloning Vectors and Their Uses, Butterworths, Boston Mass. (1988) and Gilboa et al. [Biotechniques 4 (6): 504-512, 1986] and include, for example, stable or transient transfection, lipofection, electroporation and infection with recombinant viral vectors. In addition, see U.S. Pat. Nos. 5,464,764 and 5,487,992 for positive-negative selection methods.

Introduction of nucleic acids by viral infection offers several advantages over other methods such as lipofection and electroporation, since higher transfection efficiency can be obtained due to the infectious nature of viruses.

Currently preferred in vivo nucleic acid transfer techniques include transfection with viral or non-viral constructs, such as adenovirus, lentivirus, Herpes simplex I virus, or adeno-associated virus (AAV) and lipid-based systems. Useful lipids for lipid-mediated transfer of the gene are, for example, DOTMA, DOPE, and DC-Chol [Tonkinson et al., Cancer Investigation, 14(1): 54-65 (1996)]. The most preferred constructs for use in gene therapy are viruses, most preferably adenoviruses, AAV, lentiviruses, or retroviruses. A viral construct such as a retroviral construct includes at least one transcriptional promoter/enhancer or locus-defining element(s), or other elements that control gene expression by other means such as alternate splicing, nuclear RNA export, or post-translational modification of messenger. Such vector constructs also include a packaging signal, long terminal repeats (LTRs) or portions thereof, and positive and negative strand primer binding sites appropriate to the virus used, unless it is already present in the viral construct. In addition, such a construct typically includes a signal sequence for targeting the polypeptide to the desired site in a cell. Optionally, the construct may also include a signal that directs polyadenylation, as well as one or more restriction sites and a translation termination sequence. By way of example, such constructs will typically include a 5′ LTR, a tRNA binding site, a packaging signal, an origin of second-strand DNA synthesis, and a 3′ LTR or a portion thereof. Other vectors can be used that are non-viral, such as cationic lipids, polylysine, and dendrimers.

According to specific embodiments, the T cells can be freshly isolated, stored e.g., cryopreserved (i.e. frozen) at e.g. liquid nitrogen temperature at any stage for long periods of time (e.g., months, years) for future use; and cell lines.

Methods of cryopreservation are commonly known by one of ordinary skill in the art and are disclosed e.g. in International Patent Application Publication Nos. WO2007054160 and WO 2001039594 and US Patent Application Publication No. US20120149108.

According to specific embodiments, the T cells can be stored in a cell bank or a depository or storage facility.

Consequently, the present teachings further suggest the use of the T cells and the methods disclosed herein as, but not limited to, a source for adoptive T cells therapies for diseases that can benefit from activating immune cells against pathologic cells e.g. a hyper-proliferative disease; a disease associated with immune suppression and infections.

Thus, according to an aspect of the present invention, the T cells disclosed herein are for use in adoptive T cell therapy.

The T cells used according to specific embodiments of the present invention may be autologous or non-autologous; they can be syngeneic or non-syngeneic: allogeneic or xenogeneic to the subject; each possibility represents a separate embodiment of the present invention.

According to specific embodiments, the cells are autologous to said subject.

According to specific embodiments, the cells are non-autologous to said subject.

According to specific embodiments, the T cells described herein are cultured, expanded and/or activated ex-vivo prior to administration to the subject.

Methods of culturing, expanding and activating T cells are well known to the skilled in the art. For example, T cells may be activated ex vivo in the presence of one or more molecule such as, but not limited to, an anti-CD3 antibody, an anti-CD28 antibody, anti-CD3 and anti-CD28 coated beads (such as the CD3CD28 MACSiBeads obtained from Miltenyi Biotec), IL-2, phytohemagglutinin, an antigen-loaded antigen presenting cell [APC, e.g. dendritic cell], a peptide loaded recombinant MHC.

Since the T cells of specific embodiments of the present invention are activated upon binding of the extracellular ligand-binding domain of the FCγ receptor to an Fc ligand, they may be used for, but not limited to, treating diseases associated with pathologic cells in combination with a therapeutic composition comprising an Fc domain (e.g. antibody) which is directed for binding the pathologic cells.

Thus, according to an aspect of the present invention, there is provided a method of treating a disease associated with a pathologic cell in a subject treated with a therapeutic composition comprising an Fc domain, said therapeutic composition being specific for said pathologic cell, the method comprising administering to the subject a therapeutically effective amount of the T cells or the population of T cells disclosed herein, thereby treating the disease in the subject.

According to an additional or an alternative aspect of the present invention, there is provided the T cells or the population of T cells disclosed herein, for use in treating a disease associated with a pathologic cell in a subject treated with a therapeutic composition comprising an Fc domain, said therapeutic composition being specific for said pathologic cell.

According to an additional or an alternative aspect of the present invention, there is provided a method of treating a disease associated with a pathologic cell in a subject in need thereof, the method comprising administering to the subject a therapeutically effective amount of the T cells or the population of T cells disclosed herein; and a therapeutic composition comprising an Fc domain, said therapeutic composition being specific for said pathologic cell, thereby treating the disease in the subject.

According to an additional or an alternative aspect of the present invention, there is provided the T cells or the population of T cells disclosed herein; and a therapeutic composition comprising an Fc domain, for use in treating a disease associated with a pathologic cell in a subject in need thereof, wherein said therapeutic composition is specific for said pathologic cell.

As used herein, the term “subject” or “subject in need thereof” includes mammals, preferably human beings at any age or gender. The subject may be healthy or showing preliminary signs of a pathology, e.g. cancer. This term also encompasses individuals who are at risk to develop the pathology.

As used herein the term “treating” refers to curing, reversing, attenuating, alleviating, minimizing, suppressing or halting the deleterious effects of a disease or disorder (e.g. cancer). Those of skill in the art will understand that various methodologies and assays can be used to assess the development of a pathology, and similarly, various methodologies and assays may be used to assess the reduction, remission or regression of a pathology (e.g. a malignancy), as discussed below.

As used herein, the term “preventing” refers to keeping a disease, disorder or condition from occurring in a subject who may be at risk for the disease, but has not yet been diagnosed as having the disease.

As used herein the phrase, “disease associated with a pathologic cell” means that pathologic cells drive onset and/or progression of the disease.

According to specific embodiments, the disease can benefit from activating the immune cells of the subject.

As used herein the phrase “a disease that can benefit from activating immune cells” refers to diseases in which the subject's immune response activity may be sufficient to at least ameliorate symptoms of the disease or delay onset of symptoms, however for any reason the activity of the subject's immune response in doing so is less than optimal.

Non-limiting examples of diseases treated by some embodiments of the invention include hyper-proliferative diseases, diseases associated with immune suppression, immunosuppression caused by medication (e.g. mTOR inhibitors, calcineurin inhibitor, steroids) and infections.

According to specific embodiments, the disease comprises an infection.

As used herein, the term “infection” or “infectious disease” refers to a disease induced by a pathogen. Specific examples of pathogens include, viral pathogens, bacterial pathogens e.g., intracellular mycobacterial pathogens (such as, for example, Mycobacterium tuberculosis), intracellular bacterial pathogens (such as, for example, Listeria monocytogenes), or intracellular protozoan pathogens (such as, for example, Leishmania and Trypanosoma).

Specific types of viral pathogens causing infectious diseases include, but are not limited to, retroviruses, circoviruses, parvoviruses, papovaviruses, adenoviruses, herpesviruses, iridoviruses, poxviruses, hepadnaviruses, picornaviruses, caliciviruses, togaviruses, flaviviruses, reoviruses, orthomyxoviruses, paramyxoviruses, rhabdoviruses, bunyaviruses, coronaviruses, arenaviruses, and filoviruses.

Specific examples of viral infections which may be treated according to specific embodiments of the present invention include, but are not limited to, human immunodeficiency virus (HIV)-induced acquired immunodeficiency syndrome (AIDS), influenza, rhinoviral infection, viral meningitis, Epstein-Barr virus (EBV) infection, hepatitis A, B or C virus infection, measles, papilloma virus infection/warts, cytomegalovirus (CMV) infection, Herpes simplex virus infection, yellow fever, Ebola virus infection, rabies, etc.

According to specific embodiments, the disease comprises a hyper-proliferative disease.

According to specific embodiments, the hyper-proliferative disease comprises sclerosis, fibrosis, Idiopathic pulmonary fibrosis, psoriasis, systemic sclerosis/scleroderma, primary biliary cholangitis, primary sclerosing cholangitis, liver fibrosis, prevention of radiation-induced pulmonary fibrosis, myelofibrosis or retroperitoneal fibrosis.

According to other specific embodiments, the hyper-proliferative disease comprises cancer.

Thus, according to specific embodiments the pathologic cell is a cancerous cell.

Cancers which may be treated by some embodiments of the invention can be any solid or non-solid tumor, cancer metastasis and/or a pre-cancer.

According to specific embodiments, the cancer is a malignant cancer.

Examples of cancer include but are not limited to, carcinoma, blastoma, sarcoma and lymphoma. More particular examples of such cancers include, but are not limited to, tumors of the gastrointestinal tract (colon carcinoma, rectal carcinoma, colorectal carcinoma, colorectal cancer, colorectal adenoma, hereditary nonpolyposis type 1, hereditary nonpolyposis type 2, hereditary nonpolyposis type 3, hereditary nonpolyposis type 6; colorectal cancer, hereditary nonpolyposis type 7, small and/or large bowel carcinoma, esophageal carcinoma, tylosis with esophageal cancer, stomach carcinoma, pancreatic carcinoma, pancreatic endocrine tumors), endometrial carcinoma, dermatofibrosarcoma protuberans, gallbladder carcinoma, Biliary tract tumors, prostate cancer, prostate adenocarcinoma, renal cancer (e.g., Wilms' tumor type 2 or type 1), liver cancer (e.g., hepatoblastoma, hepatocellular carcinoma, hepatocellular cancer), bladder cancer, embryonal rhabdomyosarcoma, germ cell tumor, trophoblastic tumor, testicular germ cells tumor, immature teratoma of ovary, uterine, epithelial ovarian, sacrococcygeal tumor, choriocarcinoma, placental site trophoblastic tumor, epithelial adult tumor, ovarian carcinoma, serous ovarian cancer, ovarian sex cord tumors, cervical carcinoma, uterine cervix carcinoma, small-cell and non-small cell lung carcinoma, nasopharyngeal, breast carcinoma (e.g., ductal breast cancer, invasive intraductal breast cancer, sporadic; breast cancer, susceptibility to breast cancer, type 4 breast cancer, breast cancer-1, breast cancer-3; breast-ovarian cancer), squamous cell carcinoma (e.g., in head and neck), neurogenic tumor, astrocytoma, ganglioblastoma, neuroblastoma, lymphomas (e.g., Hodgkin's disease, non-Hodgkin's lymphoma, B cell, Burkitt, cutaneous T cell, histiocytic, lymphoblastic, T cell, thymic), gliomas, adenocarcinoma, adrenal tumor, hereditary adrenocortical carcinoma, brain malignancy (tumor), various other carcinomas (e.g., bronchogenic large cell, ductal, Ehrlich-Lettre ascites, epidermoid, large cell, Lewis lung, medullary, mucoepidermoid, oat cell, small cell, spindle cell, spinocellular, transitional cell, undifferentiated, carcinosarcoma, choriocarcinoma, cystadenocarcinoma), ependimoblastoma, epithelioma, erythroleukemia (e.g., Friend, lymphoblast), fibrosarcoma, giant cell tumor, glial tumor, glioblastoma (e.g., multiforme, astrocytoma), glioma hepatoma, heterohybridoma, heteromyeloma, histiocytoma, hybridoma (e.g., B cell), hypernephroma, insulinoma, islet tumor, keratoma, leiomyoblastoma, leiomyosarcoma, leukemia (e.g., acute lymphatic, acute lymphoblastic, acute lymphoblastic pre-B cell, acute lymphoblastic T cell leukemia, acute-megakaryoblastic, monocytic, acute myelogenous, acute myeloid, acute myeloid with eosinophilia, B cell, basophilic, chronic myeloid, chronic, B cell, eosinophilic, Friend, granulocytic or myelocytic, hairy cell, lymphocytic, megakaryoblastic, monocytic, monocytic-macrophage, myeloblastic, myeloid, myelomonocytic, plasma cell, pre-B cell, promyelocytic, subacute, T cell, lymphoid neoplasm, predisposition to myeloid malignancy, acute nonlymphocytic leukemia), lymphosarcoma, melanoma, mammary tumor, mastocytoma, medulloblastoma, mesothelioma, metastatic tumor, monocyte tumor, multiple myeloma, myelodysplastic syndrome, myeloma, nephroblastoma, nervous tissue glial tumor, nervous tissue neuronal tumor, neurinoma, neuroblastoma, oligodendroglioma, osteochondroma, osteomyeloma, osteosarcoma (e.g., Ewing's), papilloma, transitional cell, pheochromocytoma, pituitary tumor (invasive), plasmacytoma, retinoblastoma, rhabdomyosarcoma, sarcoma (e.g., Ewing's, histiocytic cell, Jensen, osteogenic, reticulum cell), schwannoma, subcutaneous tumor, teratocarcinoma (e.g., pluripotent), teratoma, testicular tumor, thymoma and trichoepithelioma, gastric cancer, fibrosarcoma, glioblastoma multiforme; multiple glomus tumors, Li-Fraumeni syndrome, liposarcoma, lynch cancer family syndrome II, male germ cell tumor, mast cell leukemia, medullary thyroid, multiple meningioma, endocrine neoplasia myxosarcoma, paraganglioma, familial nonchromaffin, pilomatricoma, papillary, familial and sporadic, rhabdoid predisposition syndrome, familial, rhabdoid tumors, soft tissue sarcoma, and Turcot syndrome with glioblastoma.

According to specific embodiments, the cancer is a pre-malignant cancer.

Pre-cancers are well characterized and known in the art (refer, for example, to Berman J J. and Henson D E., 2003. Classifying the pre-cancers: a metadata approach. BMC Med Inform Decis Mak. 3:8). Examples of pre-cancers include, but are not limited to, acquired small pre-cancers, acquired large lesions with nuclear atypia, precursor lesions occurring with inherited hyperplastic syndromes that progress to cancer, and acquired diffuse hyperplasias and diffuse metaplasias. Non-limiting examples of small pre-cancers include HGSIL (High grade squamous intraepithelial lesion of uterine cervix), AIN (anal intraepithelial neoplasia), dysplasia of vocal cord, aberrant crypts (of colon), PIN (prostatic intraepithelial neoplasia).

Non-limiting examples of acquired large lesions with nuclear atypia include tubular adenoma, AILD (angioimmunoblastic lymphadenopathy with dysproteinemia), atypical meningioma, gastric polyp, large plaque parapsoriasis, myelodysplasia, papillary transitional cell carcinoma in-situ, refractory anemia with excess blasts, and Schneiderian papilloma. Non-limiting examples of precursor lesions occurring with inherited hyperplastic syndromes that progress to cancer include atypical mole syndrome, C cell adenomatosis and MEA. Non-limiting examples of acquired diffuse hyperplasias and diffuse metaplasias include Paget's disease of bone and ulcerative colitis.

According to specific embodiments, the cancer is selected from the group consisting of melanoma, adenocarcinoma, mammary carcinoma, colon cancer, ovarian cancer, lung cancer and B-cell lymphoma.

According to specific embodiments, the cancer is selected from the group consisting of melanoma, adenocarcinoma and mammary carcinoma.

According to specific embodiments, the cancer is selected from the group consisting of melanoma, adenocarcinoma and mammary carcinoma.

According to specific embodiments, the cancer or the cancerous cell expresses a marker selected from the group consisting of PDL-1, E-Cadherin, CD19, MUC1, TRP-1 and TRP-2.

According to specific embodiments, the cancer or the cancerous cell expresses PDL-1.

As mentioned, according to specific embodiments, the T cells are administered to the subject in combination with a therapeutic composition comprising an Fc domain (e.g. an antibody).

The administration of the T cells and the administration of the therapeutic composition comprising the Fc domain can be effected in the same route or in separate routes.

The administration of the T cells may be following or concomitant with the therapeutic composition comprising the Fc domain.

According to specific embodiments, the T cells disclosed herein are administered to the subject following treatment with the therapeutic composition comprising the Fc domain.

According to other specific embodiments, the T cells disclosed herein are administered to the subject concomitantly with the therapeutic composition comprising the Fc domain.

Multiple rounds of administration of the T cells and multiple doses of the therapeutic composition comprising the Fc domain can be administered. Thus, according to specific embodiments, administering the T cells disclosed herein is effected following at least one administration of the therapeutic composition comprising the Fc domain. According to specific embodiments, administering the cells disclosed herein is effected in a sequential order with the treatment with the therapeutic composition comprising the Fc domain.

According to specific embodiments, the therapeutic composition comprising the Fc domain is specific for a pathologic cell, i.e. binds an antigen overexpressed or solely expressed by a pathologic (e.g. cancerous) cell as compared to a non-pathologic cell.

Therapeutic compositions comprising Fc domains specific for pathologic cells are well known in the art and include, but not limited to, Fc-fusion proteins and antibodies.

According to specific embodiments, the Fc domain is of an IgG antibody.

As used herein the term, “Fc-fusion protein” refers to a molecule comprising an amino acid sequence capable of binding a pathologic cell (e.g. a ligand of a receptor expressed on a pathologic cell) combined with an Fc domain of an antibody.

Selection of the Fc-fusion protein used is well within the capability of those skilled in the art, and depends on the type of the disease and e.g. the receptors expressed by the pathologic cells associated with the pathology.

Non-limiting examples of Fc-fusion proteins that can be used with specific embodiments are disclosed in Weidle et al. Cancer Genomics and Proteomics (2012) 9(6): 357-372; and Sioud et al. Molecular Therapy—Methods & Clinical Development (2015) 2, 15043, the contents of which is fully incorporated herein by reference.

The term “antibody” as used in this invention includes intact molecules as well as functional fragments thereof (that are capable of binding to an epitope of an antigen). According to specific embodiments, the antibody comprises an Fc domain.

According to specific embodiments, the antibody is an IgG antibody (e.g. IgG1, IgG2, IgG3, IgG4).

According to a specific embodiment the antibody isotype is IgG1 or IgG3.

Selection of the antibody used is well within the capability of those skilled in the art, and depends on the type of the disease and the antigens expressed by the pathologic cells associated with the pathology.

According to specific embodiments, the antibody binds an antigen overexpressed or solely expressed by tumor cells.

According to some embodiments of the invention, the antibody is selected from the group consisting of Atezolizumab, Avelumab, Alemtuzumab, Cetuximab, Panitumumab, Nimotuzumab, Rituximab, Gatipotuzumab (previously known as PankoMab-GEX®), Trastuzumab, Alemtuzumab, Bevacizumab, Ofatumumab, Pertuzumab, ofatumumab, obinutuzumab and IVIG.

According to specific embodiments, the antibody is selected from the group consisting of Atezolizumab, Rituximab, Cetuximab, Gatipotuzumab and IVIG.

According to specific embodiments, the antibody is an anti-PDL-1.

According to specific embodiments, the cancerous cell expresses PDL-1 and the antibody is an anti-PDL-1.

According to specific embodiments, the antibody is Atezolizumab.

According to specific embodiments, the T cells and the therapeutic compositions disclosed herein can be administered to a subject in combination with other established or experimental therapeutic regimen to treat a disease associated with pathologic cells (e.g. cancer) including, but not limited to analgesics, chemotherapeutic agents, radiotherapeutic agents, cytotoxic therapies (conditioning), hormonal therapy and other treatment regimens (e.g., surgery) which are well known in the art.

The T cells disclosed herein and/or the therapeutic compositions disclosed herein can be administered to the subject per se, or in a pharmaceutical composition where it is mixed with suitable carriers or excipients.

As used herein a “pharmaceutical composition” refers to a preparation of one or more of the active ingredients described herein with other chemical components such as physiologically suitable carriers and excipients. The purpose of a pharmaceutical composition is to facilitate administration of a compound to an organism.

Herein the term “active ingredient” refers to the T cells and/or the antibodies accountable for the biological effect.

Thus, according to specific embodiments, the T cells are the active ingredient in the formulation.

Hereinafter, the phrases “physiologically acceptable carrier” and “pharmaceutically acceptable carrier” which may be interchangeably used refer to a carrier or a diluent that does not cause significant irritation to an organism and does not abrogate the biological activity and properties of the administered compound. An adjuvant is included under these phrases.

Herein the term “excipient” refers to an inert substance added to a pharmaceutical composition to further facilitate administration of an active ingredient. Examples, without limitation, of excipients include calcium carbonate, calcium phosphate, various sugars and types of starch, cellulose derivatives, gelatin, vegetable oils and polyethylene glycols.

Techniques for formulation and administration of drugs may be found in “Remington's Pharmaceutical Sciences,” Mack Publishing Co., Easton, PA, latest edition, which is incorporated herein by reference.

Suitable routes of administration may, for example, include oral, rectal, transmucosal, especially transnasal, intestinal or parenteral delivery, including intramuscular, intradermal, subcutaneous and intramedullary injections as well as intrathecal, direct intraventricular, intracardiac, e.g., into the right or left ventricular cavity, into the common coronary artery, intravenous, intraperitoneal, intranasal, or intraocular injections.

Conventional approaches for drug delivery to the central nervous system (CNS) include: neurosurgical strategies (e.g., intracerebral injection or intracerebroventricular infusion); molecular manipulation of the agent (e.g., production of a chimeric fusion protein that comprises a transport peptide that has an affinity for an endothelial cell surface molecule in combination with an agent that is itself incapable of crossing the BBB) in an attempt to exploit one of the endogenous transport pathways of the BBB; pharmacological strategies designed to increase the lipid solubility of an agent (e.g., conjugation of water-soluble agents to lipid or cholesterol carriers); and the transitory disruption of the integrity of the BBB by hyperosmotic disruption (resulting from the infusion of a mannitol solution into the carotid artery or the use of a biologically active agent such as an angiotensin peptide). However, each of these strategies has limitations, such as the inherent risks associated with an invasive surgical procedure, a size limitation imposed by a limitation inherent in the endogenous transport systems, potentially undesirable biological side effects associated with the systemic administration of a chimeric molecule comprised of a carrier motif that could be active outside of the CNS, and the possible risk of brain damage within regions of the brain where the BBB is disrupted, which renders it a suboptimal delivery method.

Alternately, one may administer the pharmaceutical composition in a local rather than systemic manner, for example, via injection of the pharmaceutical composition directly into a tissue region of a patient.

According to a specific embodiment, the T cells of the invention or the pharmaceutical composition comprising same is administered via an IV route.

Pharmaceutical compositions of some embodiments of the invention may be manufactured by processes well known in the art, e.g., by means of conventional mixing, dissolving, granulating, dragee-making, levigating, emulsifying, encapsulating, entrapping or lyophilizing processes.

Pharmaceutical compositions for use in accordance with some embodiments of the invention thus may be formulated in conventional manner using one or more physiologically acceptable carriers comprising excipients and auxiliaries, which facilitate processing of the active ingredients into preparations which, can be used pharmaceutically. Proper formulation is dependent upon the route of administration chosen.

For injection, the active ingredients of the pharmaceutical composition may be formulated in aqueous solutions, preferably in physiologically compatible buffers such as Hank's solution, Ringer's solution, or physiological salt buffer. For transmucosal administration, penetrants appropriate to the barrier to be permeated are used in the formulation. Such penetrants are generally known in the art.

For oral administration, the pharmaceutical composition can be formulated readily by combining the active compounds with pharmaceutically acceptable carriers well known in the art. Such carriers enable the pharmaceutical composition to be formulated as tablets, pills, dragees, capsules, liquids, gels, syrups, slurries, suspensions, and the like, for oral ingestion by a patient. Pharmacological preparations for oral use can be made using a solid excipient, optionally grinding the resulting mixture, and processing the mixture of granules, after adding suitable auxiliaries if desired, to obtain tablets or dragee cores. Suitable excipients are, in particular, fillers such as sugars, including lactose, sucrose, mannitol, or sorbitol; cellulose preparations such as, for example, maize starch, wheat starch, rice starch, potato starch, gelatin, gum tragacanth, methyl cellulose, hydroxypropylmethyl-cellulose, sodium carbomethylcellulose; and/or physiologically acceptable polymers such as polyvinylpyrrolidone (PVP). If desired, disintegrating agents may be added, such as cross-linked polyvinyl pyrrolidone, agar, or alginic acid or a salt thereof such as sodium alginate.

Dragee cores are provided with suitable coatings. For this purpose, concentrated sugar solutions may be used which may optionally contain gum arabic, talc, polyvinyl pyrrolidone, carbopol gel, polyethylene glycol, titanium dioxide, lacquer solutions and suitable organic solvents or solvent mixtures. Dyestuffs or pigments may be added to the tablets or dragee coatings for identification or to characterize different combinations of active compound doses.

Pharmaceutical compositions which can be used orally, include push-fit capsules made of gelatin as well as soft, sealed capsules made of gelatin and a plasticizer, such as glycerol or sorbitol. The push-fit capsules may contain the active ingredients in admixture with filler such as lactose, binders such as starches, lubricants such as talc or magnesium stearate and, optionally, stabilizers. In soft capsules, the active ingredients may be dissolved or suspended in suitable liquids, such as fatty oils, liquid paraffin, or liquid polyethylene glycols. In addition, stabilizers may be added. All formulations for oral administration should be in dosages suitable for the chosen route of administration.

For buccal administration, the compositions may take the form of tablets or lozenges formulated in conventional manner.

For administration by nasal inhalation, the active ingredients for use according to some embodiments of the invention are conveniently delivered in the form of an aerosol spray presentation from a pressurized pack or a nebulizer with the use of a suitable propellant, e.g., dichlorodifluoromethane, trichlorofluoromethane, dichloro-tetrafluoroethane or carbon dioxide. In the case of a pressurized aerosol, the dosage unit may be determined by providing a valve to deliver a metered amount. Capsules and cartridges of, e.g., gelatin for use in a dispenser may be formulated containing a powder mix of the compound and a suitable powder base such as lactose or starch.

The pharmaceutical composition described herein may be formulated for parenteral administration, e.g., by bolus injection or continuous infusion. Formulations for injection may be presented in unit dosage form, e.g., in ampoules or in multidose containers with optionally, an added preservative. The compositions may be suspensions, solutions or emulsions in oily or aqueous vehicles, and may contain formulatory agents such as suspending, stabilizing and/or dispersing agents.

Pharmaceutical compositions for parenteral administration include aqueous solutions of the active preparation in water-soluble form. Additionally, suspensions of the active ingredients may be prepared as appropriate oily or water based injection suspensions. Suitable lipophilic solvents or vehicles include fatty oils such as sesame oil, or synthetic fatty acids esters such as ethyl oleate, triglycerides or liposomes. Aqueous injection suspensions may contain substances, which increase the viscosity of the suspension, such as sodium carboxymethyl cellulose, sorbitol or dextran. Optionally, the suspension may also contain suitable stabilizers or agents which increase the solubility of the active ingredients to allow for the preparation of highly concentrated solutions.

Alternatively, the active ingredient may be in powder form for constitution with a suitable vehicle, e.g., sterile, pyrogen-free water based solution, before use.

The pharmaceutical composition of some embodiments of the invention may also be formulated in rectal compositions such as suppositories or retention enemas, using, e.g., conventional suppository bases such as cocoa butter or other glycerides.

Alternative embodiments include depots providing sustained release or prolonged duration of activity of the active ingredient in the subject, as are well known in the art.

Pharmaceutical compositions suitable for use in context of some embodiments of the invention include compositions wherein the active ingredients are contained in an amount effective to achieve the intended purpose. More specifically, a therapeutically effective amount means an amount of active ingredients effective to prevent, alleviate or ameliorate symptoms of a disorder (e.g., cancer) or prolong the survival of the subject being treated.

Determination of a therapeutically effective amount is well within the capability of those skilled in the art, especially in light of the detailed disclosure provided herein.

For any preparation used in the methods of the invention, the therapeutically effective amount or dose can be estimated initially from in vitro and cell culture assays. For example, a dose can be formulated in animal models to achieve a desired concentration or titer. Such information can be used to more accurately determine useful doses in humans.

Toxicity and therapeutic efficacy of the active ingredients described herein can be determined by standard pharmaceutical procedures in vitro, in cell cultures or experimental animals. The data obtained from these in vitro and cell culture assays and animal studies can be used in formulating a range of dosage for use in human. The dosage may vary depending upon the dosage form employed and the route of administration utilized. The exact formulation, route of administration and dosage can be chosen by the individual physician in view of the patient's condition. (See e.g., Fingl, et al., 1975, in “The Pharmacological Basis of Therapeutics”, Ch. 1 p. 1).

Dosage amount and interval may be adjusted individually to provide levels of the active ingredient are sufficient to induce or suppress the biological effect (minimal effective concentration, MEC). The MEC will vary for each preparation, but can be estimated from in vitro data. Dosages necessary to achieve the MEC will depend on individual characteristics and route of administration. Detection assays can be used to determine plasma concentrations.

Depending on the severity and responsiveness of the condition to be treated, dosing can be of a single or a plurality of administrations, with course of treatment lasting from several days to several weeks or until cure is effected or diminution of the disease state is achieved.

The amount of a composition to be administered will, of course, be dependent on the subject being treated, the severity of the affliction, the manner of administration, the judgment of the prescribing physician, etc.

Compositions of some embodiments of the invention may, if desired, be presented in a pack or dispenser device, such as an FDA approved kit, which may contain one or more unit dosage forms containing the active ingredient. The pack may, for example, comprise metal or plastic foil, such as a blister pack. The pack or dispenser device may be accompanied by instructions for administration. The pack or dispenser may also be accommodated by a notice associated with the container in a form prescribed by a governmental agency regulating the manufacture, use or sale of pharmaceuticals, which notice is reflective of approval by the agency of the form of the compositions or human or veterinary administration. Such notice, for example, may be of labeling approved by the U.S. Food and Drug Administration for prescription drugs or of an approved product insert. Compositions comprising a preparation of the invention formulated in a compatible pharmaceutical carrier may also be prepared, placed in an appropriate container, and labeled for treatment of an indicated condition, as is further detailed above.

According to another aspect of the present invention there is provided an article of manufacture comprising a packaging material packaging the T cells or the population of T cells disclosed herein and a therapeutic composition comprising an Fc domain.

According to specific embodiments, the article of manufacture is identified for the treatment of a disease associated with a pathologic cell (e.g. cancer).

According to specific embodiments, the T cells or the population of T cells disclosed herein; and the therapeutic composition comprising the Fc domain are packaged in separate containers.

According to specific embodiments, the T cells or the population of T cells disclosed herein; and the therapeutic composition comprising an Fc domain are packaged in a co-formulation.

As used herein the term “about” refers to +10%

The terms “comprises”, “comprising”, “includes”, “including”, “having” and their conjugates mean “including but not limited to”.

The term “consisting of” means “including and limited to”.

The term “consisting essentially of” means that the composition, method or structure may include additional ingredients, steps and/or parts, but only if the additional ingredients, steps and/or parts do not materially alter the basic and novel characteristics of the claimed composition, method or structure.

As used herein, the singular form “a”, “an” and “the” include plural references unless the context clearly dictates otherwise. For example, the term “a compound” or “at least one compound” may include a plurality of compounds, including mixtures thereof.

Throughout this application, various embodiments of this invention may be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

Whenever a numerical range is indicated herein, it is meant to include any cited numeral (fractional or integral) within the indicated range. The phrases “ranging/ranges between” a first indicate number and a second indicate number and “ranging/ranges from” a first indicate number “to” a second indicate number are used herein interchangeably and are meant to include the first and second indicated numbers and all the fractional and integral numerals therebetween.

As used herein the term “method” refers to manners, means, techniques and procedures for accomplishing a given task including, but not limited to, those manners, means, techniques and procedures either known to, or readily developed from known manners, means, techniques and procedures by practitioners of the chemical, pharmacological, biological, biochemical and medical arts.

When reference is made to particular sequence listings, such reference is to be understood to also encompass sequences that substantially correspond to its complementary sequence as including minor sequence variations, resulting from, e.g., sequencing errors, cloning errors, or other alterations resulting in base substitution, base deletion or base addition, provided that the frequency of such variations is less than 1 in 50 nucleotides, alternatively, less than 1 in 100 nucleotides, alternatively, less than 1 in 200 nucleotides, alternatively, less than 1 in 500 nucleotides, alternatively, less than 1 in 1000 nucleotides, alternatively, less than 1 in 5,000 nucleotides, alternatively, less than 1 in 10,000 nucleotides.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable subcombination or as suitable in any other described embodiment of the invention. Certain features described in the context of various embodiments are not to be considered essential features of those embodiments, unless the embodiment is inoperative without those elements.

Various embodiments and aspects of the present invention as delineated hereinabove and as claimed in the claims section below find experimental support in the following examples.

### EXAMPLES

Reference is now made to the following examples, which together with the above descriptions illustrate some embodiments of the invention in a non-limiting fashion.

Generally, the nomenclature used herein and the laboratory procedures utilized in the present invention include molecular, biochemical, microbiological and recombinant DNA techniques. Such techniques are thoroughly explained in the literature. See, for example, “Molecular Cloning: A laboratory Manual” Sambrook et al., (1989); “Current Protocols in Molecular Biology” Volumes I-III Ausubel, R. M., ed. (1994); Ausubel et al., “Current Protocols in Molecular Biology”, John Wiley and Sons, Baltimore, Maryland (1989); Perbal, “A Practical Guide to Molecular Cloning”, John Wiley & Sons, New York (1988); Watson et al., “Recombinant DNA”, Scientific American Books, New York; Birren et al. (eds) “Genome Analysis: A Laboratory Manual Series”, Vols. 1-4, Cold Spring Harbor Laboratory Press, New York (1998); methodologies as set forth in U.S. Pat. Nos. 4,666,828; 4,683,202; 4,801,531; 5,192,659 and 5,272,057; “Cell Biology: A Laboratory Handbook”, Volumes I-III Cellis, J. E., ed. (1994); “Culture of Animal Cells—A Manual of Basic Technique” by Freshney, Wiley-Liss, N. Y. (1994), Third Edition; “Current Protocols in Immunology” Volumes I-III Coligan J. E., ed. (1994); Stites et al. (eds), “Basic and Clinical Immunology” (8th Edition), Appleton & Lange, Norwalk, CT (1994); Mishell and Shiigi (eds), “Selected Methods in Cellular Immunology”, W. H. Freeman and Co., New York (1980); available immunoassays are extensively described in the patent and scientific literature, see, for example, U.S. Pat. Nos. 3,791,932; 3,839,153; 3,850,752; 3,850,578; 3,853,987; 3,867,517; 3,879,262; 3,901,654; 3,935,074; 3,984,533; 3,996,345; 4,034,074; 4,098,876; 4,879,219; 5,011,771 and 5,281,521; “Oligonucleotide Synthesis” Gait, M. J., ed. (1984); “Nucleic Acid Hybridization” Hames, B. D., and Higgins S. J., eds. (1985); “Transcription and Translation” Hames, B. D., and Higgins S. J., eds. (1984); “Animal Cell Culture” Freshney, R. I., ed. (1986); “Immobilized Cells and Enzymes” IRL Press, (1986); “A Practical Guide to Molecular Cloning” Perbal, B., (1984) and “Methods in Enzymology” Vol. 1-317, Academic Press; “PCR Protocols: A Guide To Methods And Applications”, Academic Press, San Diego, CA (1990); Marshak et al., “Strategies for Protein Purification and Characterization—A Laboratory Course Manual” CSHL Press (1996); all of which are incorporated by reference as if fully set forth herein. Other general references are provided throughout this document. The procedures therein are believed to be well known in the art and are provided for the convenience of the reader. All the information contained therein is incorporated herein by reference.

### Example 1

A Subset of Cd4+ T Cells Functional in Inducing Direct Tumor Lysis in Combination with Anti-Cancer Antibodies Expresses Fcγ Receptors

### Materials and Methods

Mice—Wild-type (WT) C57BL/6 and Balb/cOlaHsd mice were obtained from Envigo (Jerusalem, Israel), and from Jackson Laboratories (Bar-Harbor, ME, USA). T cell deficient mice B6.Cg-Rag1^{tm1Mom} and TCR transgenic mice Tyrp1B-w Tg(Tcrα,Tcrβ)9Rest/J were purchased from Jackson Laboratory. B6.Cg-Tg(Tcrα,Tcrβ)425Cbn/J were purchased from Jackson Laboratory, or kindly provided by Professor Ronen Alon at the Weizmann Institute. All mice were housed in an American Association for the Accreditation of Laboratory Animal Care-accredited animal facility and maintained in specific pathogen-free conditions. Male and female 8-12 weeks old mice were used in all experiments. All animal experiments were approved by the Tel-Aviv University or the Stanford University Institutional Animal Care and Use Committees.

Cell lines—B16F10 cells (CRL-6475) and 4T1 (CRL-2539) cells were purchased from the ATCC, and HEK-293FT were purchased from ThermoFisher Scientific (Waltham, MA). Cells were cultured in DMEM (GIBCO) supplemented with 10% heat-inactivated FBS (Biological Industries, Israel), 2 mM L-glutamine, and 100 μg/mL penicillin/streptomycin (GIBCO) under standard conditions. Cells were routinely tested for mycoplasma using EZ-PCR Mycoplasma Test Kit (Biological Industries, Israel) according to manufacturer's instructions.

T cell isolation—All tissue preparations were performed simultaneously from each individual mouse following euthanasia by CO₂ inhalation. For isolation of T cells from lymphoid organs: spleen, lymph nodes and thymus were removed from euthanized mice and mashed through 70 μM cell strainer (Gibco, Thermo Fisher Scientific, Waltham, MA). Following, cells were washed by centrifugation in 2,000 rpm 5 min 4-8° C. For isolation of Tumor-infiltrating T cells: Tumors were enzymatically digested with 2,000 U/ml of DNase I and 2 mg/mL collagenase IV (both from Sigma Aldrich, Merck, Israel) in HBSS for 30 minutes 37° C. with magnetic stirrer (400 rpm). Following, cells were washed by centrifugation in 2,000 rpm 5 minutes 4-8° C. For isolation of T cells from peripheral blood: peripheral blood was collected via the posterior vena cava prior to perfusion of the animal and transferred into sodium heparin-coated vacuum tubes prior to 1:1 dilution in FACS buffer (Hanks' Balanced Salt solution, 2% FSC, 0.05 mM EDTA). Lymphocytes were enriched on Ficoll®-Paque Premium (Sigma-Aldrich) gradient and collected PBMC were washed twice with FACS buffer. For all tissues, cells were incubated with anti-CD4, or anti-CD8 magnetic beads (MojoSort™ Nanobeads, BioLegend, Carlsbad, CA) according to manufacturer's instruction and further sorted by FACSAriaII as FCS^{lo}/SSC^{lo}/TCRβ⁺/MHCII^{neg} cells.

T cell culture and expansion—T cells were cultured in RPMI-1640 supplemented with 1% Pen-Strep, 10% heat inactivated FBS, 1% Sodium pyruvate, 1% MEM-Eagle non-essential amino acids, 1% Insulin-Transferrin-Selenium, and 50 μM β-mercaptoethanol. For T cell expansion, culture dishes were pre-coated with 0.5 μg/ml anti-CD3 (17A2) and 0.5 μg/ml anti-CD28 (3751) LEAF antibodies (both purchased from BioLegend) in PBS, and were supplemented with 1,000 IU/mL recombinant murine IL-2 (PeproTech, Rocky Hill, NJ).

Flow cytometry—Purified T cells were analyzed using flow cytometry (CytoFLEX, Beckman Coulter, Lakeview Indianapolis, IA) and sorted by FACS (BD FACSAria™ III, BD Biosciences, Franklin Lakes, NJ). Datasets were analyzed using FlowJo software (Tree Star). mAbs for anti-TRP1 conjugated to FITC or specific for the following mice antigen were used: (Alexa Fluor 647 or Brilliant Violet 421) CD3 (clone 17A2), (Phycoerythrin) CD4 (clone RM 4-4), (Brilliant Violet 605) CD8 (clone 53-6.7), (Alexa Fluor 488) CD11b (clone M1/70), (APC/Cy7) CD44 (IM7), (Phycoerythrin/Cy7) CD62L (MEL-14), (Alexa Fluor 647) FcRIV (clone 9E9), (Brilliant Violet 421) TCRb (H57-597), (Allophycocyanin) MHCII(M5/114.15.2), (Fluorescein) FcRI (clone X54-5/7.1), (Phycoerythrin/Cy7) FcRII/III (93). For human specific Ags were used: (Alexa Fluor 488) CD3 (HIT3a), (Alexa Fluor 594) CD4 (RPA-T4), (Allophycocyanin) CD19 (HIB19), (Alexa Fluor 647) CD8 (HIT8a), (Brilliant Violet 650) CD11c (3.9), (Alexa Fluor 647) CD16 (3G8), (PerCp/Cy5.5) CD32 (FUN-2), (Brilliant Violet 421) CD64 (10.1), (Allophycocyanin/Cy7) CD45RO (UCHL1), (Phycoerythrin/Cy7) CD45RA (HI100). All Abs were purchased from BioLegend. Cells were suspended in FACS buffer consisting of HBSS with 2% FCS and 0.05 mM EDTA.

PCR amplification of CD3 and FcγRI—Total RNA was purified from CD11b⁺, FcγRI⁺ and FcγRI^{neg} CD4⁺ sorted cells using RNeasy Micro Kit (Qiagen, Valencia, CA), and was quantified using NanoDrop One (Thermo Fisher Scientific, Pittsburgh, PA). Reverse-transcription was performed using qScript cDNA Synthesis Kit (Quanta biosciences, Beverly, MA) according to manufacturer's protocol. cDNA samples were analyzed by PCR for the detection of FcγRI sequence using AGACACCGCTACACATCTGC (SEQ ID NO: 1) and GGGAAGTTTGTGCCCCAGTA (SEQ ID NO: 2) primers, and CD3 epsilon polypeptide sequence using GCATTCTGAGAGGATGCGGT (SEQ ID NO: 3) and TGGCCTTGGCCTTCCTATTC (SEQ ID NO: 4) primers, and were analyzed by agarose gel electrophoresis.

In vivo tumor models—For melanoma tumor studies, 2×10⁵ B16F10 cells suspended in 50 μL DMEM were injected s.c. to C57BL/6 mice above the right flank and the size of growing tumors was measured twice a week using calipers. When tumors reached 120 mm², mice were sacrificed for ethical considerations. Treatment was applied at day 8 and day 12 post injection, or when tumors reached 20 mm² (day 0 and day 4). For a triple negative breast cancer model, 2×10⁵ 4T1 cells in 30 μL DMEM were injected into fat pad number five of a 12 week old female Balb/c mouse. At day 12, mice were sacrificed and CD4⁺ T cells from DLN, tumors, and non-DLN were analyzed.

Tumor immunotherapy—Animals were injected intratumorally with 80 μg anti-CD40 (clone FGK4.5; BioXCell) and 10 μg TNFα (BioLegend) and with or without 100 μg/mouse anti-human/mouse TRP1 IgG antibodies (clone TA99; BioXCell). 100 μg/mouse of anti-Chicken Ovalbumin (clone TOSGAA1; BioLegend) were used as control.

Adoptive T Cell Transfer—C57Bl/6 mice were injected s.c. with 2×10⁵ B16F10 tumor cells. On days 12 and 14, mice were injected intratumorally with 80 μg anti-CD40 (clone FGK4.5; BioXCell), 1 μg IFNγ (Biolegend) and/or 200 μg anti-TRP1. On day seven, mice were euthanized and the tumors and draining lymph nodes were removed and dissociated to obtain single cell suspensions. Following, T cells were enriched using magnetic beads (EasySep, StemCell technologies) and further sorted by FACSAriaII as FCS^{lo}/SSC^{lo}/TCRβ⁺/MHCII^{neg} cells. T cells were cultured in T cell medium containing 1,000 IU/mL IL-2 (Peprotech) on culture plates coated with 0.5 μg/mL of anti-CD3. Following 9-12 days, T cells were gently collected and a total of 1×10⁶ cells were injected intravenously into mice bearing tumors with an average size of 30-50 mm².

Immunohistochemistry—For frozen sections, tissues were fixed in 4% paraformaldehyde for 1 hour and equilibrated in a 20% sucrose solution overnight. Following, tissues were embedded in frozen tissue matrix (Scigen O.C.T. Compound Cryostat Embedding Medium, Thermo Fisher Scientific), and frozen at −80° C. The 5-μm-thick sections were blocked with 5% BSA and stained with 1:100 diluted primary antibodies. Staining was performed using anti-CD3 (clone 17A2), anti-CD4 (RM4-4), anti-TCRβ (H57-597), anti-FcRI (X54-5/7.1), anti-FcRII/III (93), anti-FcRIV (9E9). Nuclei were counterstained with Hoechst 33342 (Fluka). Microscopy was performed with a ZEISS LSM 800 confocal microscope and analyzed using ZEN software (ZEISS, Germany).

Confocal microscopy—B16-Wassabi and CD4⁺ T cells were co-cultured on glass-bottom confocal plates (Cellvis, Mountain View, CA) in T cell medium without IL-2 and incubated overnight under standard conditions. Cells were further incubated for 1 h with BV421-conjugated anti-CD107 (BioLegend) at 1:100 dilution. Images were collected using a Zeiss LSM800 confocal laser scanning microscope and analyzed using ZEN software (Carl Zeiss Microscopy).

Preparation of Fab2′ fragments—Anti-TRP1 Ab (clone TA99; BioXCell) was dialyzed against 20 mM sodium acetate pH 4.5 and digested with agarose-pepsin beads (Goldbio, St. Louis, MO) for 16 hours in 37° C. incubator with rotation. Next, the sample was centrifuged and supernatant was collected, dialyzed against PBS pH 7.4 and incubated with protein-A agarose beads (Santa Cruz Biotechnology, Dallas, TX) for 2 hours with rotation. Fab2′ fraction was collected after centrifugation and was analyzed by PAGE.

Killing assay—CD4⁺ T cells were co-cultured with B16 target cells (30,000 cells per well) at a ratio of 1:2 (T:E) in a round bottom 96-wells plate with or without the following antibodies: anti-Chicken Ovalbumin (clone TOSGAA1; BioLegend), anti-TRP-1 (clone TA99; BioXCell), or anti-TRP-1 Fab2′. Following 24 hours and 48 hours of incubation medium was replaced with PBS, and fluorescence intensity of wasabi (excitation 485 nm emission 528 nm) was measured by Synergy H1M plate reader (BioTek, Winooski, VT). Following 48 hours, cells were stained with Annexin V (Biolegend) for 15 minutes and propidium iodide for 2 minutes on ice and staining levels were analyzed by flow cytometry.

Statistical analyses—Each experiment was performed three times. Each experimental group consisted of at least three mice. Significance of results was determined using the nonparametric one-way ANOVA, when multiple groups are analyzed, or nonparametric Student's t-test.

### Results

Adoptive transfer of CD4⁺ T cells and tumor-binding antibodies induce direct tumor lysis—In a previous study, the changes that occur following effective immunotherapy in a mouse model of spontaneous melanoma, as well as in melanoma patients treated with GM-CSF and CTLA-4 were analyzed. This analysis revealed that effective immunotherapy is highly associated with massive expansion of a number of antigen-experienced CD4⁺ T-cell populations in various anatomical organs¹⁹. In a follow-up study, the present inventors characterized which organ contains the most potent tumor-reactive CD4⁺ T cells. To this end, effector CD4⁺ T cells were isolated from the blood, draining lymph node (DLN) and B16 melanoma tumors of wild type (WT) C57BL/6 mice, and transferred by i.v. injection to WT C57BL/6 mice bearing B16 melanoma cells, in combination with antibodies against the melanoma antigen TRP1 (gp75, FIG. 1A). While injection of effector CD4⁺ T cells from blood had only a minor effect on tumor regression, injection of CD4⁺ T cells from the tumor and DLN induced a significant, long-lasting tumor regression (FIGS. 1B-C). In the next step, the present inventors assessed whether transferred CD4⁺ T cells directly kill tumor cells, or rather mediate their killing by activating other effector T cells. Thus, RAG-deficient mice (RAG^{-/-}) were challenged with B16 cells, and tumors were allowed to grow for ten days. Following, RAG^{-/-} mice were injected with 1×10⁶ effector CD4⁺ derived from WT C57BL/6 tumor-bearing mice with or without anti-TRP1 antibodies. Interestingly, the efficacy of this treatment in RAG^{-/-} mice was comparable to that of immune-competent mice, suggesting that tumor lysis is induced directly by the transferred antibodies and CD4⁺ T cells (FIG. 1D). Next, whether the specificity of the antibodies and T-cell receptors (TCRs) played a role in inducing tumor regression was evaluated. To this end, CD4⁺ T cells bearing a single TCR were isolated from OT-II, which recognize the irrelevant Ovalbumin (Ova) epitope, or from RAG1⁻B^{W} TRP-1 TCR mice, which recognize a peptide derived from TRP1. Following, the effector T cells were injected to WT C57BL/6 mice bearing B16 melanoma cells in combination with an antibody against Ova, which is not expressed on B16, or with an antibody against the tumor antigen TRP1. Adoptive transfer of effector T cells alone were almost inert and tumor growth in these groups was comparable to that of untreated mice. Similarly, injection of Ova-reactive T cells with antibodies had only marginal effect on tumor growth. In sharp contrast, injection of TRP1-reactive CD4⁺ T cells along with anti-TRP1 antibodies, but not anti-Ova antibodies, induced a complete and durable tumor eradication (FIG. 1E).

Taken together, these results demonstrate that cytotoxic activity of CD4⁺ requires that both the TCR and the antibodies target the tumor cells.

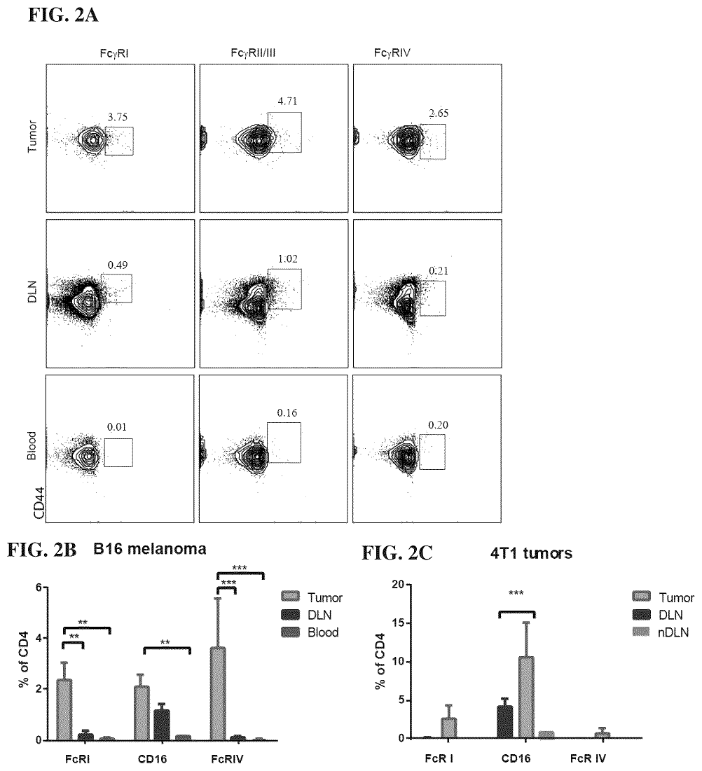

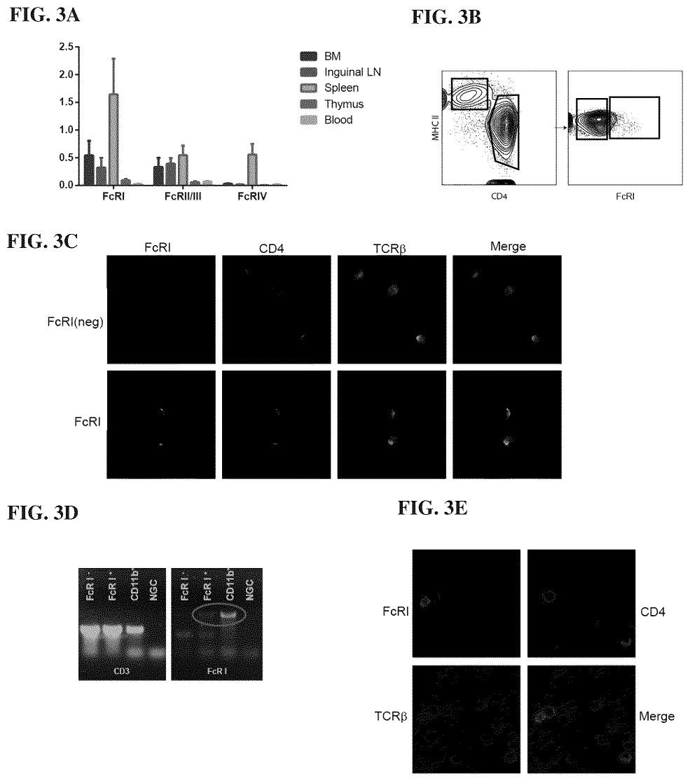

A subset of CD4⁺ T cells in lymphoid and cancerous organs express Fcγ receptors—Although it is widely recognized that T cells do not express Fcγ receptors (FcγR), in light of the results described hereinabove, the present inventors decided to revisit this notion. To this end, tumors, DLN, and PB were harvested from WT C57BL/6 mice bearing B16 melanoma cells, and the expression patterns of FcγR on CD4⁺ T cells was analyzed by FACS. This analysis revealed that about five percent of the tumor-infiltrating CD4⁺ T cells express all three types of FcγR [FcγRI (CD64, also referred to herein as FcγRIα), FcγRII/III and FcγRIV] in levels comparable to that of antigen presenting cells, which are known to express these receptors (FIG. 2A). Lower (yet detectable) percentages of CD4⁺ T cells expressing FcγR were also observed in the DLN, but not in peripheral blood (FIG. 2B). Following, whether this subset exists in another tumor model, or rather is limited to B16 melanoma was evaluated. Indeed, all three FcγR were expressed on CD4⁺ T cells in the tumors and DLN of Balb/c mice bearing 4T1 breast carcinoma cells, yet with different expression patterns (FIG. 2C). The inventors also tested whether this population exists in naïve mice, or rather is induced exclusively during tumor progression. To this end, various organs were harvested from naïve mice, and FcγR expression on T cells was analyzed. T cells expressing FcγR were found in lymph nodes, spleen, and bone marrow (BM), but not in the blood or thymus (FIG. 3A). These T cells were completely absent in RAG^{-/-} mice, suggesting that their maturation is dependent on TCR rearrangement (data not shown). To ensure that these are indeed T cells, splenic cells were applied on a Ficoll gradient, enriched on CD4-magnetic beads, and FcγRI+ and FcγRI^{neg}/CD3+/MHCII^{neg/dull} cells were sorted (FIG. 3B). Confocal analysis indicated that both subsets share similar morphology and size and have identical cell membrane TCRβ staining. Additional staining further indicated that FcγRI is expressed on the cell membrane in close proximity to CD4 molecules (FIG. 3C). These results were further validated by amplifying FcγRI transcripts by PCR. Consistent with the FACS and confocal results, it was found that FcγRI gene transcript is expressed in FcγRI⁺/CD3⁺/MHCII^{neg/dull} CD4⁺ T cells, but not conventional FcγRI negative CD4⁺ T cells (FIG. 3D). Moreover, histological sections staining of naïve spleen and tumors further indicated that these cells are exclusively located at the margins of the T cell zone (FIG. 3E).

Tumor specific CD4⁺ T cells expressing FcγRI induce effective tumor cell lysis—In the next step, whether the expression of FcγR on T cells is functional, or merely a surface marker was tested. To this end, splenic CD4⁺ T cells that either express or do not express FcγRI were isolated from wild type (WT) C57BL/6 control mice and incubated overnight with B16 tumor cells. Incubation of FcγRI⁺/CD4⁺ T cells, but not FcγRI^{neg}/CD4⁺ T cells, with B16 in combination with anti-TRP1 antibodies induced a remarkable tumor cell lysis. Tumor cell lysis was completely abrogated when FcγRI+/CD4⁺ T cells were incubated with anti-ovalbumin antibodies, or anti-TRP1 Fab2′. In addition, incubation of FcγRI+/CD4⁺ isolated from OT-II mice with B16 and anti-TRP1 did not induce tumor killing, suggesting that the TCR must target tumor antigens (FIGS. 4A-B).

### Example 2

### Exogenous Expression of FcγRI and FcRγ in CD4+ and CD8+ T Cells Induces Effective Tumor Cell Lysis

### Materials and Methods

Mice and cell lines—As described in Example 1 hereinabove. In addition, tdTomato B16F10 cells were obtained by infecting B16F10 cells by lentivirus containing pLVX-H2B-tdTomato, followed by sorting by FACS (BD FACSAria™ III, BD Biosciences, Franklin Lakes, NJ) for the high-expressing tdTomato population.

T cell isolation—Spleens were removed from WT C57BL/6 mice and mashed through 70 μM cell strainer. Following, splenocytes were collected and incubated with anti-CD4, or anti-CD8 magnetic beads (MojoSort™ Nanobeads, BioLegend, Carlsbad, CA) according to manufacturer's instructions.

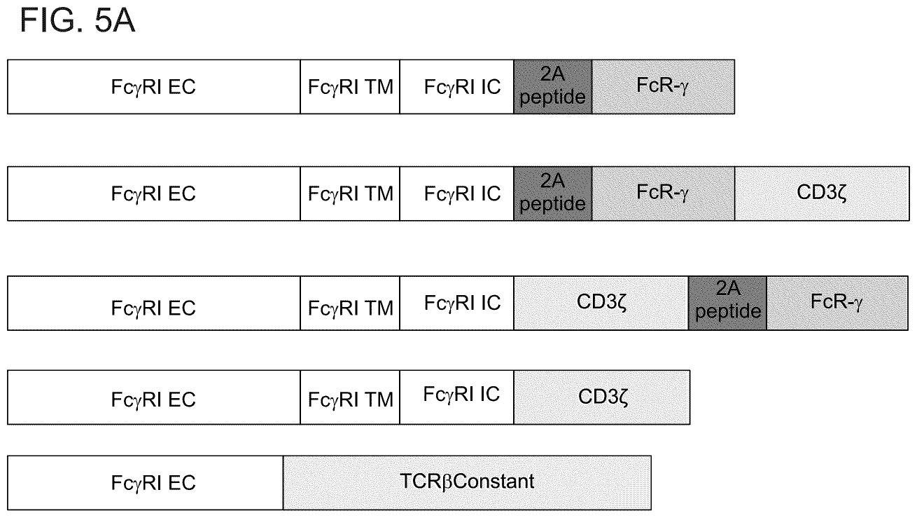

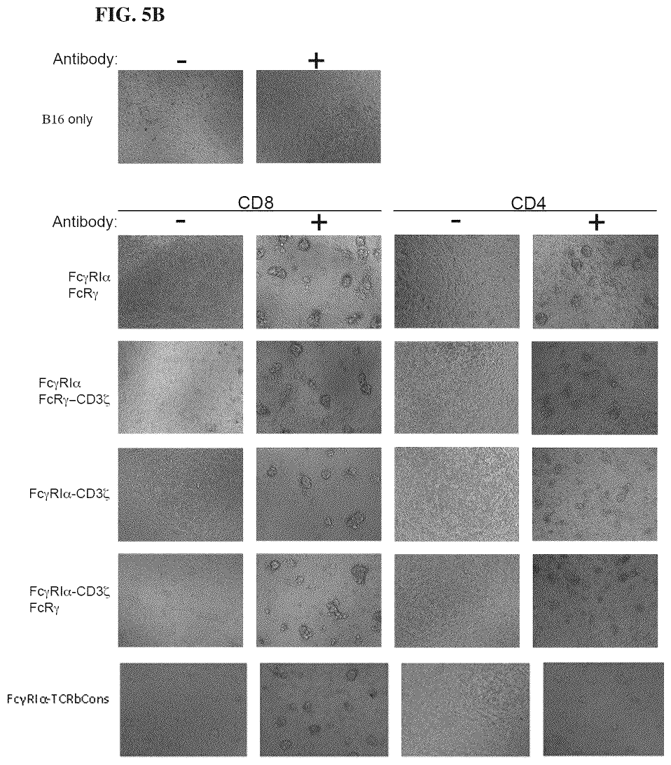

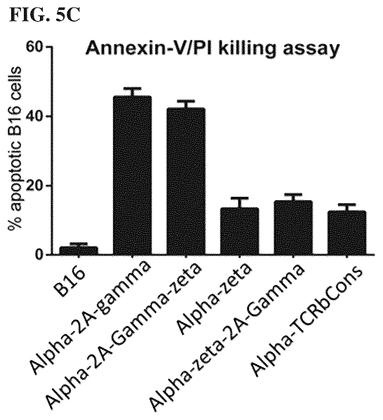

T cell transduction—Three retrovirus packed plasmids were generated: TRP1-reactive TCR (SEQ ID NOs: 35-36), FcγRI (SEQ ID NOs: 5-6), and Fc receptor signaling gamma chain (FcRγ, SEQ ID NOs: 15-16). In addition, several constructs were generated to express FcγRI, FcRγ and/or TCR CD3zeta chain; FcγRI extracellular domain and TCRβ constant region; and FcγRI extracellular domain, CD8 hinge+transmembrane domains and FcRγ in single plasmids (see FIGS. 5A, 6A, 7 and 11A), SEQ ID Nos: 21-28 and 41-44). Specifically, inserts of the fusion sequences were synthesized by GeneART (Thermo Fisher Scientific) into pMK vectors and were further cloned into pMIGII using EcoRI/XhoI sites upstream to IRES-GFP sequences. Clones were verified by pBABE5′ and IRES-Rev primers sequencing (HyLabs Israel). Histone H2B sequence was amplified with AATAACACTAGTGCCACCATGCCTGAACCGGCAAAAT (SEQ ID NO: 45) and AACAACCCCGGGACTTGTCGTCATCGTCTTTGT (SEQ ID NO: 46) primers and cloned into pLVX vector (Clontech) containing EF1 promoter into SpeI/XmaI sites in frame with tdTomato. Sequence was verified by MSCv forward and tdTomato reverse primers (HyLabs Israel).

Retroviral infection: Mouse CD4⁺ and CD8⁺ T cells were isolated from mouse blood and infected with the above constructs as follows: Platinum E cells were plated on 10 cm culture plates and co-transfected with 2:1 molar ratio of pMIGII⁴⁵ and PCL-Eco plasmids using Polyplus jetPRIME® reagent (Polyplus transfections). Following 24 hours, media was replaced with complete DMEM supplemented with 0.075% Sodium Bicarbonate. Media-containing viruses were collected after 24 hours and 48 hours and centrifuged for 1 hour at 100,000 g. Pellet was resuspended gently in 1 mL media and let to recover overnight at 4° C. Prior to infection, splenic CD4⁺ T cells or splenic CD8⁺ T cells were incubated on plate pre-coated with anti-CD3 (0.5 μg/mL) in T cell media containing high-dose IL-2 (1,000 IU/ml). Next, 0.3 mL of concentrated retroviruses were added to every 2×10⁶ CD4+ or CD8⁺ T cells with 10 μg/mL polybrene. Cells were incubated for 30 minutes in 37° C., 5% CO₂ and centrifuged at 37° C. 1,200 rpm for 1 hour. Following, 80% of medium was replaced and T cells were cultured for additional three days in T cell media containing high-dose IL-2.

Lentiviral infection: HEK-293FT cells were transfected with pLVX plasmids containing H2B-tdTomato under EF1 promoter together with psPAX2 (Addgene plasmid #12260) and pCMV-VSV-G (Addgene plasmid #8454). Media-containing viruses were collected following 24 and 48 hours. For infection, B16F10 cells were incubated with viruses and 100 μg/mL polybrene (Sigma Aldrich, Merck, Israel) for 30 minutes followed by 30 minutes centrifugation before medium was replaced. Following three days, cells that expressed tdTomato were sorted by FACSAriaII.

In vivo tumor models—As described in Example 1 hereinabove.

Adoptive T Cell Transfer—C57Bl/6 mice were injected s.c. with 2×10⁵ B16F10 tumor cells. Following 9-12 days, a total of 1×10⁶ transduced T cells were injected intravenously into mice bearing tumors with an average size of 30-50 mm² with or without 200 μg anti-TRP1.

Killing assay—CD4+ or CD8⁺ T cells were co-cultured with B16 target cells (30,000 cells per well) at a ratio of 1:2 (T:E) in a round bottom 96-wells plate with or without anti-TRP-1 (clone TA99; BioXCell). Following 48 hours of incubation images where taken under ×100 magnitude in inverted light microscope. In addition, following 48 hours, cells were stained with Annexin V (Biolegend) for 15 minutes and propidium iodide for 2 minutes on ice and staining levels were analyzed by flow cytometry. IncuCyte imager killing assay were conducted by culturing 10⁴ H2B-tdTomato B16F10 target cells in 96 wells plate. Two hours later 2×10⁴ T cells were added with or without 15 μg anti-TRP-1 antibodies in 200 μl medium and were imaged by incuCyte S3 imager (Sartorius) for at least 24 hours. Images were then used to calculate numbers of target cells by incuCyte software.

Confocal microscopy—CD4⁺ and CD8⁺ T cells were plated on glass-bottom confocal plates and stained using anti-CD3 (clone 17A2), anti-TCRβ (H57-597), anti-FcRI (X54-5/7.1). Images were collected using a Zeiss LSM800 confocal laser scanning microscope and analyzed using ZEN software (Carl Zeiss Microscopy).

Statistical analyses—As described in Example 1 hereinabove.

### Results

In the next step the inventors tested whether the killing mechanism described in Example 1 hereinabove can be mimicked in FcγRI^{neg}/CD4⁺ T cells. To this end, splenic CD4⁺ T cells were infected with three retrovirus packed plasmids: TRP1-reactive TCR, FcγRI and/or Fc receptor signaling gamma chain (FcRγ), and plated with B16 tumor cells (FIG. 4C). Importantly, CD4⁺ T cells infected with tumor-specific TCR, FcγRI and the gamma chain induced the most substantial killing response, as can be seen by CD107a on the T cell membrane and cell death of tumor cells coated with antibodies. Representative microscope images of antibody mediated B16 killing by TCR-FcγRI-TcRγ infected CD4⁺ are shown on the right panel (FIG. 4C). Following the infected CD4⁺ T cells were tested in an adoptive transfer model, with or without an anti-TRP1 antibody, to evaluate the killing activity of the cells in vivo. As shown in FIG. 4D, same as in-vitro, in the in-vivo model, expression of TCR of TRP1 together with FcγRI and the signaling gamma chain, in combination with an anti-TRP1 antibody, mediated tumor eradication.

Subsequently, the following constructs were cloned (FIGS. 5A, 6A and 7): FcγRIα and FcRγ separated by T2A sequence (SEQ ID Nos: 21-22), FcγRIα T2A FcRγ-CD3ζ (zeta chain ITAMS) fusion (SEQ ID Nos: 23-24), FcγRIα-CD3ζ fusion (SEQ ID Nos: 25-26), FcγRIα-CD3ζ T2A FcRγ (SEQ ID NO: 27-28) and FcγRIα-TCRβ constant region (SEQ ID NO: 41-42). These plasmids were packed into retrovirus, and used to infect CD4⁺ and CD8⁺ T cells. The transduced T cells were further co-cultured and tested for B16 killing activity with an anti-TRP1 antibody (FIG. 5B). To compare the levels of killing mediated by the different setting of receptors, the B16 cells that were co-cultured with the transduced CD8⁺ T cells were stained with annexin-V/PI and analyzed by flow cytometry (FIG. 5C).

Taken together, these results demonstrate that concomitant signaling through FcγRI and the FcRγ signaling chain can exert killing capacities in conventional CD4⁺ and CD8⁺ T cells whenever the target cells are coated with antibodies. In addition, a comparison shows the advantage of separation of the FcγR signaling molecule is more potent than fusion of ITAMS of CD3ζ signaling or TCRbeta to the FcγRI receptor.

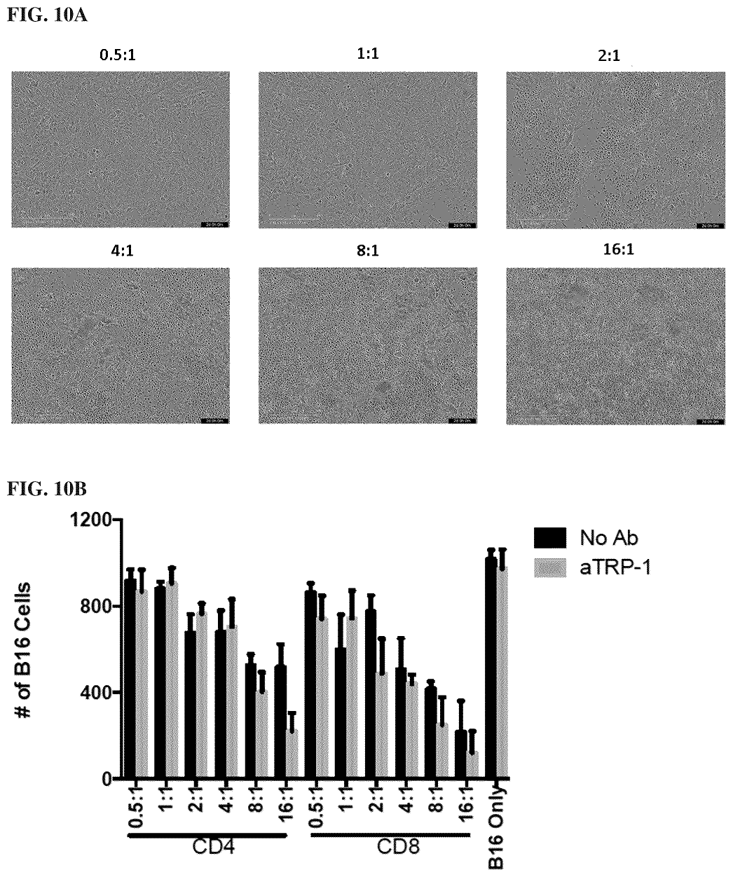

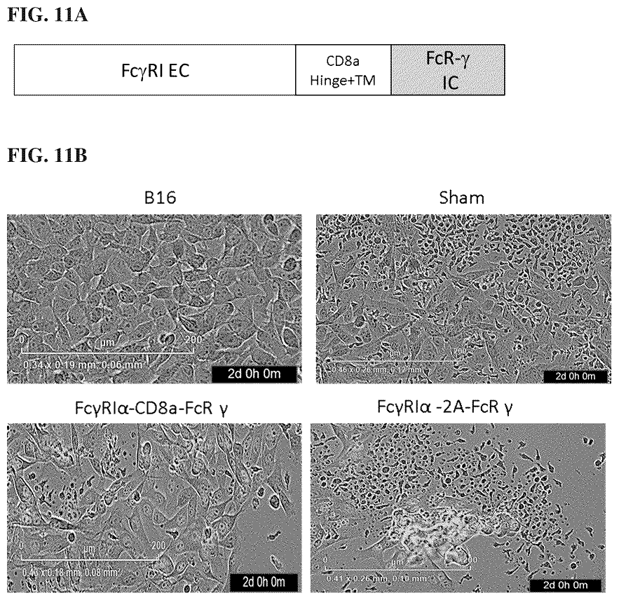

To validate membrane localization of the FcγRIα-2A-FcRγ construct, cells were stained for TCRβ and CD3, and for FcγRIα. Confocal analysis indicated that FcγRIα was uniformly localized on T cell membrane (FIG. 8). Following, the killing ability of T cells infected with the FcγRIα-2A-FcRγ construct was evaluated using B16 cells which express histone H2B-tdTomato. Initially, B16-H2B-tdTomato were cultured in serial concentrations ranging from 24 cells to 50,000 per well, imaged in incuCyte and counted by incuCyte analysis tool which detect and count the red fluorescent nuclei in a field captured by the camera. The graph in FIG. 9 shows a direct correlation between the amount of cell cultured and numbers of cell counted in a field. Consequently, the incuCyte imaging system was used to evaluate killing of B16-H2B-tdTomato by anti-TRP-1 antibody and T cells expressing FcγRIα-2A-FcRγ cultured in different effector:target ratios, ranging from 0.5:1 to 16:1. Representative images (FIG. 10A) and target cells numbers (FIG. 10B) after 48 hours show that both CD8⁺ and CD4⁺ T cells killed the tumor cells when the effector:target ratio is 8 to 1, or higher.

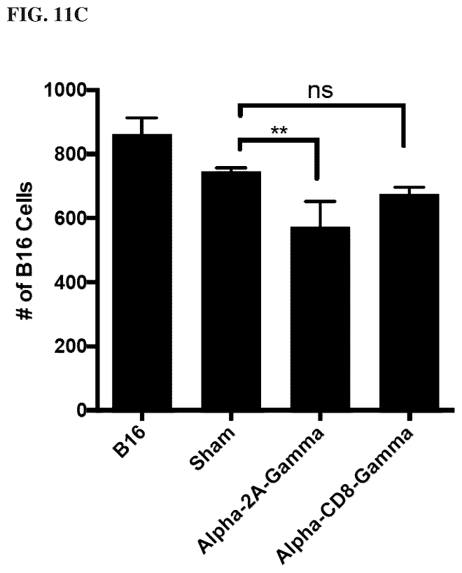

Subsequently, an additional construct was cloned (FIG. 11A): FcγRIα extracellular domain-CD8 hinge and transmembrane domain-FcRγ (SEQ ID NO: 43-44). The construct was expressed in CD8+ T cells and their killing ability was evaluated using B16 cells expressing the histone H2B-tdTomato (FIGS. 11B-C). The results show the advantage of expressing two distinct polypeptides, one comprising the ligand binding domain of FcγRIα and the other comprising FcRγ, as compared to a single polypeptide expressing both.

### Example 3

Mouse and Human CD4+ and CD8+ T Cells Exogenously Expressing FcγRI and FcRγ have Anti-Tumor Effects

### Materials and Methods

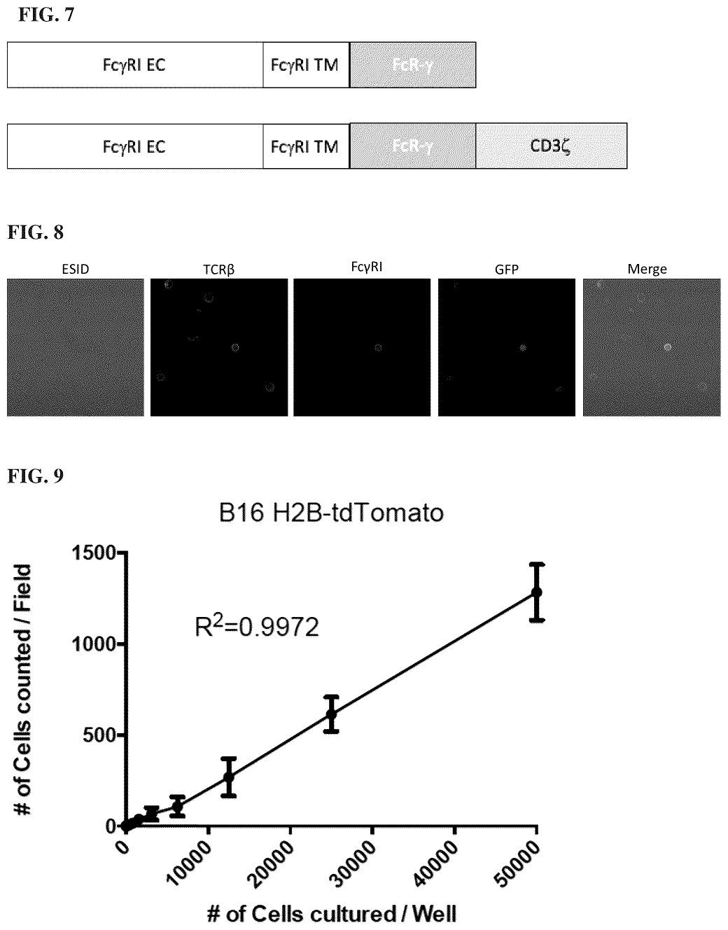

T cell transduction—Several constructs are generated as described in Example 2 hereinabove. In addition, additional constructs for expressing FcγRI, FcRγ and TCR CD3zeta chain as a single polypeptide are generated (see FIG. 7, SEQ ID Nos: 29-32). Mouse CD4+ and CD8⁺ T cells are isolated from mouse blood and infected with the above constructs as described in Example 2 hereinabove. Human CD4⁺ and CD8⁺ T cells are isolated from the blood of healthy donors or from the blood of melanoma patients refractory to treatment with Atezolizumab and infected with the above constructs.

Measuring in vitro the cytotoxic activity of the transduced mouse T cells—Transduced T cells are co-cultured with B16, 4T1, or MC38 tumor cells, which express high levels of PDL1, with or without anti-PDL1 antibodies (BioXCell). At several time points, tumor cell lysis is measured using a fluorescence live cells assay created by a Biotek HIM plate reader.

Measuring in vitro the cytotoxic activity of the transduced patient-derived T cells—Transduced T cells isolated from healthy donors are incubated with SK-Mel-5 and A375 tumor cell lines that express PDL1, with or without Atezolizumab. At several time points, tumor cell lysis is measured. Furthermore, transduced T cells isolated from the blood of melanoma patients' refractory to treatment with Atezolizumab are co-cultured with autologous melanoma tumor cells, with or without Atezolizumab. At several time points, tumor cell lysis is measured by a fluorescence live cells assay using a Biotek HIM plate reader.

Testing in vitro the specificity of the transduced T cell—Since T cells can also express PDL1, though usually at a low level, the concentration of antigens that elicit killing of target cells is tested. To this end, macrophages, B cells, and endothelial cells are isolated from naive mice and healthy human donors and activated with IFNγ, to induce PDL1 expression. Following the cells are incubated overnight with the transduced T cells, with or without anti-PDL1 antibodies, and cell mortality is determined by annexin V and propidium iodide (PI) staining.

Testing the capacity of the transduced mouse T cells to eradicate established solid tumors—Mice are injected with B16 cells, MC38, or 4T1, all of which express high levels of PDL1, but are refractory to blocking antibodies. Once tumors are established, mice are treated by i.v. injection with the transduced mouse T cells, with or without mouse anti-mouse PDL1 antibodies and tumor burden is monitored. In addition, tumors are analyzed by flow cytometry for their T cell infiltration, expansion and IFNγ secretion. Tumor cell apoptosis is determined by tunnel staining under confocal microscopy.

Testing the capacity of the transduced human T cells to eradicate human tumors—Tumor cell lines SK-Mel-5 and A375 that express PDL1, are transplanted into nude-scid-IL2Rγ^{-/-} mice (NSG), and left to grow to a palpable size. Following, mice are injected with transduced human T cells with or without Atezolizumab and tumor growth is monitored. In addition, tumors are analyzed by flow cytometry for their T cell infiltration, expansion and IFNγ secretion. Tumor cell apoptosis is determined by tunnel staining under confocal microscopy.

Testing the capacity of the transduced tumor T cells to kill refractory human tumors—Fresh tumor samples and PBMC are obtained from patients, which their tumors express high levels of PDL1 and are undergoing respective surgery. Transduced cell lines are established from cancer patients and injected into NSG mice. Once tumors reach a palpable size, mice are injected with transduced autologous T cells with or without Atezolizumab and tumor growth is monitored.

Assessing mouse health, signs of cytokine storm and tumor-lysis syndrome—Alongside the monitored tumor growth in syngeneic and NSG mice, mice are examined routinely for their wellbeing following treatments. To this end, mice are weighed every other day and assessed for their level of activity, as well as signs of dermatitis, diarrhea, and acute pain. In addition, mice are bled twice a week from the retinal tear and tested for serum levels of CRP, MCP-1, IL-6, TNFα, IFNγ and IL-1. Serum samples are also tested for metabolic abnormalities, including levels of potassium, phosphate, calcium, uric acid, glucose, creatinine, and albumin, and the liver enzymes ALT, AST, ALP.

Testing off-target tumor cytotoxicity and signs of autoimmunity—Once the experiments are terminated, mice treated with the transduced T cells with or without Atezolizumab, are analyzed by staining serial histological sections. Excessive lymphocyte proliferation in lymphoid organs is tested by Ki67 staining and liver, adrenal cortex, salivary glands, kidney, heart, skin, and colon are tested for immune infiltrates by staining for T cells, B cells, and myeloid cells.

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims.

It is the intent of the applicant(s) that all publications, patents and patent applications referred to in this specification are to be incorporated in their entirety by reference into the specification, as if each individual publication, patent or patent application was specifically and individually noted when referenced that it is to be incorporated herein by reference. In addition, citation or identification of any reference in this application shall not be construed as an admission that such reference is available as prior art to the present invention. To the extent that section headings are used, they should not be construed as necessarily limiting. In addition, any priority document(s) of this application is/are hereby incorporated herein by reference in its/their entirety.

### REFERENCES

### (Other References are Cited Throughout the Application)

ADDIN EN.REFLIST 1 Restifo, N. P., Dudley, M. E. & Rosenberg, S. A. Adoptive immunotherapy for cancer: harnessing the T cell response. Nat Rev Immunol 12, 269-281, doi:10.1038/nri3191 nri3191 [pii](2012).
2 Rosenberg, S. A. Decade in review-cancer immunotherapy: entering the mainstream of cancer treatment. Nat Rev Clin Oncol 11, 630-632, doi:10.1038/nrclinonc.2014.174 nrclinonc.2014.174 [pii] (2014).
3 Wu, R. et al. Adoptive T-cell therapy using autologous tumor-infiltrating lymphocytes for metastatic melanoma: current status and future outlook. Cancer J 18, 160-175, doi:10.1097/PPO.0b013e31824d4465 (2012).
4 Gross, G., Gorochov, G., Waks, T. & Eshhar, Z. Generation of effector T cells expressing chimeric T cell receptor with antibody type-specificity. Transplant Proc 21, 127-130 (1989).
5 Kalos, M. et al. T cells with chimeric antigen receptors have potent antitumor effects and can establish memory in patients with advanced leukemia. Sci Transl Med 3, 95ra73, doi:10.1126/scitranslmed.3002842 3/95/95ra73 [pii] (2011).
6 Pardoll, D. M. The blockade of immune checkpoints in cancer immunotherapy. Nat Rev Cancer 12, 252-264, doi:10.1038/nrc3239 nrc3239 [pii] (2012).
7 Hodi, F. S. et al. Improved survival with ipilimumab in patients with metastatic melanoma. The New England journal of medicine 363, 711-723, doi:10.1056/NEJMoa1003466 (2010).
8 Gross, G. & Eshhar, Z. Therapeutic Potential of T Cell Chimeric Antigen Receptors (CARs) in Cancer Treatment: Counteracting Off-Tumor Toxicities for Safe CAR T Cell Therapy. Annu Rev Pharmacol Toxicol 56, 59-83, doi:10.1146/annurev-pharmtox-010814-124844 (2016).
9 Sharma, P. & Allison, J. P. The future of immune checkpoint therapy. Science 348, 56-61, doi:10.1126/science.aaa8172 348/6230/56 [pii] (2015).
10 Gajewski, T. F., Schreiber, H. & Fu, Y. X. Innate and adaptive immune cells in the tumor microenvironment. NatImmunol 14, 1014-1022, doi:10.1038/ni.2703 ni.2703 [pii] (2013).
11 Dunn, G. P., Bruce, A. T., Ikeda, H., Old, L. J. & Schreiber, R. D. Cancer immunoediting: from immunosurveillance to tumor escape. Nat Immunol 3, 991-998, doi:10.1038/ni1102-991 (2002).
12 Khong, H. T. & Restifo, N. P. Natural selection of tumor variants in the generation of “tumor escape” phenotypes. Nat Immunol 3, 999-1005, doi:10.1038/ni1102-999 ni1102-999 [pii] (2002).
13 Gabrilovich, D. I. & Nagaraj, S. Myeloid-derived suppressor cells as regulators of the immune system. Nat Rev Immunol 9, 162-174, doi:10.1038/nri2506 nri2506 [pii] (2009).
14 Whiteside, T. L. The tumor microenvironment and its role in promoting tumor growth. Oncogene 27, 5904-5912, doi:10.1038/onc.2008.271 onc2008271 [pii] (2008).
15 Mantovani, A. & Sica, A. Macrophages, innate immunity and cancer: balance, tolerance, and diversity. Curr Opin Immunol 22, 231-237, doi:10.1016/j.coi.2010.01.009 (2010).
16 Coussens, L. M., Zitvogel, L. & Palucka, A. K. Neutralizing tumor-promoting chronic inflammation: a magic bullet? Science 339, 286-291, doi:10.1126/science.1232227 339/6117/286 [pii] (2013).
17 Rudensky, A. Y., Gavin, M. & Zheng, Y. FOXP3 and NFAT: partners in tolerance. Cell 126, 253-256, doi:S0092-8674(06)00895-6 [pii] 10.1016/j.cell.2006.07.005 (2006).
18 Joyce, J. A. & Fearon, D. T. T cell exclusion, immune privilege, and the tumor microenvironment. Science 348, 74-80, doi:10.1126/science.aaa6204 348/6230/74 [pii](2015).
19 Spitzer, M. H. et al. Systemic Immunity Is Required for Effective Cancer Immunotherapy. Cell 168, 487-502 e415, doi:S0092-8674(16)31738-X [pii]10.1016/j.cell.2016.12.022 (2017).

What is claimed is:

1. A T cell genetically engineered to express two distinct polypeptide, a first polypeptide comprising an amino acid sequence of an Fc receptor common γ chain (FcRγ), said amino acid sequence is capable of transmitting an activating signal; and a second polypeptide comprising an extracellular ligand-binding domain of an Fcγ receptor capable of binding an Fc ligand and an amino acid sequence capable of recruiting said first polypeptide such that upon binding of said Fc ligand to said extracellular ligand-binding domain of said Fcγ receptor said activating signal is transmitted, wherein said T cell is suitable for adoptive therapy in a subject in need thereof.

2. The T cell of claim 1, wherein said amino acid sequence capable of recruiting said first polypeptide comprises the transmembrane domain and/or the cytoplasmic domain of an Fc receptor.

3. The T cell of claim 2, wherein said Fc receptor is Fcγ receptor.

4. The T cell of claim 1, wherein said Fcγ receptor is CD64.

5. The T cell of claim 1, wherein said first polypeptide is less than 25 kDa in molecular weight.

6. The T cell of claim 1, wherein said first polypeptide does not comprise a target-binding moiety.

7. The T cell of claim 1, wherein said first polypeptide does not comprise a scFv; and/or wherein said second polypeptide does not comprise a scFv.

8. The T cell of claim 1, wherein said T cell does not express a chimeric antigen receptor (CAR).

9. A method of treating a disease associated with a pathologic cell in a subject treated with a therapeutic composition comprising an Fc domain, said therapeutic composition being specific for said pathologic cell, the method comprising administering to the subject a therapeutically effective amount of the T cells of claim 1, thereby treating the disease in the subject.

10. A method of treating a disease associated with a pathologic cell in a subject in need thereof, the method comprising administering to the subject a therapeutically effective amount of the T cells of claim 1; and a therapeutic composition comprising an Fc domain, said therapeutic composition being specific for said pathologic cell, thereby treating the disease in the subject.

11. An article of manufacture comprising a packaging material packaging the T cells of claim 1 and a therapeutic composition comprising an Fc domain.

12. The article of manufacture of claim 11, wherein said therapeutic composition is specific for a pathologic cell.

13. The method of claim 9, wherein said therapeutic composition is an Fc-fusion protein.

14. The method of claim 9, wherein said therapeutic composition is an antibody.

15. The method of claim 9, wherein said disease is cancer and wherein said pathologic cell is a cancerous cell.

16. The method of claim 15, wherein said cancer is selected from the group consisting of melanoma, adenocarcinoma, mammary carcinoma, colon cancer, ovarian cancer, lung cancer and B-cell lymphoma.

17. The T cell of claim 1, wherein said T cell is a CD4+ T cell.

18. The T cell of claim 1, wherein said T cell is a CD8+ T cell.

19. The T cell of claim 1, wherein said T cell is a proliferating cell.

20. The method of claim 9, wherein said T cells are autologous to said subject.
